(19) United States
(12) Patent Application Publication
(10) Pub. No.: US 2002/0147197 A1
(43) Pub. Date: Oct. 10, 2002

(54) Methods and compositions for enhancing pharmaceutical treatments

(76) Inventors: Michael J. Newman, San Diego, CA (US); William Ross Dixon, La Jolla, CA (US)

Correspondence Address: ONTOGEN CORPORATION, PATENT DEPARTMENT, 6451 EL CAMINO REAL, CARLSBAD, CA, 92009, US

(21) Appl. No.: 10/104,549

(22) Filed: Mar. 20, 2002

Related U.S. Application Data
(63) Continuation-in-part of application No. 09/684,293, filed on Oct. 6, 2000.
(60) Provisional application No. 60/158,322, filed on Oct. 8, 1999.

(51) Int. Cl. A61K 31/5377; A61K 31/454; A61K 31/4178

(52) U.S. Cl. 514/235.8; 514/254.05; 514/326; 514/396; 514/397; 514/400

(57) ABSTRACT

Improved methods are provided for therapeutic and/or preventative treatment to a mammal in which the mammal is protected against the toxicity of active pharmaceutical agents that (i) bind to or are substrates for P-gp, (ii) are taxane analogues, and/or (iii) are inhibitors of tubulin disassembly. Additionally provided are compositions and methods useful for treating cell proliferative disorders. Further provided are methods of increasing the bioavailability of therapeutic and/or preventative treatments in a mammal. Particular embodiments are directed to increasing such bioavailability across the blood-brain barrier.

### CROSS REFERENCE TO RELATED APPLICATIONS

This application claims priority under 35 U.S.C. §119(e)(1) from Provisional Application Serial No. 60/158,322, filed Oct. 8, 1999; and under 35 U.S.C. §120 from co-pending U.S. patent application Ser. No. 09/684,293 filed on Oct. 6, 2000.

### FIELD OF THE INVENTION

The present invention generally relates to improved methods for providing therapeutic and/or preventative treatment to a mammal in which the mammal is protected against the toxicity of an active pharmaceutical agent that (i) binds to or is a substrate for P-glycoprotein, (ii) is a taxane analogue, and/or (iii) is an inhibitor of tubulin disassembly. The present invention further generally relates to compositions and methods useful for treating cell proliferative disorders. The invention further provides methods of increasing the bioavailability of therapeutic and/or preventative treatments in a mammal. Particular embodiments are directed to increasing such bioavailability across the blood-brain barrier.

### BACKGROUND

Multi-drug resistance (“MDR”) is a well-known cellular feature that frequently operates to decrease the efficacy of therapeutic and preventative treatments by pharmaceutical agents. As an integral part of a mammal's natural defense systems against toxic agents, P-glycoprotein (“P-gp”) is expressed to varying degrees throughout the body. P-gp acts at cell membranes as an adenosine triphosphate-dependent efflux pump to actively remove foreign materials from cells, including xenobiotics such as chemotherapy agents. This efflux activity serves an essential protective function, particularly at critical boundaries such as the blood-brain barrier. This activity also occurs, for example, at the lumen of the intestines during absorption of xenobiotics, and in the kidneys. However, in the event that pharmaceutical agents are intended to be introduced into such cells, MDR can substantially lessen or eliminate the intended therapeutic or preventative result of the treatment.

Some cells intrinsically express P-gp. In others, such expression can arise through spontaneous mutation or by dominant selection and growth of such cells, after exposure to pharmaceutical agents. Some pharmaceutical agents bind to or are substrates for P-gp, which is an indication that P-gp is likely to cause efflux of such agents from cells. Pharmaceutical agents that are taxane analogues can also be expected to be expelled from cells by P-gp. Other pharmaceutical agents (including other inhibitors of tubulin disassembly) may be unaffected by the presence of P-gp.

Extensive efforts have been focused on development of inhibitors of MDR and methods for their use. However, among the continuing drawbacks to known inhibitors and prescribed protocols for their use are the following:

(i) conventional inhibitors of MDR generally cause pharmacokinetic interactions with the co-administered active pharmaceutical agents, leading to unacceptable side effects requiring the careful determination and use of reduced dosages of such active pharmaceutical agents that may not be sufficient to achieve the desired therapeutic or preventative result.

(ii) Although oral administration of active pharmaceutical agents and inhibitors of MDR is desirable in order to avoid the pain and inconvenience of parenteral administration, the prior art teaches (contrary to our own findings) that such agents that operate by inhibition of P-gp generally have narrow-spectrum activity and accordingly are effective only to facilitate the passage of specific, defined pharmaceutical agents into cells; additional agents to facilitate oral bioavailability of active pharmaceutical agents in multi-drug resistant cells, and to improve the oral bioavailability of active pharmaceutical agents in the absence of MDR, are needed.

(iii) The blood-brain barrier is heavily protected by P-gp-mediated efflux; effective inhibitors of MDR at such barrier are needed to enhance delivery of neurologic therapeutic agents to brain targets.

The prior art generally cautions that effective inhibition of P-gp-mediated efflux of a given active pharmaceutical agent requires careful attention to the toxicity to be expected from the resultantly increased delivery of the active pharmaceutical agent. Contrary to these teachings, we have surprisingly discovered that the compounds of Formula 1 provide protection to the subject mammal against the toxicity of the administered active pharmaceutical agent.

The prior art discloses the use of specific P-gp inhibitors unrelated to the compounds of Formula 1 for the purpose of enhancing bioavailability of active pharmaceutical agents. However, the prior art teaches (contrary to our own findings) that specific P-gp inhibitors are only effective in increasing the bioavailability of specific active pharmaceutical agents, generally teaches against the utility of other P-gp inhibitors for this purpose, indicates that the mechanism of efficacy of such inhibitors may instead be through competition for cytochrome P-450 (CYP) metabolism in the gut (in particular, CYP 3A), and fails to disclose or suggest the use of the compounds of Formula 1. Contrary to the prior art, the compounds of Formula 1 have been found to be highly effective in enhancing bioavailability of active pharmaceutical agents, and are applicable to increase the bioavailability of co-administered active pharmaceutical agents that (i) bind to or are substrates for P-gp, and I or (ii) are taxane analogues. In addition, the compounds of Formula 1 provide protection of the subject mammal against the inherent toxicity of such active pharmaceutical agents—facilitating delivery to the target cells of higher dosages of the active pharmaceutical agents, yet under conditions of reduced toxicity. Moreover, the compounds of Formula 1 achieve this effect without inhibition of cytochrome P-450 3A (CYP-3A).

The prior art discloses the use of compounds unrelated to the compounds of Formula 1 for the purpose of facilitating penetration of the blood-brain barrier. The prior art further teaches that specific P-gp inhibitors are only effective in increasing bioavailability of specific active pharmaceutical agents. Contrary to the prior art, the compounds of Formula 1 have been found to be applicable to facilitating penetration across the blood-brain barrier by co-administered active pharmaceutical agents that (i) bind to or are substrates for P-gp, and/or (ii) are taxane analogues. In addition, the compounds of Formula 1 further provide protection of the subject mammal against the inherent toxicity of such active pharmaceutical agents, facilitating delivery to the target cells of high dosages of the active pharmaceutical agents under conditions of reduced toxicity.

Referring specifically to oncology, there are three major types of treatments currently in use against neoplasms: surgery, radiation therapy, and chemotherapy. Cytotoxic chemotherapeutic agents include a variety of natural products, for example taxanes, such as paclitaxel and docetaxel; vinca alkaloids such as vinblastine, vincristine and vinorelbine; anthracyclines such as doxorubicin and daunorubicin; and epipodophyllotoxins such as etoposide. The ability of these agents to cure neoplastic disease is extremely limited due to lack of tumor cell specificity, the presence of MDR tumor cells at the time of first diagnosis and the de novo emergence of MDR tumor cells during treatment.

Cytotoxic chemotherapeutic agents frequently suppress lymphocyte and hematopoietic and stem cell production, destroy the normal cells lining the digestive tract, and are toxic to the cardiovascular and nervous systems. These dose-limiting toxicities usually prevent the use of cytotoxic agents at doses that could kill sufficient numbers of tumor cells to effect a cure. The use of taxanes, vinca alkaloids, and anthracyclines is also largely limited to parenteral routes of administration, due to lack of oral bioavailability. This is due, in part, to the normal expression of P-gp in intestinal epithelial cells.

The most common mechanism of MDR in tumor cells involves the aberrant over-expression of P-gp, resulting in transport of chemotherapeutic agents out of the tumor cells before they can kill the cell. P-gp can bind and transport many pharmaceutical agents including taxanes, vinca alkaloids, anthracyclines and epipodophyllotoxins, to name a few, and its expression is sufficient to produce the MDR phenotype. P-gp expression has been found in many major tumor types at the time of first diagnosis, including acute myelogenous leukemia, breast cancer, ovarian cancer and colorectal carcinoma. In addition, P-gp expression in these tumor types increases after treatment of subjects with cytotoxic agents, via selection of pre-existing P-gp positive cells or spontaneous mutants expressing P-gp. Expression of P-gp in treated subjects contributes directly to therapeutic failure and relapse. For example, one study found that subjects with breast tumors expressing P-gp were three times more likely not to respond to chemotherapy than subjects whose tumors were P-gp negative. Drugs of proven antitumor chemotherapeutic value to which MDR has been observed include, for example, vinblastine, vincristine, etoposide, doxorubicin (adriamycin), daunorubicin, taxanes, plicamycin (mithramycin), and dactinomycin (Jones et al., Cancer (Suppl) 1993, 72:3484-3488). Many tumors are intrinsically multidrug resistant (e.g., adenocarcinomas of the colon and kidney) while other tumors acquire MDR during the course of therapy (e.g., neuroblastomas and childhood leukemias).

Various agents have been described that inhibit P-gp and which may be used with cytotoxic chemotherapeutics in relapsed or refractory disease. Most of these agents exhibit intrinsic cytotoxicity and alter the pharmacokinetics of the co-administered cytotoxic agent, forcing a significant reduction in the amount of cytotoxic drug that can be administered. These properties are not readily compatible with use in newly diagnosed or therapy naive subjects, even though this is the setting in which P-gp inhibition may be most effective.

### SUMMARY OF THE INVENTION

The present invention overcomes many of the problems discussed above by providing methods and compositions incorporating active pharmaceutical agents together with compounds having the following general structure, as fully defined later in the detailed description:

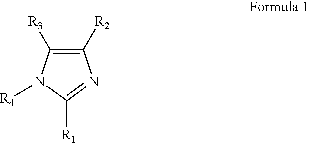

In preferred embodiments, such methods and compositions are provided in which the compound of Formula 1 is selected from: (2-[4-(3-ethoxy-1-propenyl)phenyl]-4,5-bis(4-(2-propylamino)phenyl)-1H-imidazole; 2-[4-(3-ethoxy-trans-1-propen-1-yl)phenyl]-4,5-bis (4-N,N-diethylaminophenyl) imidazole; 2-[4-(3-ethoxy-trans-1-propen-1-yl)phenyl]-4-(4-N,N-diethylaminophenyl)-5-(4-N-methylaminophenyl) imidazole; 2-[4-(3-methoxy-trans-1-propen-1-yl)phenyl]-4,5-bis (4-pyrrolidinophenyl) imidazole; 2-[4-(3-ethoxy-trans-1-propen-1-yl)phenyl]-4,5-bis (4-pyrrolidinophenyl) imidazole; 2-[4-(3-ethoxy-trans-1-propen-1-yl)phenyl]-4-(4-N-dimethylaminophenyl)-5-(4-pyrrolidinophenyl) imidazole; 2-[4-(3-ethoxy-trans-1-propen-1-yl)phenyl]-4-(4-N-methylaminophenyl)-5-(4-pyrrolidino-phenyl) imidazole; 2-[4-(3-ethoxy-trans-1-propen-1-yl)phenyl]-4,5-bis (4-N-morpholinophenyl) imidazole; 2-[4-(3-ethoxy-trans-1-propen-1-yl)phenyl]-4-(4-N-dimethylaminophenyl)-5-(4-N-morpholinophenyl) imidazole; 2-[4-(3-ethoxy-trans-1-propen-1-yl)phenyl]-4-(4-N-methylaminophenyl)-5-(4-N-morpholinophenyl) imidazole; and 2-[4-(3-ethoxy-trans-1-propen-1-yl)phenyl]-4-(4-N-methylaminophenyl)-5-(4-N-isopropylaminophenyl) imidazole.

In further preferred embodiments, such methods and compositions are provided in which the compound of Formula 1 is:

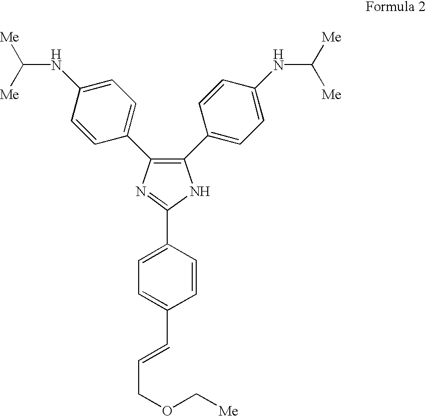

The methods and compositions of this invention are capable of protecting a mammal against the toxicity of active pharmaceutical agents so that otherwise toxic dosages of such active pharmaceutical agents having increased therapeutic and preventative efficacy can be used. Such methods and compositions are also capable of increasing the oral bioavailability of active pharmaceutical agents. These methods and compositions are further capable of facilitating the delivery of such active pharmaceutical agents in therapeutically and preventatively effective amounts across the blood-brain barrier.

In one embodiment, the invention provides a method of increasing the amount of a chemotherapeutic agent that can be safely administered to a subject. The method includes administering to a subject a compound having Formula 1 and a chemotherapeutic agent at a dose equal to or above standard levels, taking advantage of the protection provided to the subject by the compound of Formula I against toxicity of the chemotherapeutic agent.

In another embodiment, the invention provides a method of treating a subject having a cell proliferative disorder including orally administering to a subject an effective amount of a chemotherapeutic agent and an effective amount of a compound having Formula 1, taking advantage of the enhanced oral bioavailability of the chemotherapeutic agent provided to the subject by the compound of Formula 1 and thereby treating the subject.

In yet another embodiment, the invention provides a method of delivering a chemotherapeutic agent across the blood-brain barrier, taking advantage of the suppression of MDR to the chemotherapeutic agent provided to the subject by the compound of Formula 1.

### BRIEF DESCRIPTION OF THE FIGURES

FIG. 1 is a graph showing the effect of a preferred compound of Formula 2 on MDA/LCC6 and P-gp expressing MDA/LCC6^{MDRI} human breast carcinoma in response to paclitaxel in vitro.

FIG. 2 is a graph showing the effect of a preferred compound of Formula 2 on non-P-gp-expressing MDA/LCC6 human breast carcinoma xenografts in SCID mice in vivo.

FIG. 3 is a graph showing the effect of a preferred compound of Formula 2 on the toxicity and effect of paclitaxel in SCID mice in vivo (six mice per group).

FIG. 4 is a linear graphical representation of the concentration of paclitaxel in plasma vs. time, for the combination of paclitaxel and a preferred compound of Formula 2 in the form of a mesylate salt.

FIG. 5 is a graph demonstrating that pretreatment of mice with three oral doses of 30 mg/kg of a preferred Formula 2 compound had no effect on i.v. plasma paclitaxel levels.

FIG. 6 is a graph showing the elapsed time (latency in seconds) until mice escaped by jumping off a hot plate plotted versus elapsed time after administration of loperamide.

FIG. 7 is a linear graphical representation of the concentration of saquinavir in plasma vs. time.

FIG. 8 is a semi-log graphical representation of the concentration of saquinavir in plasma vs. time.

### DETAILED DESCRIPTION OF THE INVENTION

The compounds of Formula 1 operate to inhibit P-gp and accordingly reduce or prevent the development of MDR. We have discovered that the compounds of Formula 1 have activity as P-gp inhibitors when co-administered with active pharmaceutical agents that are selected from agents that: (i) bind to or are substrates for P-gp, and/or (ii) are taxane analogues. Such active pharmaceutical agents can be expected to otherwise be expelled by P-gp from cells intended to be treated by the active pharmaceutical agent. The compounds of Formula 1 also operate to facilitate protection against the toxicity of active pharmaceutical agents that are selected from agents that: (i) bind to or are substrates for P-gp, (ii) are taxane analogues and/or (iii) are inhibitors of tubulin disassembly

Active pharmaceutical compounds that (i) bind to or are substrates for P-gp, (ii) are taxane analogues, and/or (iii) are inhibitors of tubulin disassembly, can be categorized into the following classes of agents: taxanes, epothilones, discodermolide, eleutherobin, sarcodictyins, laulimalides, vinca alkaloids, anthracyclines, camptothecins, epipodophyllotoxins, methotrexate, angiotensin converting enzyme (ACE) inhibitors, human immunodeficiency virus protease inhibitors, antibiotics, calcium channel antagonists, β-blockers, HMG-CoA reductase inhibitors, immunosuppressive agents, opiates, fluoroquinolones, macrolide antibiotics, aminoglycoside antibiotics, antihistamines, anti-epileptic agents, anti-malarial agents, and dopamine agonists. A given compound can be used in a variety of forms, including a pharmaceutically-acceptable pro-drug, metabolite, analogue, derivative, solvate or salt.

Specific active pharmaceutical compounds that (i) bind to or are substrates for P-gp, (ii) are taxane analogues, and/or (iii) are inhibitors of tubulin disassembly, are subjects of ongoing research and identification. Such active pharmaceutical compounds that have been confirmed at least by some researchers to be within these classes of compounds include: Abeta1-40 (β-amyloid); Abeta1-42 (β-amyloid); Acebutolol; Dactinomycin; Adefovir; Adrenaline; Epinephrine; Albuterol; Salbutamol; Aldosterone; Amikacin; Amitriptyline; Amprenavir; Astemizole; Atorvastatin; Aureobasidin A; Azasetron; Azathioprine; Azidopine; Azithromycin; Bilirubin; Bisantrene; Bunitrolol; Burroughs Wellcome (“BW”) 1019W91; BW 1288U89; BW 1351W91; BW 1379W91; Calcein-AM; Carbamazepine; Carvedilol; Celiprolol; Cerivastatin; Chloroquine; Chlorpromazine; Cimetidine; Clarithromycin; Colchicine; Corticosterone; Cyclosporine; Cyclosporine metabolite AM1; Cytosine arabinoside (cytarabine); Daunorubicin; Debrisoquine; 13-OH-4′-Deoxy-4′-iododoxorubicin; Dexamethasone; Digitoxin; Digoxin; αMethyl-Digoxin; β-acetyl Digoxin; Dihydroindolizino[7,6,5-kl]acridinium chloride; Diltiazem; desacetyl Diltiazem; Dipyridamole; Docetaxel; Domperidone; Doxorubicin; DPDE [D-penicillamine(2,5)]-enkephalin]; D-Penicillamine; Ebastine; Eletriptan; Emetine; Epirubicin; Erythromycin; Estradiol-17-β-D-glucuronide; Etoposide; Felodipine; Fentanyl; Fexofenadine; Flavopiridol; Fluconazole; Fluvastatin; Furosemide; Gemtuzumab ozogamicin; Glibenclamide; Glyburide; Gramicidin D; Grepafloxacin; Hoechst 33342; Hydrocortisone (cortisol); Bayer BAY59-8862 (Indena IDN-5109 paclitaxel analog); Imatinib (Gleevec); Interleukin-2; Interleukin-4; Indinavir; Interferon 2B; Interferon-γ-1B; Irinotecan (CPT-11); Isoniazid; Ivermectin; Labetalol; Dilevalol; L-Dopa (levodopa); Levofloxacin; Loperamide; Loratadine; Losartan; Lovastatin; Mefloquine; Melphalan; Methadone; Methamphetamine; Methotrexate; Methylprednisolone; Mibefradil; Miltefosine; Mitomycin C; Mitoxantrone; Monensin; Morphine; Morphine-6-glucuronide; Moxidectin; MPP+ (1-Methyl-4-phenylpyridium); Nadolol; Naringin; Nelfinavir; Neostigmine; Nicardipine; Nonylphenol ethoxylate; Nortriptyline; Octreotide; Omeprazole; Ondansetron; Paclitaxel; Phenytoin; 2-amino-1-methyl-6-phenylimidazo[4,5-b]pyridine (PhlP); Phosphatidylcholine; Phosphatidylethanolamine; Pirarubicin; Platelet Activating Factor; Plicamycin (Mithramycin); Prazosin; Pristinamycins; Propantheline; Propranolol; PSC833; Puromycin; Quinidine; Quinine; Ranitidine; Reserpine; Retinoic acid; Ritonavir; Saquinavir; Simvastatin; Sirolimus; Somatropin; Sparfloxacin; Tacrolimus; Talinol; Tc-Sestamibi; Terfenadine; Tetracycline; Thapsigargin; Timolol; Tobramycin; Topotecan; Trimethoprim; UK-224,671; Vecuronium; Verapamil; Verapamil metabolite (D-617); Verapamil metabolite (D-620); Vinblastine; Vincristine; Vindesine; and Vinorelbine.

Preferred embodiments of the invention are directed to cytotoxic agents. Cytotoxic agents are commonly used as antineoplasm chemotherapeutic agents. These agents are also called antiproliferative agents and chemotherapeutic agents. The desired effect of cytotoxic drugs is selective cell death with destruction of the malignant neoplastic cells while sparing normal cells.

Cytotoxic agents have also proved valuable in the treatment of other neoplastic disorders including connective or autoimmune diseases, metabolic disorders, dermatological diseases, and viral infections.

Proper use of cytotoxic agents requires a thorough familiarity with the natural history and pathophysiology of the disease before selecting the cytotoxic agent, determining a dose, and undertaking therapy. Each subject must be carefully evaluated, with attention directed toward factors that may potentiate toxicity, such as overt or occult infections, bleeding dyscrasias, poor nutritional status, and metabolic disturbances. In addition, assessing the functional condition of certain major organs, such as the liver, kidneys, and bone marrow, is extremely important. Therefore, the selection of the appropriate cytotoxic agent and devising an effective therapeutic regimen is influenced by the presentation of the subject. Such considerations affect the dosage and type of drug administered.

Cytotoxic drugs as chemotherapeutic agents that (i) bind to or are substrates for P-gp, (ii) are taxane analogues, and/or (iii) are inhibitors of tubulin disassembly, can be subdivided into several broad categories, including,. taxanes, epothilones, discodermolide, eleutherobin, sarcodictyins, laulimalides, vinca alkaloids, anthracyclines, camptothecins, and epipodophyllotoxins. A given compound can be used in a variety of forms, including a pharmaceutically-acceptable pro-drug, metabolite, analogue, derivative, solvate or salt.

Specific chemotherapeutic compounds that (i) bind to or are substrates for P-gp, (ii) are taxane analogues, and/or (iii) are inhibitors of tubulin disassembly, are subjects of ongoing research and identification. Such chemotherapeutic compounds that have been confirmed at least by some researchers to be within these classes of compounds include: paclitaxel, docetaxel, vinblastine, vincristine, vinorelbine, doxorubicin, daunorubicin, etoposide, topotecan, dactinomycin, plicamycin (mithramycin), mitomycin, verapamil, cytosine arabinoside (cytarabine), methotrexate, and irinotecan (CPT-11). A given compound can be used in a variety of forms, including a pharmaceutically-acceptable pro-drug, metabolite, analogue, derivative, solvate or salt.

Important specific antitumor chemotherapeutic agents (with the usual effective dosage) to which clinical MDR has been observed include vinblastine (0.1 mg per kilogram per week), vincristine (0.01 mg per kilogram per week), etoposide (35 to 50 mg per square meter per day), dactinomycin (0.15 mg per kilogram per day), doxorubicin (500 to 600 mg per square meter per week), daunorubicin (65 to 75 mg per square meter per week), and mithramycin (0.025 mg per kilogram per day). MDR has been shown to occur in vitro as well as in the clinic. Accordingly, by increasing the dosage of these drugs, one can increase the cytotoxic effect upon the tumor cells. The present invention provides methods whereby cytotoxic drug dosage can be increased to otherwise toxic levels without increasing the toxicity to the subject.

The substituted imidazoles of the methods and compositions of the invention having the general Formula:

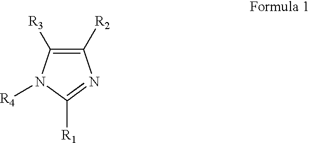

wherein the substituents R₁, R₂, R₃, and R₄ are defined as described in A and B below:

A. when R₁ is selected from the group consisting of:

(i) substituted C₁₋₁₁alkyl or substituted C₂₋₁₁alkenyl, wherein the substituents are selected from the group consisting of hydroxy, C₁₋₆alkyloxy; or

(ii) mono-, di-,and tri-substituted aryl-C₀₋₁₁ alkyl wherein aryl is selected from the group consisting of phenyl, furyl, thienyl wherein the substituents are selected from the group consisting of:

(a) phenyl, trans-2-phenylethenyl, 2-phenylethynyl, 2-phenylethyl, or in which the said phenyl group is mono- or disubstituted with a member selected from the group consisting of hydroxy, halo, C₁₋₄alkyl and C₁₋₄alkyloxy,

(b) substituted C₁₋₆alkyl, substituted C₂₋₆alkyloxy, substituted C₂₋₆alkylthio, substituted C₂₋₆alkoxycarbonyl, wherein the substituents are selected from the group consisting of C₁₋₆alkoxy, C₁₋₆alkylthio, or

(c) C₁₋₁₁CO₂R₅, C₁₋₁₁CONHR₅, trans-CH═CHCO₂R₅, or trans-CH═CHCONHR₅ wherein R₅ is C₁₋₁₁alkyl, or phenyl C₁₋₁₁alkyl, C₁₋₆alkoxycarbonylmethyleneoxy;

then R₂ and R₃ are each independently selected from the group consisting of mono-, di, and tri-substituted phenyl wherein the substituents are independently selected from:

(i) substituted C₁₋₆alkyl,

(ii) substituted C₁₋₆alkyloxy, C₃₋₆alkenyloxy, substituted C₃₋₆alkenyloxy,

(iii) substituted C₁₋₆alkyl-amino, di(substituted C₁₋₄alkyl)amino,

(iv) C₃₋₆alkenyl-amino, di(C₃₋₆alkenyl)amino, substituted C₃₋₆alkenyl-amino, di(substituted C₃₋₆alkenyl)amino,

(v) pyrrolidino, piperidino, morpholino, imidazolyl, substituted imidazolyl, piperazino, N—C₁₋₆alkylpiperazino, N—C₃₋₆alkenylpiperazino, N—(C₁₋₆alkoxy C₁₋₆alkyl)piperazino, N—(C₁₋₆alkoxy C₃₋₆alkenyl)piperazino, N—(C₁₋₆alkylamino C₁₋₆alkyl)piperazino, N—(C₁₋₆alkylamino C₃₋₆alkenyl)piperazino,

wherein the substituents are selected from the group consisting of:

(a) hydroxy, C₁₋₆alkylalkoxy, C₁₋₆alkylamino,

(b) C₃₋₆alkenyloxy, C₃₋₆alkenylamino, or

(c) pyrrolidino, piperidino, morpholino, imidazolyl, substituted imidazolyl, piperazino, N—C₁₋₆alkylpiperazino, N—C₃₋₆alkenylpiperazino, N—(C₁₋₆alkoxy C₁₋₆alkyl)piperazino, N—(C₁₋₆alkoxy C₃₋₆alkenyl)piperazino, N—(C₁₋₆alkylamino C₁₋₆alkyl)piperazino, N—(C₁₋₆alkylamino C₃₋₆alkenyl)piperazino,

or R₂ and R₃ taken together forming an aryl group or substituted aryl, wherein the substituents are defined as above in (i)-(v);

and R₄ is selected from the group consisting of:

(i) hydrogen;

(ii) substituted C₁₋₁₁alkyl or C₂₋₁₁alkenyl wherein the substituents are independently selected from the group consisting of hydrogen, hydroxy, C₁₋₆alkyloxy, C₁₋₆alkylthio, C₁₋₆alkylamino, phenyl-C₁₋₆alkylamino, C₁₋₆alkoxycarbonyl; or

(iii) substituted aryl C₀₋₁₁alkyl wherein the aryl group is selected from phenyl, imidazolyl, furyl, thienyl in which the substituents are selected from A.(a-c); or

B. when R₁ is selected from the group consisting of:

Mono-,di-, and tri-substituted aryl-C₀₋₆alkyl wherein aryl is selected from the group consisting of phenyl, thienyl, and the substituents are selected from the group consisting of:

(a) trans-2-substituted benzimidazolylethenyl, trans-2-substituted benzoxazolylethenyl, trans-2-substituted benzthiazolylethenyl, in which the substituents are selected from the group consisting of hydrogen, hydroxy, halo, trihalomethyl, C₁₋₄alkyl and C₁₋₄alkyloxy, C₁₋₄alkyloxycarbonyl, C₁₋₄alkylamino, di(C₁₋₄alkyl)amino, C₃₋₆alkenylamino, di(C₃₋₆alkenyl)amino, C₁₋₄alkyloxy-C₁₋₄alkylamino, substituted C₁₋₄alkyl and C₁₋₄alkyloxy, substituted C₁₋₄alkyloxycarbonyl, substituted C₁₋₄alkylamino, di(substituted C₁₋₄alkyl)amino, substituted C₃₋₆alkenylamino, di(substituted C₃₋₆alkenyl)amino, wherein the substituents are as defined above,

(b) trans-2-cyano ethenyl, trans-2-alkylsulfonyl ethenyl, trans-2-alkenylsulfonyl ethenyl, trans-2-substituted alkylsulfonyl ethenyl, trans-2-substituted alkenylsulfonyl ethenyl, in which the substituents are defined above,

(c) C₁₋₆CO₂R₅, trans-CH═CHCO₂R₅, C₁₋₆CONHR₅, or trans-CH═CHCONHR₅, wherein R₅ is C₁₋₆alkoxy C₂₋₆alkyl, amino C₂₋₆alkyl, C₁₋₆alkylamino C₂₋₆alkyl, di(C₁₋₆alkyl)amino C₂₋₆alkyl, C₁₋₆alkylthio C₂₋₆alkyl, substituted C₁₋₆alkoxy C₂₋₆alkyl, substituted C₁₋₆alkylamino C₂₋₆alkyl, di(substituted C₁₋₆alkyl)amino C₂₋₆alkyl, substituted C₁₋₆alkylthio C₂₋₆alkyl, in which the substituents are selected from the group consisting of pyrrolidino, piperidino, morpholino, piperazino, N—C₁₋₆alkylpiperazino, N—C₃₋₆alkenylpiperazino, N—(C₁₋₆alkoxy C₁₋₆alkyl)piperazino, N—(C₁₋₆alkoxy C₃₋₆ alkenyl)piperazino, N—(C₁₋₆alkylamino C₁₋₆alkyl)piperazino, N—(C₁₋₆alkylamino C₃₋₆alkenyl)piperazino, imidazolyl, oxazolyl, thiazolyl,

(d) C₁₋₆CONR₆R₇, or trans-CH═CHCONR₆R₇, wherein R₆ and R₇ are independently selected from the group consisting of C₁₋₆alkyl, phenyl C₁₋₆alkyl, C₁₋₆alkoxycarbonylmethyleneoxy, hydroxy C₂₋₆alkyl, C₁₋₆alkyloxy C₂₋₆alkyl, amino C₂₋₆alkyl, C₁₋₆alkylamino C₂₋₆alkyl, di(C₁₋₆alkyl)amino C₂₋₆alkyl, C₁₋₆alkylthio C₂₋₆alkyl, substituted C₁₋₆alkoxy C₂₋₆alkyl, substituted C₁₋₆alkylamino C₂₋₆ alkyl, di(substituted C₁₋₆ alkyl)amino C₂₋₆alkyl, substituted C₁₋₆alkylthio C₂₋₆alkyl, wherein the substituents are selected from the group consisting of pyrrolidino, piperidino, morpholino, piperazino, N—(C₁₋₆alkylpiperazino, N—(C₃₋₆alkenylpiperazino, N—(C₁₋₆alkoxy C₁₋₆alkyl)piperazino, N—(C₁₋₆alkoxy C₃₋₆alkenyl)piperazino, N—(C₁₋₆alkylamino C₁₋₆alkyl)piperazino, N—(C₁₋₆alkylamino C₃₋₆alkenyl)piperazino, imidazolyl, oxazolyl, thiazolyl,

(e) R₇ C(O) C₁₋₆alkyl, R₇C(O) carbonyl C₂₋₆alkenyl, in which R₇ is defined as above [2(d)],

(f) HO—C₁₋₆alkyl-C₂₋₆alkenyl, R₇—O—C₁₋₆alkyl-C₂₋₆alkenyl, R₇NH—C₁₋₆alkyl-C₂₋₆alkenyl, R₆R₇N—C₁₋₆alkyl-C₂₋₆alkenyl, R₇NH—C(O)—O—C₁₋₆alkyl-C₂₋₆alkenyl, R₆R₇N—C(O)—O—C₁₋₆alkyl-C₂₋₆alkenyl, R₇O—C(O)—O—C₁₋₆alkyl-C₂₋₆alkenyl, R₇—C(O)—O—C₁₋₆alkyl-C₂₋₆alkenyl, wherein R₆ and R₇ is defined as above [2(d)],

(g) R₇—O—C₀₋₃alkyl-C₃₋₆cycloalkan-1-yl, R₇NH—C₀₋₃alkyl-C₃₋₆cycloalkan-1-yl, R₆R₇N—C₀₋₃alkyl-C₃₋₆cycloalkan-1-yl, R₇NH—C(O)—O—C₀₋₃C₃₋₆cycloalkan-1-yl, R₆R₇N—C(O)—O—C₀₋₃alkyl-C₃₋₆cycloalkan-1-yl, R₇O—C(O)—O—C₀₋₃alkyl-C₃₋₆cycloalkan-1-yl, R₇—C(O)—O—C₀₋₃alkyl-C₃₋₆cycloalkan-1-yl, R₇O—C(O)-C₀₋₃alkyl-C₃₋₆cycloalkan-1-yl, wherein R₇ and is defined as above [2(d)];

then R₂ and R₃ are each independently selected from the group consisting of:

(1) hydrogen, halo, trihalomethyl, C₁₋₆alkyl, substituted C₁₋₆alkyl, C₁₋₆alkenyl, substituted C₁₋₆alkenyl, C₁₋₆alkyloxy, substituted C₁₋₆alkyloxy, C₃₋₆alkenyloxy, substituted C₃₋₆alkenyloxy, C₁₋₆alkylamino, substituted C₁₋₆alkylamino, C₃₋₆alkenylamino, substituted C₃₋₆alkenylamino,

(2) mono-, di-, and tri-substituted phenyl wherein the substituents are independently selected from:

(i) halo, trifluoromethyl, substituted C₁₋₆alkyl,

(ii) C₁₋₆alkyloxy, substituted C₁₋₆alkyloxy, C₃₋₆alkenyloxy, substituted C₃₋₆alkenyloxy,

(iii) C₁₋₆alkyl-amino, di(C₁₋₆alkyl)amino, substituted C₁₋₆alkyl-amino, di(substituted C₁₋₆alkyl)amino, C₃₋₆alkenyl-amino, di(C₃₋₆alkenyl)amino, substituted C₃₋₆alkenyl-amino, di(substituted C₃₋₆alkenyl)amino, or

(iv) pyrrolidino, piperidino, morpholino, imidazolyl, substituted imidazolyl, piperazino, N—C₁₋₆alkylpiperazino, N—C₃₋₆alkenylpiperazino, N—(C₁₋₆alkoxy C₁₋₆alkyl)piperazino, N—(C₁₋₆alkoxy C₃₋₆alkenyl)piperazino, N—(C₁₋₆alkylamino C₁₋₆alkyl)piperazino, N—(C₁₋₆alkylamino C₃₋₆alkenyl)piperazino,

wherein the substituents are selected from the group consisting of

(a) hydrogen, hydroxy, halo, trifluoromethyl,

(b) C₁₋₆alkylalkoxy, C₁₋₆alkylamino, C₁₋₆alkylthio,

(c) C₃₋₆alkenyloxy, C₃₋₆alkenylamino, C₃₋₆alkenylthio, or

(d) pyrrolidino, piperidino, morpholino, imidazolyl, substituted imidazolyl, piperazino, N—C₁₋₆alkylpiperazino, N—C₃₋₆alkenylpiperazino, N—(C₁₋₆alkoxy C₁₋₆alkyl)piperazino, N—(C₁₋₆alkoxy C₃₋₆alkenyl)piperazino, N—(C₁₋₆alkylamino C₁₋₆alkyl)piperazino, N—(C₁₋₆alkylamino C₃₋₆alkenyl)piperazino;

with the proviso that at least one of R₂ and R₃ group be selected from

[B (2)] and the phenyl and the substituents be selected from (ii)-(v) above; or R₂ and R₃ taken together forming an aryl group or substituted aryl, wherein the substituents are defined as above in (i)-(iv);

and R₄ is selected from the group consisting of:

(a) hydrogen;

(b) substituted C₁₋₁₁alkyl or C₂₋₁₁alkenyl wherein the substituents are independently selected from the group consisting of hydrogen, hydroxy, C₁₋₆alkyloxy, C₁₋₆alkylthio, C₁₋₆alkylamino, phenyl-C₁₋₆alkylamino, C₁₋₆alkoxycarbonyl and the substituents are selected from (ii)-(iv); or

(c) aryl C₀₋₁₁alkyl wherein the aryl group is selected from phenyl, imidazolyl, furyl, thienyl

have been shown to inhibit P-gp functionality and are effective in reversing MDR in cells that express P-gp (see U.S. Pat. Nos. 5,700,826; 5,756,527; and 5,840,721, which are incorporated herein by reference in their entirety with particular attention directed to U.S. Pat. No. 5,840,721 at column 10, lines 50-62 and column 62, lines 31-62). The compounds of Formula 1 can be prepared by procedures known to those skilled in the art from known compounds or readily preparable intermediates (see, for example, U.S. Pat. No. 5,840,721 beginning, e.g., at column 10, lines 50-62).

Of the compounds encompassed by Formula 1, the following compounds are preferred: (2-[4-(3-ethoxy-1-propenyl)phenyl]-4,5-bis(4-(2-propylamino)phenyl)-1H-imidazole; 2-[4-(3-ethoxy-trans-1-propen-1-yl)phenyl]-4,5-bis (4-N,N-diethylaminophenyl) imidazole; 2-[4-(3-ethoxy-trans-1-propen-1-yl)phenyl]-4-(4-N,N-diethylaminophenyl)-5-(4-N-methylaminophenyl) imidazole; 2-[4-(3-methoxy-trans-1-propen-1-yl)phenyl]-4,5-bis (4-pyrrolidinophenyl) imidazole; 2-[4-(3-ethoxy-trans-1-propen-1-yl)phenyl]-4,5-bis (4-pyrrolidinophenyl) imidazole; 2-[4-(3-ethoxy-trans-1-propen-1-yl)phenyl]-4-(4-N-dimethylaminophenyl)-5-(4-pyrrolidinophenyl) imidazole; 2-[4-(3-ethoxy-trans-1-propen-1-yl)phenyl]-4-(4-N-methylaminophenyl)-5-(4-pyrrolidino-phenyl) imidazole; 2-[4-(3-ethoxy-trans-1-propen-1-yl)phenyl]-4,5-bis (4-N-morpholinophenyl) imidazole; 2-[4-(3-ethoxy-trans-1-propen-1-yl)phenyl]-4-(4-N-dimethylaminophenyl)-5-(4-N-morpholinophenyl) imidazole; 2-[4-(3ethoxy-trans-1-propen-1-yl)phenyl]-4-(4-N-methylaminophenyl)-5-(4-N-morpholinophenyl) imidazole; and 2-[4-(3-ethoxy-trans-1-propen-1-yl)phenyl]-4-(4-N-methylaminophenyl)-5-(4-N-isopropylaminophenyl) imidazole.

In further preferred embodiments, the compound of Formula 1 is (2-[4-(3-ethoxy-1-propenyl)phenyl]-4,5-bis(4-(2-propylamino)phenyl)-1H-imidazole, as depicted in Formula 2:

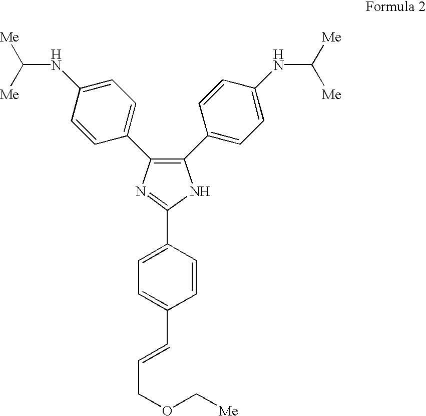

In preferred embodiments, this compound is in the form of a mesylate salt.

The present invention is based upon the discoveries that the compounds of Formula 1, as free compounds, or in the form of pharmaceutically-acceptable pro-drugs, metabolites, analogues, derivatives, solvates or salts, not only inhibit MDR in tumor cells expressing P-gp as described, e.g., in U.S. Pat. Nos. 5,700,826; 5,756,527; and 5,840,721, but also: (1) inhibit MDR in other types of cells expressing P-gp; (2) allow the safe administration of selected pharmaceutical and chemotherapeutic agents to treat subjects at standard or even higher doses thought to be toxic, particularly subjects that are naive to pharmaceutical or chemotherapeutic treatment; (3) increase the bioavailability of orally administered active pharmaceutical agents that (i) bind to or are substrates for P-gp, and/or (ii) are taxane analogues; and (4) facilitate the penetration of such active pharmaceutical agents across the blood-brain barrier. In vivo administration of a compound of Formula 1 in combination with pharmaceutical or chemotherapeutic agents also enhances the therapeutic effect of such pharmaceutical or cytotoxic agents against cells that do not express P-gp, thus preventing the subsequent emergence of MDR.

The invention is particularly useful for the administration of taxanes in chemotherapy. In these embodiments, the active pharmaceutical agent is a chemotherapeutic compound comprising a taxane in the form of a free compound or its pharmaceutically-acceptable pro-drug, metabolite, analogue, derivative, solvate or salt. In one embodiment the taxane is paclitaxel. If parenteral administration of paclitaxel is desired, then the toxicity-protected dosage range of paclitaxel during a given treatment session is about 100 mg/m² to about 675 mg/m². In preferred practice for such parenteral administration, the toxicity-protected dosage of paclitaxel is about 350 mg/m² to about 675 mg/m². In the case of oral administration of paclitaxel, the toxicity-protected dosage of paclitaxel is about 125 mg to about 1200 mg per treatment session; preferably about 550 mg to about 1200 mg per treatment session.

In further embodiments, the taxane is docetaxel. If parenteral administration of docetaxel is desired, then the toxicity-protected dosage range of docetaxel during a given treatment session is about 100 mg/m² to about 675 mg/m². In preferred practice for such parenteral administration, the toxicity-protected dosage of docetaxel is about 350 mg/m² to about 675 mg/m². In the case of oral administration of docetaxel, the toxicity-protected dosage of docetaxel is about 125 mg to about 1200 mg per treatment session; preferably about 550 mg to about 1200 mg per treatment session.

The effective dosage for a given chemotherapeutic agent may be determined based upon its chemotherapeutic index (minimum toxic dose divided by minimum effective dose (LD50/ED50)). Treatment with a compound of Formula 1 allows the use of higher dosages of the anti-cell-proliferative therapeutic agent, increasing the chemotherapeutic index. (See, e.g., Goodman & Gillman's The Pharmacological Basis of Therapeutics,” 9th Ed., (McGraw Hill 1996), pp. 48-49.

In one preferred embodiment for parenteral administration of paclitaxel or docetaxel, a treatment regimen comprises administering: (a) about 35 mg to about 700 mg of the compound of Formula 1 at about 8 to about 16 hours before such paclitaxel or docetaxel administration; (b) about 35 mg to about 700 mg of the compound of Formula 1 at about 1 to about 3 hours before or with such paclitaxel or docetaxel administration; and (c) about 35 mg to about 700 mg of the compound of Formula 1 at about 6 to about 10 hours after such paclitaxel or docetaxel administration. In a further preferred embodiment, each treatment comprises administering: about 50 mg to about 500 mg of the compound of Formula 1.

In one preferred embodiment for oral administration of paclitaxel or docetaxel, a treatment regimen comprises administering: (a) about 100 mg to about 750 mg of the compound of Formula 1 at about 8 to about 16 hours before such paclitaxel or docetaxel administration; (b) about 100 mg to about 750 mg of the compound of Formula 1 at about 1 to about 3 hours before or with such paclitaxel or docetaxel administration; and (c) about 100 mg to about 750 mg of the compound of Formula 1 at about 6 to about 10 hours after such paclitaxel or docetaxel administration. In a further preferred embodiment, each treatment comprises administering: about 300 mg to about 500 mg of the compound of Formula 1.

In the case of paclitaxel and docetaxel, one standard regimen includes administration of the anti-cell-proliferative therapeutic agent at a frequency of about once every three weeks during a course of treatment. In accordance with the invention, this frequency can be increased to at least about once every two weeks during such course of treatment. Another standard paclitaxel and docetaxel regimen includes administration of the anti-cell-proliferative therapeutic agent at a frequency of about once every week during a course of treatment. In accordance with the invention, this frequency can be increased to at least about once every three days during such course of treatment.

Several embodiments specifically relate to compounds of Formula 2. One of these embodiments relates to the use of a compound of Formula 2 in treatment of neoplasms with a taxane. Neoplasms may include, as examples: cancer (including but not limited to breast cancer), tumors, fibrotic disorders, and acute myeloid leukemia. The compound of Formula 2 has been found to be non-cytotoxic against normal or tumor cell lines at doses up to 100 micromolar (“μM”) and did not enhance the cytotoxic effect of chemotherapeutics against cells which do not express P-gp, under in vitro conditions. In addition, when the compound of Formula 2 was tested in vivo with co-administration of natural product chemotherapeutic agents such as, for example, paclitaxel, the compound of Formula 2 had no intrinsic anti-tumor activity and did not enhance the toxicity of co-administered paclitaxel. However, the compound of Formula 2 enhanced the anti-tumor effect of cytotoxic drugs such as paclitaxel against human tumor xenografts that did not express P-gp. This unexpected synergy was not the result of an effect of the compound of Formula 2 on paclitaxel blood levels.

In another embodiment, the invention provides a method of reducing the cytotoxic effects of chemotherapeutic agents on non-cancer cells. Co-administration of the compound of Formula 2, in the form of a free compound, or a pharmaceutically-acceptable pro-drug, metabolite, analogue, derivative, solvate or salt thereof, with a chemotherapeutic agent (e.g., paclitaxel) reduced the toxicity of the chemotherapeutic agent to the subject. The compound of Formula 2 protected subjects from the lethal effects of high-dose chemotherapeutic therapy, such as paclitaxel therapy. Under these conditions, the compound of Formula 2 allows the administration of high enough doses of chemotherapeutic agents to completely inhibit the growth of tumors that do not express P-gp. Complete (100%) suppression of tumor growth was not observed with paclitaxel alone under any circumstances. The term “subject” as used herein refers to any mammal having a cell proliferative disorder (e.g., a neoplastic disorder). Subjects for the purposes of the invention include, but are not limited to, mammals (e.g., bovine, canine, equine, feline, porcine) and preferably humans.

In one embodiment broadly relating to compounds of Formula 1, the present invention provides a method for treating a subject having a cell proliferative disorder. The method includes administering a compound of Formula 1, in the form of a free compound or a pharmaceutically-acceptable pro-drug, metabolite, analogue, derivative, solvate or salt, prior to, simultaneously with, or subsequent to administration of a chemotherapeutic agent.

Preferably the subject is a naïve subject. A “naïve subject” is a subject that has not been treated with the same chemotherapeutic agent. As described more fully below, the compound of Formula 1, in the form of a free compound or a pharmaceutically-acceptable pro-drug, metabolite, analogue, derivative, solvate or salt (e.g., the mesylate salt) is administered. Preferably, the mesylate salt is administered.

By “cell proliferative disorder” is meant a cell or cells that demonstrate abnormal growth, typically aberrant growth, leading to a neoplasm, tumor or a cancer. Cell proliferative disorders include, for example, cancers of the breast, lung, prostate, kidney, skin tissue, central nervous system, ovary, uterus, liver, pancreas, adrenal gland, epithelial system, gastric system, intestinal system, exocrine system, endocrine system, lymphatic system, hematopoietic system, genitourinary system, colorectal system, or head and neck tissue. Preferably the cancer does not necessarily express P-gp. More generally, neoplastic diseases are conditions in which abnormal proliferation of cells results in a mass of tissue called a neoplasm or tumor. Neoplasms have varying degrees of abnormalities in structure and behavior. Some neoplasms are benign while others are malignant or cancerous. An effective treatment of neoplastic disease would be considered a valuable contribution to the search for cancer preventive or curative procedures.

For example, in one embodiment a compound of Formula 1, in the form of a free compound or a pharmaceutically-acceptable pro-drug, metabolite, analogue, derivative, solvate or salt, e.g., the mesylate salt, is administered to a subject who has not previously been exposed to chemotherapy and who has cells having a cell proliferative disorder which do not express P-gp. Under these conditions, administration of the compound of Formula 1, in the form of a free compound or a pharmaceutically-acceptable pro-drug, metabolite, analogue, derivative, solvate or salt, enhances the anti-tumor activity of a co-administered cytotoxic agent, such as a taxane. The dose and efficacy of the compound of Formula I and the chemotherapeutic agent would be in an effective amount to inhibit MDR in any sub-population of tumor cells expressing P-gp, and/or prevent the emergence of P-gp expressing cells. The methods of the invention allow the safe use of doses of chemotherapeutic agents that are, by themselves, often unacceptably toxic, the combination of which produces an increased remission rate. As used herein, an “effective amount” is that amount capable of inhibiting or modulating cell growth activity of neoplastic cells.

In another embodiment, the invention provides compositions and methods useful to protect a subject from the cytotoxic effects of chemotherapeutic agents. The method includes administering to a subject a protective effective amount of a compound having Formula 1, in the form of a free compound or a pharmaceutically-acceptable pro-drug, metabolite, analogue, derivative, solvate or salt. The compound of Formula 1 reduces the lethality of the chemotherapeutic agent to the subject. The compound of Formula 1 protected subjects from the lethal effects of high-dose chemotherapeutic therapy, such as, for example, paclitaxel therapy. Under these conditions, the compound of Formula 1 allows the administration of high enough doses of chemotherapeutic agents to completely inhibit the growth of tumors that do not express P-gp.

Under conditions where the compound of Formula 1, in the form of a free compound or a pharmaceutically-acceptable pro-drug, metabolite, analogue, derivative, solvate or salt is administered to protect a subject from chemotherapeutic agent toxicity, the method of treatment includes standard doses as well as, for example, an increase above each standard dose by about 25 to 100%, more preferably, about 50 to 100%. Thus, for example, paclitaxel is given as a 1-hour, 3-hour, or 24-hour intravenous infusion (i.e., a “treatment session”, this definition being applicable to other active pharmaceutical agents as well) at doses from 80 to 225 mg/m² either once per week, once every four days, or every three weeks. In conjunction with the compound of Formula 1, the paclitaxel dose could be increased, e.g., from approximately 1.25 to 3-fold, depending on the regimen.

In yet another embodiment, the invention provides a method of treatment or preventing growth of MDR or drug-resistant tumor cells by administering a sufficient amount of a compound of Formula 1, in the form of a free compound or a pharmaceutically-acceptable pro-drug, metabolite, analogue, derivative, solvate or salt, prior to, together with, or subsequent to the administration of an antitumor chemotherapeutic agent. Administration of the compound of Formula 1 and a chemotherapeutic agent results in the suppression of tumor growth by at least 50%; preferably 60%; and, more preferably, greater than 70%. Accordingly, the elimination of tumor growth and proliferation eliminates the production of MDR tumor cells reducing the recurrence of cancer and increasing the efficacy of chemotherapeutic treatments. The compositions and methods of the invention not only inhibit MDR in tumor cells expressing P-gp, but also allow the safe administration of chemotherapeutic agents at standard or even higher doses to treat naive subjects, enhancing the therapeutic effect of chemotherapeutic agents against tumors that do not express P-gp and simultaneously preventing the subsequent emergence of MDR. In the absence of the toxicity protection provided by the compounds of Formula 1 as used in accordance with the invention, naive patients frequently cannot be given therapeutically optimal initial dosages of the selected chemotherapeutic agent because of the agents dose limiting toxicity. Such reduced dosages encourage the development of MDR, defeating the treatment regimen. The methods and compositions of the invention make possible initial administration of otherwise toxic doses of the chemotherapeutic agent, with the real potential of not only preventing development of MDR, but of curing the cancer.

The methods and compositions of the invention are useful for increasing the sensitivity of cells having cell proliferative disorders (e.g., a neoplasm) to chemotherapeutic agents such as, for example, paclitaxel. By increasing the efficacy without concomitant toxicity to non-cancer cells the invention provides methods and compositions useful for treating tumors and preventing or reducing the chances of relapse and death as a result of cytotoxicity. In addition, the invention eliminates or reduces the number of MDR cells by eliminating cancer cells prior to any mutation inducing an MDR phenotype or overproduction of P-gp conferring an MDR phenotype. Accordingly, by reducing multi-drug resistant tumor cells from arising, the invention satisfies the shortcomings of current therapeutic modalities.

These methods are useful in treatment of cells that express P-gp and manifest MDR. In addition, these methods can be used in treatment of naive cells that have not previously been exposed to an anti-cell-proliferative therapeutic agent. Similarly, these methods can be applied to cells that do not express P-gp, do not express P-gp in all cells, or do not express P-gp at levels sufficient to manifest complete MDR. The toxicity protection provided by the compounds of Formula 1 permits usage of substantially greater dosages of the chemotherapeutic agents than could normally be employed, providing an improved opportunity to eliminate the disease rather than encourage development of MDR.

The compounds and methods of the invention are capable of sensitizing tumor cells to antitumor chemotherapeutic agents, such as taxanes regardless of the expression of P-gp. They also have the ability to potentiate the sensitivity of tumor cells susceptible to these chemotherapeutic agents. The invention also provides a method of sensitizing naïve or non-naïve and/or MDR tumor cells to antitumor chemotherapeutic agents. It also relates to a method of increasing the sensitivity of drug-susceptible tumor cells to antitumor chemotherapeutic agents. In addition, this invention relates to a method of inhibiting the emergence of MDR tumor cells during a course of treatment with antitumor chemotherapeutic agents.

According to further embodiments, the invention broadly provides methods for administering toxicity-protected dosages of pharmaceutical agents to a mammal. These methods include steps of:

(a) choosing a regimen of dosage frequency and amount of the pharmaceutically-active agent for such mammal that is therapeutically effective in the absence of the compound of Formula 1, taking into account the systemic toxicity of such pharmaceutically-active agent; and

(b) substantially increasing such dosage frequency or amount of the pharmaceutically-active agent to a toxicity-protected dosage, taking into account the protection against such systemic toxicity provided by such compound of Formula 1; and

(c) administering to such mammal (i) an effective amount of the compound of Formula 1 in the form of a free compound or its pharmaceutically-acceptable pro-drug, metabolite, analogue, derivative, solvate or salt; and (ii) such toxicity-protected dosage of such pharmaceutically-active agent.

These methods can be carried out by first choosing an active pharmaceutical agent that either (i) binds to or is a substrate for P-gp, (ii) is a taxane analogue, and/or (iii) is an inhibitor of tubulin disassembly. Next, the normal dosage regimen for the active pharmaceutical agent is determined, for example by reference to the Physician's Desk Reference. Consideration of the specific treatment indication relevant to the subject mammal, is typically part of this determination. Finally, the toxicity protection provided by the compound of Formula 1 to be co-administered, is relied upon to increase the dosage to a toxicity-protected level. Such dosages for particular active pharmaceutical agents can be determined through standard clinical trial procedures directed to toxicity assessment. In addition to enabling the administration of active pharmaceutical agents under regimens exceeding the maximum recommended dosage amounts and/or frequency for such active pharmaceutical agents, the methods of this invention further provide protection against the toxicity of standard dosages of such active pharmaceutical agents.

In preferred embodiments, the dosage amounts for such active pharmaceutical agents are increased by at least about 25% above the normal dosage regimen. In further preferred embodiments, such dosage amounts for such active pharmaceutical agents are increased by at least about 50% above the normal dosage regimen. In additional preferred embodiments, such dosage amounts for such active pharmaceutical agents are increased by at least about 100% above the normal dosage regimen. In other preferred embodiments, such dosage amounts for such active pharmaceutical agents are increased by about 50% to about 100% above the normal dosage regimen. In addition to increasing dosage amounts, the frequency of dosage deliveries can also be increased, either instead of or in addition to increasing the dosage amounts.

The active pharmaceutical agents and compounds of Formula 1 each can be delivered either by oral, parenteral, or topical means. The compound of Formula 1 can be administered either before, after, before and after, and/or simultaneously with the active pharmaceutical agent. As circumstances dictate, the active pharmaceutical agents and compounds of Formula 1 can be administered separately or in combined dosage forms. The toxicity protection of the invention can be particularly useful in cases where a chronic disease is expected to undergo long-term ongoing treatment with active pharmaceutical agents, such as treatment of neoplasms including cancer.

The methods of the invention can be applied to treatment of diseases of the following: an organ, including a: breast, lung, prostate, kidney, ovary, uterus, liver, pancreas, adrenal gland or; a system, including the epithelial, gastric, intestinal, exocrine, endocrine, lymphatic, hematopoietic, genitourinary, colorectal, or central nervous system, or; tissue, including: head, neck or skin tissue. Central nervous system diseases to be treated can include, without limitation: pain, epilepsy, cognitive disorders, Alzheimer's disease, and Parkinson's disease. In addition, the methods of the invention can be applied to treatment of infectious diseases, including viral, bacterial, fungal, and parasitic infections. One specific example of a viral infection is human immunodeficiency virus. Further, the methods of the invention can be applied to treatment of topical diseases, such as, for example, psoriasis. Also, the methods of the invention can be applied to treatment of mammals in which the disease is organ failure requiring an organ transplantation under conditions to prevent tissue rejection. In preferred embodiments, the mammal is a human.

The methods of this invention involve in one embodiment, (1) the administration of a compound of Formula 1, in the form of a free compound or a pharmaceutically-acceptable pro-drug, metabolite, analogue, derivative, solvate or salt, prior to, together with, or subsequent to the administration of an active pharmaceutical agent or a chemotherapeutic agent; or (2) the administration of a combination of a compound of Formula 1 and such an agent.

Thus, the compounds of Formula 1 in the form of a free compound or a pharmaceutically-acceptable pro-drug, metabolite, analogue, derivative, solvate or salt are useful in the treatment of MDR diseases in general, as well as neoplasms in particular, either separately or in combination with an active pharmaceutical or chemotherapeutic agent. These compounds may be administered orally, topically or parenterally in dosage unit formulations containing conventional non-toxic pharmaceutically acceptable carriers, adjuvants, and vehicles. The term parenteral as used herein includes subcutaneous injections, aerosol, intravenous, intramuscular, intrathecal, intracranial, intrasternal injection or infusion techniques.

The present invention also has the objective of providing suitable topical, oral, and parenteral pharmaceutical formulations for use in the novel methods of treatment of the present invention. The compounds of the present invention may be administered orally as tablets, aqueous or oily suspensions, lozenges, troches, powders, granules, emulsions, capsules, syrups or elixirs. The composition for oral use may contain one or more agents selected from the group of sweetening agents, flavoring agents, coloring agents and preserving agents in order to produce pharmaceutically elegant and palatable preparations. The tablets contain the acting ingredient in admixture with non-toxic pharmaceutically acceptable excipients that are suitable for the manufacture of tablets. These excipients may be, for example, (1) inert diluents, such as calcium carbonate, lactose, calcium phosphate, carboxymethylcellulose, or sodium phosphate; (2) granulating and disintegrating agents, such as corn starch or alginic acid; (3) binding agents, such as starch, gelatin or acacia; and (4) lubricating agents, such as magnesium stearate, stearic acid or talc. These tablets may be uncoated or coated by known techniques to delay disintegration and absorption in the gastrointestinal tract and thereby provide a sustained action over a longer period. For example, a time delay material such as glyceryl monostearate or glyceryl distearate may be employed. Coating may also be performed using techniques described in the U.S. Pat. Nos. 4,256,108; 4,160,452; and 4,265,874 to form osmotic therapeutic tablets for control release.

According to the invention, pharmaceutical compositions can be prepared for oral administration of therapeutic treatment for a cell-proliferative disorder that take advantage of the toxicity protection afforded by the compounds of Formula 1, comprising (a) paclitaxel or docetaxel in an amount exceeding about 550 milligrams, in the form of a free compound or its pharmaceutically-acceptable pro-drug, metabolite, analogue, derivative or salt, and (b) a toxicity-protecting amount of a compound of Formula 1 in the form of a free compound or its pharmaceutically-acceptable pro-drug, metabolite, analogue, derivative or salt. In preferred embodiments, at least about 650 milligrams of paclitaxel or docetaxel are employed. In further preferred embodiments, at least about 775 milligrams of paclitaxel or docetaxel are employed.

The compound of Formula 1, in the form of a free compound or a pharmaceutically-acceptable pro-drug, metabolite, analogue, derivative, solvate or salt , as well as the active pharmaceutical and chemotherapeutic agents useful in the methods of the invention can be administered, for in vivo application, parenterally by injection or by gradual perfusion over time independently or together. Administration may be intravenously, intraperitoneally, intramuscularly, subcutaneously, intracavity, or transdermally. For in vitro studies the agents may be added or dissolved in an appropriate biologically acceptable buffer and added to a cell or tissue.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, growth factors and inert gases and the like.

The invention can be broadly used to treat diseases, including but not limited to cancers, of the following: (a) an organ, including breast, lung, prostate, kidney, ovary, uterus, liver, pancreas, adrenal gland, and (b) a system, including the epithelial, gastric, intestinal, exocrine, endocrine, lymphatic, hematopoietic, genitourinary, colorectal, or central nervous system, and (c) head, neck or skin tissue.

Therefore, the present invention encompasses methods for ameliorating diseases, including but not limited to disorders associated with cell proliferation, neoplasms, cancers and the like, including treating a subject having the disorder, at the site of the disorder, with a compound of Formula 1, in the form of a free compound or a pharmaceutically-acceptable pro-drug, metabolite, analogue, derivative, solvate or salt, and a chemotherapeutic or pharmaceutical agent in an amount sufficient to inhibit or ameliorate the cell's proliferation or the disorder. Generally, the terms “treating”, “treatment” and the like are used herein to mean affecting a subject, tissue or cell to obtain a desired pharmacologic and/or physiologic effect. The effect may be prophylactic in terms of completely or partially preventing a disease or cell proliferative disorder or sign or symptom thereof, and/or may be therapeutic in terms of a partial or complete cure for a disorder and/or adverse effect attributable to, for example, aberrant cell proliferation. “Treating” as used herein covers any treatment of, or prevention of a disease or cell proliferative disorder in a vertebrate, a mammal, particularly a human, and includes: (a) preventing the disease or disorder from occurring in a subject that may be predisposed to the disease or disorder, but has not yet been diagnosed as having it; (b) inhibiting the disease or disorder, i.e., arresting its development; or (c) relieving or ameliorating the disease or disorder, i.e., cause regression of the disease or disorder.

The invention includes various pharmaceutical compositions useful for ameliorating diseases and cell proliferative disorder, including neoplasms, cancers and the like. The pharmaceutical compositions according to one embodiment of the invention are prepared by bringing a compound of Formula 1, in the form of a free compound or a pharmaceutically-acceptable pro-drug, metabolite, analogue, derivative, solvate or salt, and one or more pharmaceutical or chemotherapeutic agents or combinations of the compound of Formula 1 and one or more pharmaceutical or chemotherapeutic agents into a form suitable for administration to a subject using carriers, excipients and additives or auxiliaries. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol and polyhydric alcohols. Intravenous vehicles include fluid and nutrient replenishers. Preservatives include antimicrobial, anti-oxidants, chelating agents and inert gases. Other pharmaceutically acceptable carriers include aqueous solutions, non-toxic excipients, including salts, preservatives, buffers and the like, as described, for instance, in Remington's Pharmaceutical Sciences, 15th ed. Easton: Mack Publishing Co., 1405-1412, 1461-1487 (1975) and The National Formulary XIV., 14th ed. Washington: American Pharmaceutical Association (1975), the contents of which are hereby incorporated by reference. The pH and exact concentration of the various components of the pharmaceutical composition are adjusted according to routine skills in the art. See Goodman and Gilman's The Pharmacological Basis for Therapeutics (7th ed.).

The pharmaceutical compositions are preferably prepared and administered in dose units. Solid dose units are tablets, capsules and suppositories. For treatment of a subject, depending on activity of the compound, manner of administration, nature and severity of the disorder, age and body weight of the subject, different daily doses can be used. Under certain circumstances, however, higher or lower daily doses may be appropriate. The administration of the daily dose can be carried out both by single administration in the form of an individual dose unit or else several smaller dose units and also by multiple administration of subdivided doses at specific intervals.

The pharmaceutical compositions according to the invention may be administered locally or systemically in a therapeutically effective dose. Amounts effective for this use will, of course, depend on the severity of the disease and the weight and general state of the subject. Typically, dosages used in vitro may provide useful guidance in the amounts useful for in situ administration of the pharmaceutical composition, and animal models may be used to determine effective dosages for treatment of particular disorders. Various considerations are described, e.g., in Langer, Science, 249:1527, (1990); Gilman et al. (eds.) (1990), each of which is herein incorporated by reference. Dosages for parenteral administration of active pharmaceutical agents can be converted into corresponding dosages for oral administration by multiplying parenteral dosages by appropriate conversion factors. As to general applications, the parenteral dosage in mg/m² times 1.8=the corresponding oral dosage in milligrams (“mg”). As to oncology applications, the parenteral dosage in mg/m² times 1.6=the corresponding oral dosage in mg. See the Miller-Keane Encyclopedia & Dictionary of Medicine, Nursing & Allied Health, 5^{th} Ed., (W. B. Saunders Co. 1992). pp. 1708 and 1651.

The method by which the compound of Formula 1 may be administered for oral use would be, for example, in a hard gelatin capsule wherein the active ingredient is mixed with an inert solid diluent, or soft gelatin capsule, wherein the active ingredient is mixed with a co-solvent mixture, such as PEG 400 containing Tween-20. A compound of Formula 1 may also be administered in the form of a sterile injectable aqueous or oleaginous solution or suspension. The compound of Formula 1 can generally be administered intravenously or as an oral dose of 0.5 to 10 mg/kg given every 12 hours, 1 to 3 times before and 1 to 3 times after the administration of the pharmaceutical or chemotherapeutic agent, with at least one dose 1 to 4 hours before and at least one dose within 8 to 12 hours after the administration of the chemotherapeutic agent.

Formulations for oral use may be in the form of hard gelatin capsules wherein the active ingredient is mixed with an inert solid diluent, for example, calcium carbonate, calcium phosphate or kaolin. They may also be in the form of soft gelatin capsules wherein the active ingredient is mixed with water or an oil medium, such as peanut oil, liquid paraffin or olive oil.

Aqueous suspensions normally contain the active materials in admixture with excipients suitable for the manufacture of aqueous suspension. Such excipients may be (1) suspending agent such as sodium carboxymethyl cellulose, methyl cellulose, hydroxypropylmethylcellulose, sodium alginate, polyvinylpyrrolidone, gum tragacanth and gum acacia; (2) dispersing or wetting agents which may be (a) naturally occurring phosphatide such as lecithin; (b) a condensation product of an alkylene oxide with a fatty acid, for example, polyoxyethylene stearate; (c) a condensation product of ethylene oxide with a long chain aliphatic alcohol, for example, heptadecaethylenoxycetanol; (d) a condensation product of ethylene oxide with a partial ester derived from a fatty acid and hexitol such as polyoxyethylene sorbitol monooleate, or (e) a condensation product of ethylene oxide with a partial ester derived from fatty acids and hexitol anhydrides, for example polyoxyethylene sorbitan monooleate.

The pharmaceutical compositions may be in the form of a sterile injectable aqueous or oleagenous suspension. This suspension may be formulated according to known methods using those suitable dispersing or wetting agents and suspending agents that have been mentioned above. The sterile injectable preparation may also a sterile injectable solution or suspension in a non-toxic parenterally-acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

A compound of Formula 1 may also be administered in the form of suppositories for rectal administration of the drug. These compositions can be prepared by mixing the drug with a suitable non-irritating excipient that is solid at ordinary temperature but liquid at the rectal temperature and will therefore melt in the rectum to release the drug. Such materials include cocoa butter and polyethylene glycols.

The compounds of Formula 1 as used in the present invention may also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

For topical use, creams, ointments, jellies, solutions or suspensions, etc., containing the compounds of Formula 1 are employed.

Dosage levels of the compounds of Formula 1 as used in the present invention are of the order of about 0.5 mg to about 20 mg per kilogram body weight, an average adult weighing 70 killograms, with a preferred dosage range between about 5 mg to about 20 mg per kilogram body weight per day (from about 0.3 gms to about 1.2 gms per patient per day). The amount of the compound of Formula 1 that may be combined with the carrier materials to produce a single dosage will vary depending upon the host treated and the particular mode of administration. For example, a formulation intended for oral administration to humans may contain about 5 mg to 1 g of a compound of Formula 1 with an appropriate and convenient amount of carrier material that may vary from about 5 to 95 percent of the total composition. Dosage unit forms will generally contain between from about 5 mg to 500 mg of Formula 1 active ingredient.

It will be understood, however, that the specific dose level for any particular patient will depend upon a variety of factors including the activity of the specific compound employed, the age, body weight, general health, sex, diet, time of administration, route of administration, rate of excretion, drug combination and the severity of the particular disease undergoing therapy.

In addition, some of the compounds of the instant invention may form solvates with water or common organic solvents. Such solvates are encompassed within the scope of the invention.

In additional embodiments, the compounds of Formula 1 as free compounds, or in the form of pharmaceutically-acceptable pro-drugs, metabolites, analogues, derivatives, solvates or salts, can be used to significantly enhance the bioavailability of orally administered pharmaceuticals or chemotherapeutic agents that (i) bind to or are substrates for P-gp, and/or (ii) are taxane analogues, as identified above. The compounds of Formula 1 are broadly applicable to increase the bioavailability of co-administered active pharmaceutical agents that are within these classes of compounds. In addition, the compounds of Formula 1 further provide protection of the subject mammal against the inherent toxicity of such active pharmaceutical agents, facilitating delivery to the target cells of higher dosages of the active pharmaceutical agents under conditions of reduced toxicity.

In a preferred embodiment, the present invention provides compositions and methods whereby chemotherapeutic agents as identified above, such as, for example, taxanes (e.g., paclitaxel), vinca alkaloids, anthracyclines, and epidophyllotoxins could be administered orally in a therapeutically effective manner. The studies provided herein (e.g., see Examples) indicate that co-administration of a Formula 1 compound with a chemotherapeutic agent, such as paclitaxel, enhances the oral bioavailability of such agents. Oral co-administration of agents such as, for example, paclitaxel, in an appropriate vehicle, with a compound of Formula 1 results in therapeutically useful blood levels of the chemotherapeutic agent. The method by which the cytotoxic chemotherapeutic agent may be administered for oral use would be, for example, in solution as a microemulsion, in a hard gelatin capsule wherein the active ingredient is mixed with an inert solid diluent, in a soft gelatin capsule wherein the active ingredient is dissolved in a co-solvent mixture such as PEG 400 containing Tween-20 and ethanol, or as a solid dispersion contained in a hard gelatin capsule. Thus, for example, paclitaxel can be given orally at a dose of 2 to 20 mg/kg once every week, once every four days, or once every three weeks, with co-administration of the compound of Formula 1 at an oral dose of 2 to 10 mg/kg, formulated in a hard gelatin capsule wherein the active ingredient is mixed with an inert solid diluent, or in a soft gelatin capsule, wherein the active ingredient is dissolved in a co-solvent mixture, such as PEG 400 containing Tween-20.

In further embodiments, the compounds of Formula 1 as free compounds, or in the form of pharmaceutically-acceptable pro-drugs, metabolites, analogues, derivatives, solvates or salts, can be used to deliver orally or parenterally administered pharmaceuticals or chemotherapeutic agents that (i) bind to or are substrates for P-gp, and/or (ii) are taxane analogues, as identified above, across the blood-brain barrier for therapeutic or preventative purposes. P-gp is naturally expressed in the blood-brain barrier where it serves to prevent systemic toxins and xenobiotics from entering the brain. In some cases, such as in the treatment of neurological disorders or HIV/AIDS, it is desirable to have a therapeutic agent traverse the blood-brain barrier and exert its effect in the brain. Numerous anti-seizure medicines such as phenytoin, opiates such as loperamide and morphine, and HIV protease inhibitors such as saquinavir and nelfinavir are all P-gp substrates that are subject to active efflux across the blood-brain barrier. The compounds of Formula 1 are broadly applicable to facilitating penetration across the blood-brain barrier by co-administered active pharmaceutical agents that are within these classes of compounds. In addition, the compounds of Formula 1 further provide protection of the subject mammal against the inherent toxicity of such active pharmaceutical agents, facilitating delivery to the target cells of high dosages of the active pharmaceutical agents under conditions of reduced toxicity.

The invention is particularly useful for the administration of protease inhibitors in human immunodeficiency virus therapy. In these embodiments, the active pharmaceutical agent is a protease inhibitor in the form of a free compound or its pharmaceutically-acceptable pro-drug, metabolite, analogue, derivative, solvate or salt. In one embodiment the protease inhibitor is saquinavir. If parenteral administration of saquinavir is desired, then the toxicity-protected dosage range during a given treatment session is about 13 mg/kg to about 39 mg/kg. In preferred practice for such parenteral administration, the toxicity-protected dosage is about 19 mg/kg to about 39 mg/kg. In the case of oral administration of saquinavir, the toxicity-protected dosage is about 600 mg to about 2400 mg per treatment session; preferably about 1200 mg to about 2400 mg per treatment session.

In further embodiments the invention provides compositions comprising a compound of Formula 1 in the form of pharmaceutically-acceptable pro-drugs, metabolites, analogues, derivatives, solvates or salts in admixture with an active pharmaceutical agent or chemotherapeutic agent, together with a pharmaceutically acceptable diluent, adjuvant, or carrier.

The invention will now be described in greater detail by reference to the following non-limiting examples.

### EXAMPLES

MDR cell lines are easily obtainable for in vitro determination of drug sensitization and treatment by compounds of the present invention. In vitro potentiation of antineoplastic cytotoxicity by the imidazole derivatives of the present invention can be measured, for example, in both CEM/VLB1000 and SK/VLB1000 cell lines. These multidrug resistant cell lines can be obtained from Dr. Victor Ling, Ontario Cancer Institute, Toronto, Canada. The CEM/VLB 1000 cell line was maintained as a suspension in minimum essential medium supplemented with 10% fetal bovine serum in a humidified atmosphere of 95% air and 5% CO₂ while the SK/VLB 1000 cell line was maintained as adherent cells using the identical medium conditions as for the CEM cells. The CEM/VLB 1000 cells are typically seeded at a density of 5×10⁴ cells/well in a 96 well microtiter plate while the SK/VLB 1000 cell line is typically seeded at a density of 2,500 cells/well after trypsinization. Vinblastine (5 μM, for the CEM cells) or Taxol (3 μM, for the SK cells) and the compound of Formula 1 (0.01 to 50 μM) can be added directly to the wells. After an incubation of 72 hours in presence of drug, Alamar Blue (B. Page et al., Int. J. Oncol. 3:473-476, 1993) is added (10 μL to the 200 μL cell suspension) for a period of 4-6 hours after which the fluorescence (excitation=530 nM, emission=590 nM) is read for each well using a “CytoFluor” microtiter fluorometer plate reader. This assay measures the effective concentration of compound necessary to enhance the cytotoxicity (EC₅₀) of vinblastine or taxol in the MDR cell line.

In Vivo Antitumor Efficacy Models. Anti-tumor efficacy experiments with orthotopic MDA/LCC6 and MDA/LCC6^{MDR1} tumors were conducted in female SCID/RAG2 mice. Ascites propagated cells (2×10⁶ in 50 μl) were injected into mammary fat pads bilaterally on day 0 before randomization into groups of 5 mice per group. Paclitaxel (12 mg/kg/dose) was administered QD i.v. (tail vein) in 200 μl saline/Cremophor/ethanol (8/1/1 by volume) on days 5, 12, 19, and 26. The compound of Formula 2-free base (30 mg/kg/dose) was administered BID p.o. by gavage in 100 μl PEG 400/Tween 20 (9/1 by volume) on days 4-6, 11-13, 18-20, and 25-27. Mean body weights were recorded at least every other day. Tumor weight was monitored approximately every other day by caliper measurements and calculated according to the formula (Tumor weight=(length×width²)÷2). This conversion formula was verified by comparing the calculation derived tumor weights to excised and weighed tumors. Animals bearing ulcerated tumors or where tumor weight exceeded 10% of the animal's body weight were terminated. The weights of the bilateral tumors were averaged for each mouse and mean tumor weights for each treatment group±standard error of the mean were calculated. Statistical analysis was carried out by Mann-Whitney test using GraphPad Prism software (San Diego, Calif.).

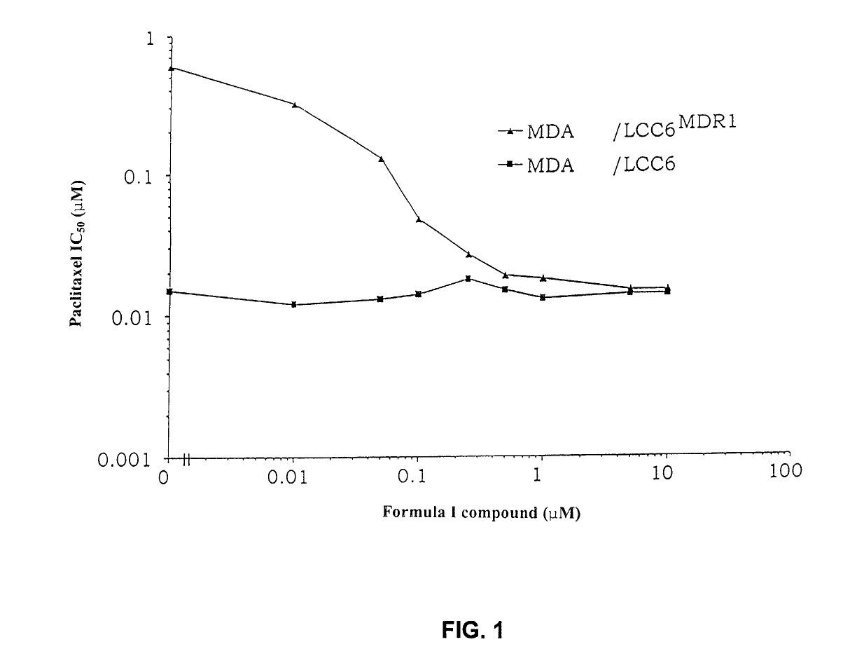

The compound of Formula 2 was administered the afternoon before, 2 hours before and 6 hours after each paclitaxel dose. The ability of orally administered Formula 2 compound to reverse MDR in solid tumors in vivo was assessed with the MDA/LCC6^{MDR1} model. Because the compound of Formula 2 produced no effect on paclitaxel blood levels, focus on up-front therapy (for naive animals) or minimal residual disease models was maintained, as opposed to established tumors. MDA/LCC6^{MDR1} cells that express P-gp were resistant to paclitaxel treatment in vitro and this resistance was reversed by the compound of Formula 2 (FIG. 1). Treatment with paclitaxel or the compound of Formula 2 alone had no significant effect on in vivo tumor growth compared to the vehicle control (see, for example, Newman et al., Ca. Res. 60:2964, 2000, which is incorporated by herein by reference in its entirety). When the Formula 2 compound and paclitaxel were combined, there was a statistically significant inhibition of tumor growth that persisted for at least two weeks after the last dose of paclitaxel. The growth delay produced by paclitaxel and the Formula 2 compound in the MDA/LCC6^{MDR1} xenografts was comparable to the growth delay produced by paclitaxel alone in MDA/LCC6 xenografts. Similar results were obtained in three independent experiments with two different strains of immunocompromised mice (SCID/RAG-2 and athymic). Similar results were obtained with non-established and established tumors.

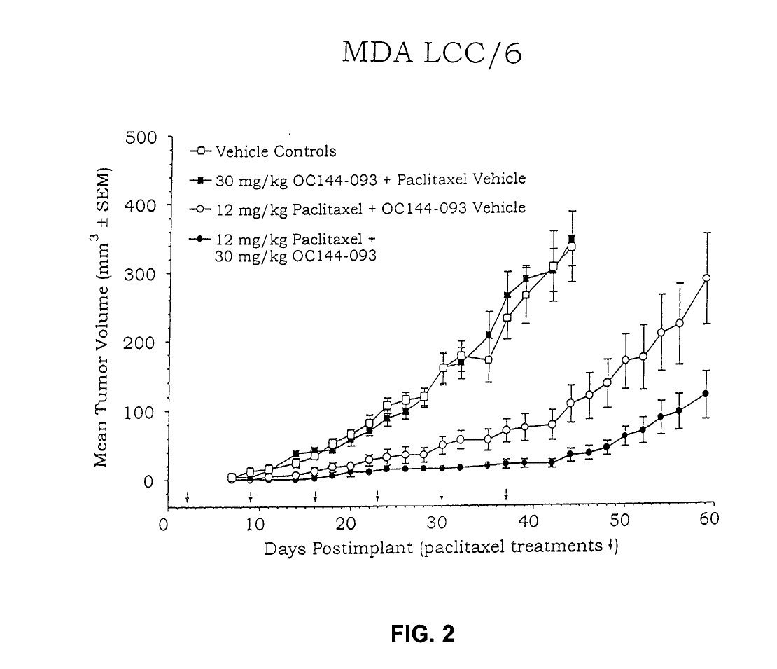

The compound of Formula 2 did not enhance the in vitro anti-tumor activity of paclitaxel against breast carcinoma cells that do not express P-gp (FIG. 1). Surprisingly, the compound of Formula 2 did enhance the in vivo anti-tumor activity of paclitaxel against the same non-P-gp expressing tumor cells (FIG. 2).

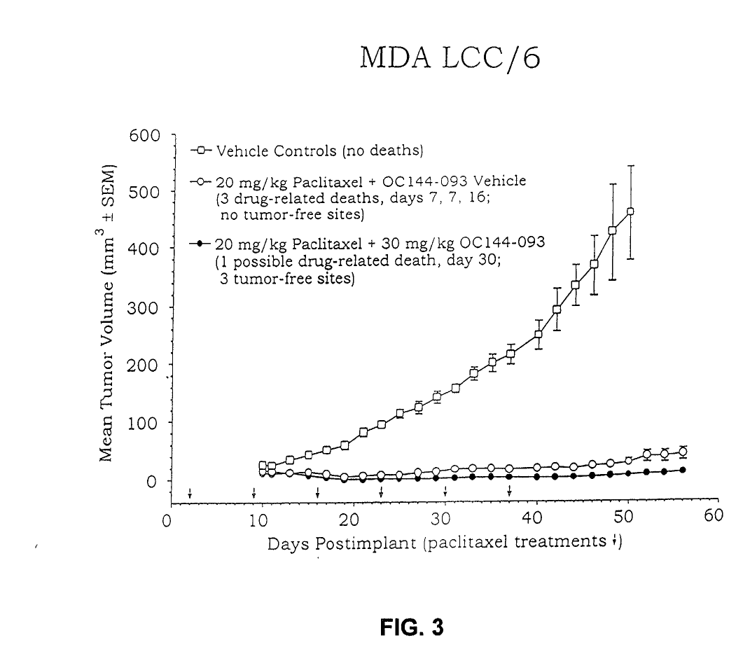

Non-tumor bearing athymic mice were treated with the compound of Formula 2 and (or) paclitaxel as described. The compound of Formula 2 decreased paclitaxel toxicity in the athymic mice independent of tumor presence (see Table 1). The ability of the compound of Formula 2 to protect mice from lethal doses of paclitaxel allowed administration to tumor-bearing mice at doses higher than standard levels (FIG. 3). Administration of 20 mg/kg paclitaxel alone once per week resulted in 50% lethality, with 3 of 6 animals dying within 16 days. This high LD₅₀ dose of paclitaxel significantly inhibited tumor growth, but did not completely prevent tumor formation at any injection site. Administration of 20 mg/kg paclitaxel with the compound of Formula 2 reduced lethality to one possible drug-related death and completely prevented tumor formation at several injection sites (FIG. 3).

TABLE 1
Effect of Paclitaxel Toxicity in Athymic Mice in the Presence and
Absence of Formula 2 Compound

| Treatment | Survival |
|---|---|
| Vehicle + Formula 2 compound | 4/4 |
| Paclitaxel + Vehicle | 0/4 |
| Paclitaxel + Formula 2 compound | 4/4 |

Paclitaxel: 24 mg/kg i.v. Q5D × 6 + 36 mg/kg i.v. Q5D × 2
Compound of Formula 2:30 mg/kg p.o. afternoon before and BID day of paclitaxel treatments

Modulation of P-gp-Mediated Drug Resistance in Vitro. The compound of Formula 2 was able to reverse resistance to all classes of P-gp substrates in a wide variety of tumor cell types with EC₅₀s in the low nM range (Table 2). Similar results were obtained with etoposide in several models. Complete reversal of MDR was typically seen with Formula 2 doses between 0.25 and 1.0 μM. Examples with cells expressing extremely high levels of P-gp as a result of drug selection (CEM/VLB1000), moderate levels of P-gp from gene transduction (MDA/LCC6^{MDR1}), and low (intrinsic) levels of P-gp (HCT-15) are illustrated in Table 2. The compound of Formula 2 had no effect on doxorubicin or paclitaxel IC₅₀s in non-P-gp-expressing CCRF-CEM and MDA/LCC6 cells, respectively (FIG. 1, for MDA/LCC6 cells). The Formula 2 compound retained full MDR reversal potency after incubation in human plasma, suggesting that protein binding mediated-inactivation will not be a problem in humans.

TABLE 2
Reversal of MDR by the Compound of Formula 2 in P-gp-expressing
Cell Lines
The IC₅₀s for the indicated anti-tumor agents were determined
in the presence of various concentrations of the Formula 2 compound
as described. The Formula 2 compound EC₅₀ is the concentration
that produced half maximal reversal of anti-tumor agent resistance. Each
experiment was carried out two to four times.

| | Formula 2 compound EC₅₀ (μM ± SD) | | |
|---|---|---|---|
| Cell Line | Doxorubicin | Vinblastine | Paclitaxel |
| CEM/VLB1000^{a} | 0.09 ± 0.06 | 0.07 ± 0.01 | |
| MES-SA/DX5^{b} | 0.024 ± 0.006 | 0.034 ± 0.007 | 0.027 ± 0.007 |
| SK/VLB1000^{c} | 0.025 ± 0.008 | 0.015 | 0.033 |
| MCF-7/ADR^{d} | 0.038 ± 0.006 | 0.02 ± 0.01 | 0.031 ± 0.004 |
| MDA/LCC6^{MDR1d} | 0.013 | | 0.009 |
| HCT-15^{e} | | | .0016 |

^{a}lymphoma,
^{b}uterine,
^{c}ovarian,
^{d}breast,
^{e}colorectal carcinoma

Non-Specific Toxicity of the Compound of Formula 2/Cell Proliferation Assays. Cell proliferation IC₅₀s and MDR reversal EC₅₀s were determined from 3-day dose-response curves carried out in triplicate in 96-well plates essentially as described by Monks et al. (J. Natl. Cancer Inst., 83:757-66, 1991). Cells were plated in standard growth medium at 2.5 or 5.0×10⁴ per well (CCRF-CEM and CEM/VLB1000 respectively) or 5×10³ per well (all other cell lines) in a final volume of 100 μl. After a 2 h incubation for non-adherent cells and overnight incubation for adherent cells, the initial cell density was determined by fluorescence readout of Alamar Blue metabolism. The compound of Formula 2, cytotoxic agents, or compound vehicles were added to duplicate plates and the incubation was continued for an additional 72 h. Final cell density was determined with Alamar Blue. In some experiments, a standard endpoint assay was used without analysis of initial cell density with Alamar Blue. Similar results were obtained with the two assays. EC₅₀s were derived by nonlinear regression analysis assuming a sigmoidal dose-response using GraphPad Prism Software (San Diego, Calif.). Proliferation assays capable of measuring both cytostatic and cytotoxic responses were carried out as described herein with 15 non-transformed and transformed cell lines, including primary fibroblasts, non-transformed smooth muscle, leukemia, breast, colon, ovarian and uterine carcinoma cells (±P-gp expression). Although the compound of Formula 2 reversed P-gp-mediated MDR in the low nanomolar concentration range, the compound was non-cytotoxic by itself at doses up to 100 μM in all cell lines. Cytostatic IC₅₀s ranged from 6 to 170 μM, with an average value of 60 μM (Table 3). IC₅₀s for non-specific cytotoxicity were similar in matched cell lines plus and minus P-gp expression. The results indicate that the compound of Formula 2 is probably not a P-gp transport substrate. If it were, some consistent degree of resistance would be expected in P-gp expressing cells.

TABLE 3
Effect of the Compound of Formula 2 on the Proliferation of
Various Cell Lines
The IC₅₀ for inhibition of the growth of cell lines by the
compound of Formula 2 was determined as described. Each experiment
was carried out at least two times with similar results.

| | P-gp^{a} | |
|---|---|---|
| Human Cell Type/Line | Expression | Formula 2 compound IC₅₀ |
| Primary fibroblast | | |
| CCD-986SK | | >100 |
| Smooth muscle | | |
| HISM | | >100 |
| Lymphoma | | |
| CEM | Neg | 38 |
| CEM/VLB1000 | Pos | 32 |
| Ovarian carcinoma | | |
| SKOV3 | Neg | 48 |
| SK/VLB1000 | Pos | 15 |
| Uterine carcinoma | | |
| MES-SA | Neg | 51 |
| Breast carcinoma | | |
| MCF-7 | Neg | 30 |
| MCF-7/ADR | Pos | 68 |
| MDA/LCC6 | Neg | >100 |
| MDA/LCC6^{MDR1} | Pos | >100 |
| Colorectal carcinoma | | |
| SW480 | Pos | >100 |
| SW620 | Neg | >100 |
| HT-29 | Neg | 12 |
| HCT-15 | Pos | 6 |

^{a}Direct analysis and (or) from literature.

Pharmacokinetic Studies. Oral Paclitaxel Bioavailability Enhancement. A total of 21 male Sprague Dawley rats were used. Animals were approximately 8 weeks of age at arrival and weighed ca 327-359 g. Four males were randomly assigned to each of Groups 1 and 2. On the day of dosing and following an overnight fast, animals were weighed (ca 308-326 g) and administered their respective combination of doses. Animals of Group 1 received an oral dose of the compound of Formula 2 mesylate salt (10 mg of the free base/kg) by gavage at a dose volume of 1 mL/kg. Animals of Group 2 received the mesylate salt vehicle (deionized water) by gavage at a dose volume of 1 ml/kg. Sixty minute later, all animals in Groups 1 and 2 received a dose of Paclitaxel (40 mg/kg) by gavage at a dose volume of 2 ml/kg and a concomitant dose of either the Formula 2 compound or vehicle.

Following administration of paclitaxel, blood samples (ca 0.3 ml in EDTA were obtained from all rats by jugular venipuncture during anesthesia with isolurane (Abbott Laboratories) at each of the following time points: pre-dose (prior to administration of the Formula 2 compound or vehicles) and at 0.25, 0.5, 1, 2, 4, 6, and 8 hours following administration of the paclitaxel dose. Immediately following collection, all samples were placed on ice until further processing or storage. Blood samples were centrifuged (ca 3200 g at about 4° C. for 10 minutes) and the resulting plasma samples were stored at −20° C. pending analysis for unchanged paclitaxel. Following analysis of paclitaxel, all remaining plasma were tested for a determination of the Formula 2 compound concentration.

Plasma samples were analyzed for unchanged paclitaxel using a liquid chromatography/mass spectroscopy (“LC/MS”) assay. Pharmacokinetic analysis was performed on plasma concentrations using the PhAST Software Program, Version 2.2-00 (Phoenix International Life Sciences, Inc.). The highest observable concentration was used as the peak concentration (Cₘₐₓ). Time to Cₘₐₓ was denoted as Tₘₐₓ. The area under the plasma concentration vs. time-curve from time zero to the last measurable concentration (AUC(0-t)) was calculated by the linear trapezoidal method. The area under the plasma concentration curve extrapolated to infinity (AUC(l)) was calculated as the sum of AUC(0-t) plus the ratio of the last plasma concentration to the elimination rate constant. The terminal elimination constant (K) was calculated from the last 3 non-zero points of the log-linear regression. Half-life (t^{½}) was determined by dividing 0.693 by K. The apparent plasma clearance (CL/F) was calculated for paclitaxel by dividing the nominal dose administered by AUC(l). Where applicable, numerical values were subjected to calculation of group means and % CV. Where values were below the limit of quantification, zero was used for pharmacokinetic analysis. Statistical analysis consisted of a Student-T-test with Two-sample unequal variance approximation and was performed on AUC90-t) and Cₘₐₓ values to compare the Formula 2 compound treated animals vs. the corresponding vehicle treated animals.

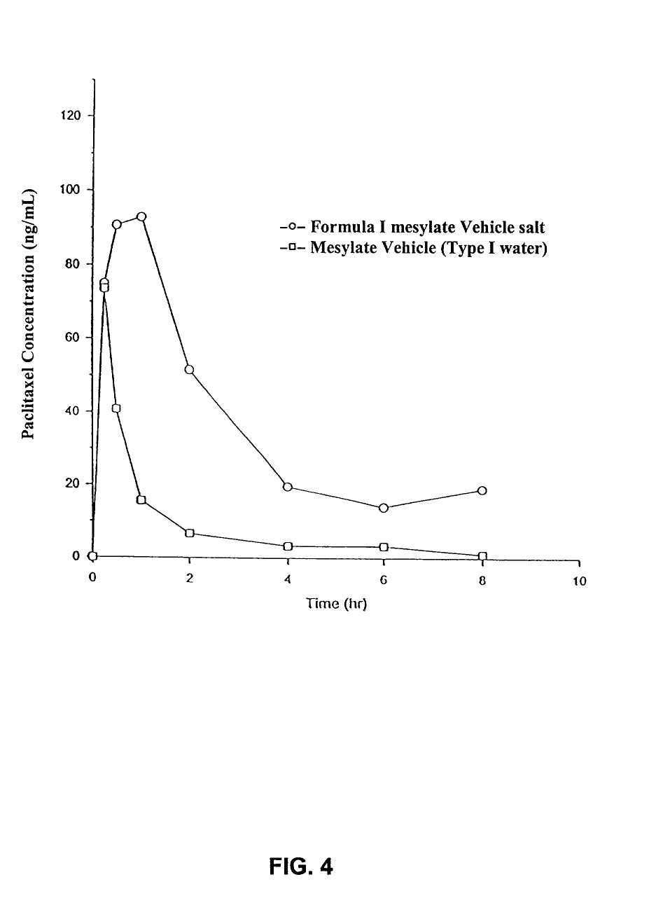

Linear graphical representations of the concentration of paclitaxel in plasma vs. time, and paclitaxel/mesylate salt vs. time, are presented in FIG. 4.

Mean terminal phase half-life (t^{½}) values for paclitaxel were calculated to be 3.5 and 3.9 hr for Groups 1 and 2, respectively, suggesting a relatively moderate elimination of the parent drug following oral administration. The mean AUC(0-t) and AUC(l) values for paclitaxel in plasma was higher in both groups receiving the compound of Formula 2 sixty minutes prior to and concomitantly with the administration of paclitaxel. The mean AUC(0-t) values for Group 1 (mesylate salt) were 286 ng hr/ml. The values for the corresponding control group were 67 ng hr/ml (Group 2). The difference in AUC (0-t) between Group I and Group 2 was statistically different (P=0.003).

Mean Cₘₐₓ values were also higher for both Formula 2 treated groups when compared to their corresponding vehicles. These differences, however, were not statistically significant (P=0.22 for the mesylate salt form). These results suggest that the mesylate salt of Formula 2 significantly increased the oral bioavailability of paclitaxel. The AUC(0-t) value was over 4 times higher for the mesylate salt animals than for their vehicle treated counterpart.

Enhancement of the Oral Bioavailability of Paclitaxel and Docetaxel in Rats

In a further example, separate groups of eight male Sprague-Dawley rats (250-300 g body weight) with cannulae inserted into the jugular vein were purchased from Harlan-Sprague Dawley (Madison, Wis.). Animals were allowed free movement and access to water but were fasted for at least 8 h prior to the dose until study completion. Rats were orally administered paclitaxel (10 mg/kg) or docetaxel either alone or with (2-[4-(3-ethoxy-1-propenyl)phenyl]-4,5-bis(4-(2-propylamino)phenyl)-1H-imidazole free base (10 and 50 mg/kg) or (2-[4-(3-ethoxy-1-propenyl)phenyl]-4,5-bis(4-(2-propylamino)phenyl)-1H-imidazole mesylate salt (5 and 10 mg/kg). The paclitaxel formulation was prepared by dissolving paclitaxel in ethanol then mixing 1:1 with Cremophor EL. Docetaxel was dissolved in polysorbate 80, then diluted in 13% ethanol in sterile water. (2-[4-(3-ethoxy-1-propenyl)phenyl]-4,5-bis(4-(2-propylamino)phenyl)-1H-imidazole free base was dissolved in PEG 400 and mixed with the docetaxel or paclitaxel formulations. Immediately prior to administration the mixture was diluted in saline to provide a paclitaxel or docetaxel concentration of 1 mg/ml. Animals were then administered 10 μl/g body weight by oral gavage. (2-[4-(3-ethoxy-1-propenyl)phenyl]-4,5-bis(4-(2-propylamino)phenyl)-1H-imidazole mesylate was dissolved in sterile saline then mixed 1:1 with PEG 400. Immediately before dosing this formulation was diluted with sterile saline, combined with the paclitaxel or docetaxel formulations, then animals were administered 10 μl/g body weight by oral gavage.

Serial blood samples (500 μl) were drawn prior to each dose (time 0) and at 0.5, 1, 2, 3, 4, 6 and 8 h through a cannula inserted into the jugular vein. The blood was collected into Microtainer® tubes containing EDTA anticoagulant. Whole blood was centrifuged under refrigeration at 2800 rpm for 10-20 minutes. Plasma samples were stored at −20° C. until analysis using a validated HPLC-MS method. Maximum plasma paclitaxel concentration (C.,) and time to achieve this concentration (Tₘₐₓ) were measured from concentration vs. time profiles. Area under the concentration vs. time curve from 0-8 hours (AUC₀₋₈) was calculated using the linear and log trapezoidal methods in the rising and declining phases of the concentration vs. time curve respectively.

The results of the bioavailability-enhancement studies with docetaxel and paclitaxel are presented in Table 4. Co-administration of (2-[4-(3-ethoxy-1-propenyl)phenyl]-4,5-bis(4-(2-propylamino)phenyl)-1H-imidazole free base (10 mg/kg) caused a 4.3-fold increase in docetaxel Cₘₐₓ and a 7.4-fold increase in docetaxel AUC₀₋₈. The same dose of (2-[4-(3-ethoxy-1-propenyl)phenyl]-4,5-bis(4-(2-propylamino)phenyl)-1H-imidazole free base caused a 5.4-fold increase in paclitaxel Cₘₐₓ and a 6.9-fold increase in paclitaxel AUC₀₋₈. Increasing the (2-[4-(3-ethoxy-1-propenyl)phenyl]-4,5-bis(4-(2-propylamino)phenyl)-1H-imidazole dose to 50 mg/kg did not increase its effect on the bioavailability of either drug. When (2-[4-(3-ethoxy-1-propenyl)phenyl]-4,5-bis(4-(2-propylamino)phenyl)-1H-imidazole(10 mg/kg) was administered as the mesylate salt rather than the free base docetaxel Cₘₐₓ was increased 6.5-fold with and a 4.4-fold increase was observed for docetaxel AUC₀₋₈. Paclitaxel Cₘₐₓ and AUC₀₋₈ were increased 8.7-fold and 11.5-fold respectively by co-administration with (2-[4-(3-ethoxy-1-propenyl)phenyl]-4,5-bis(4-(2-propylamino)phenyl)-1H-imidazole mesylate (10 mg/kg).

TABLE 4
The effect of (2-[4-(3-ethoxy-1-propenyl)phenyl]-4,5-bis(4-(2-propylamino)phenyl)-1H-
imidazole mesylate salt (OCM) and free base (OCFB) on the oral bioavailability of the
chemotherapeutic drugs docetaxel and paclitaxel in rats.

| | Docetaxel | | Paclitaxel | |
|---|---|---|---|---|
| Doses | Cₘₐₓ (ng/ml) | AUC₀₋₈ (ng*h/ml) | Cₘₐₓ (ng/ml) | AUC₀₋₈ (ng*h/ml) |
| Drug alone (10 mg/kg) | 37 (28) | 32 (11) | 99 (50) | 266 (183) |
| Drug + 10 mg/kg OCFB | 159 (91) | 234 (146) | 531 (199) | 1823 (695) |
| Drug + 50 mg/kg OCFB | 175 (42) | 238 (108) | 241 (89) | 819 (228) |
| Drug alone (10 mg/kg) | 30 (13) | 69 (27) | 75 (42) | 189 (88) |
| Drug + 5 mg/kg OCM* | 217 (128) | 356 (181) | 437 (86) | 1498 (245) |
| Drug + 10 mg/kg OCM* | 199 (111) | 304 (159) | 653 (201) | 2181 (756) |

*Dose of the mesylate was corrected to provide 5 mg/kg or 10 mg/kg of the free base.

These data clearly illustrate the utility of (2-[4-(3-ethoxy-1-propenyl)phenyl]-4,5-bis(4-(2-propylamino)phenyl)-1H-imidazole as an enhancer of oral bioavailability. Although the present examples describe preclinical work with taxanes, similar results are anticipated with a broad range of cancer chemotherapeutic agents, including, but not limited to, paclitaxel, docetaxel, vinblastine, vincristine, vinorelbine, doxorubicin, daunorubicin, etoposide, topotecan, dactinomycin, plicamycin (mithramycin), mitomycin, verapamil, cytosine arabinoside (cytarabine), methotrexate, and irinotecan (CPT-11). Co-administration of (2-[4-(3-ethoxy-1-propenyl)phenyl]-4,5-bis(4-(2-propylamino)phenyl)-1H-imidazole may also improve the bioavailability of other classes of therapeutic agents including but not limited to taxanes, epothilones, discodermolide, eleutherobin, sarcodictyins, laulimalides, vinca alkaloids, anthracyclines, camptothecins, epipodophyllotoxins, methotrexate, angiotensin converting enzyme (ACE) inhibitors, human immunodeficiency virus protease inhibitors, antibiotics, calcium channel antagonists, β-blockers, HMG-CoA reductase inhibitors, immunosuppressive agents, opiates, fluoroquinolones, macrolide antibiotics, aminoglycoside antibiotics, antihistamines, anti-epileptic agents, anti-malarial agents, and dopamine agonists.

PK Interaction Studies. Plasma paclitaxel levels were determined in SCID/RAG2 mice following pre-treatment with 3 p.o. gavage doses of 30 mg/kg Formula 2 compound (free base) or vehicle (100 μl PEG 400/Tween 20; 9/1 by volume) 25, 19 and 1 h prior to a single i.v. dose of 16 mg/kg paclitaxel in 200 μl saline/Cremophor/ethanol (8/1/1 by volume). At the indicated times after paclitaxel administration, 3 mice per time point were anesthetized with CO₂ and blood was removed by cardiac puncture into Microtainer tubes containing EDTA. Plasma samples generated by centrifugation were extracted with acetonitrile and the analysis for paclitaxel content was carried out by HPLC using a Waters 600E multisolvent delivery system, 717 plus autosampler and 996 photodiode array detector. Baccatin III (0.8 nmoles/200 μl plasma) was used as the internal standard (IS) and added to plasma samples during extraction with acetonitrile. Standard curve samples as well as quality control samples were also prepared from spiked control plasma in order to verify the accuracy of the HPLC analysis. Paclitaxel and the IS were resolved on a Nova-Pak C₁₈ column (4 μm, 150 mm×3.9 mm inside diameter; Waters, Milford, Mass.) with double distilled water (A), and acetonitrile (B), using the following gradient profile: t=0 min, 10% B; t=5 min, 10% B; t=30 min, 65% B; t=40 min, 65% B; t=45 min, 10% B; t=50 min, 10% B. The gradient was formed using a high pressure mixer and the flow rate was 1.0 ml min⁻¹. A Waters 996 Photo Diode Array Detector was used to scan at multiple wavelengths and chromatograms were processed for traces obtained at 230 nm.

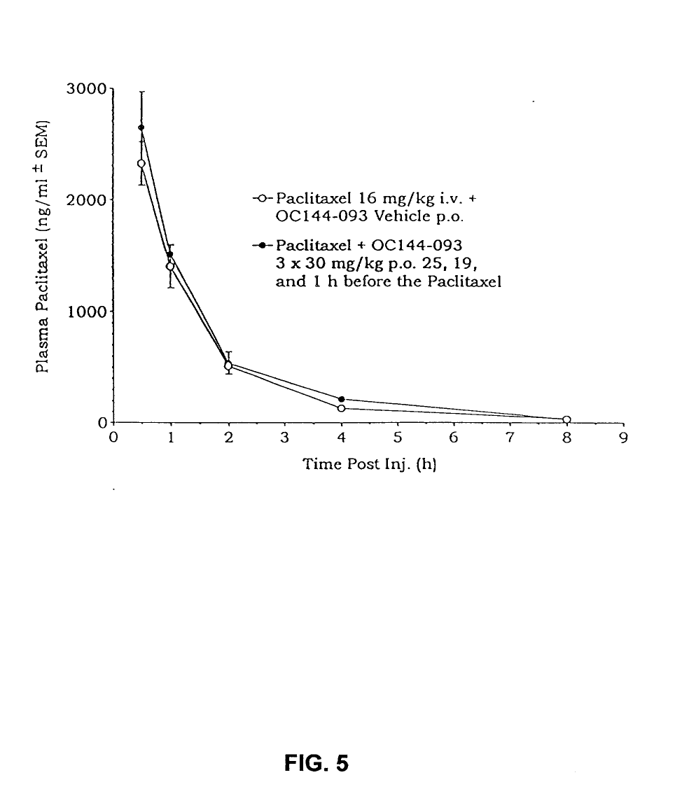

Two enzymes primarily responsible for metabolism of paclitaxel are P450 CYP3A4 and CYP2C8. Some P-gp inhibitors are also metabolized by P450 CYP 3A4, leading to inhibition of paclitaxel metabolism. This may contribute to PK interactions with paclitaxel. The Formula 2 compounds was not metabolized by P450 CYP3A4 or CYP2C8. The Kᵢ for the compound of Formula 2 inhibition of human CYP3A4-mediated paclitaxel metabolism was found to be 39.8±5.1 μM. This is approximately 1000-fold higher than the EC₅₀s for reversal of MDR by the Formula 2 compound, suggesting that the compound might not produce a significant PK interaction with paclitaxel in vivo. FIG. 5 demonstrates that pretreatment of mice with three oral doses of 30 mg/kg of the Formula 2 compound had no effect on i.v. plasma paclitaxel levels.

Enhancement of Blood-Brain Barrier Penetration of Loperamide in Mice

Mice were administered an intravenous bolus dose of loperamide (1, 2, 4, 8 or 16 mg/kg) either alone or with (2-[4-(3-ethoxy-1-propenyl)phenyl]-4,5-bis(4-(2-propylamino)phenyl)-1H-imidazole (OC144-093; also referred to as ONT-093; 10 mg/kg) then the animals were placed on a hot-plate at a moderately elevated temperature. The antinociceptive effect of loperamide (a reflection of drug transit across the blood brain barrier) was measured as an increase in the time taken by the mice to jump off the hot-plate.

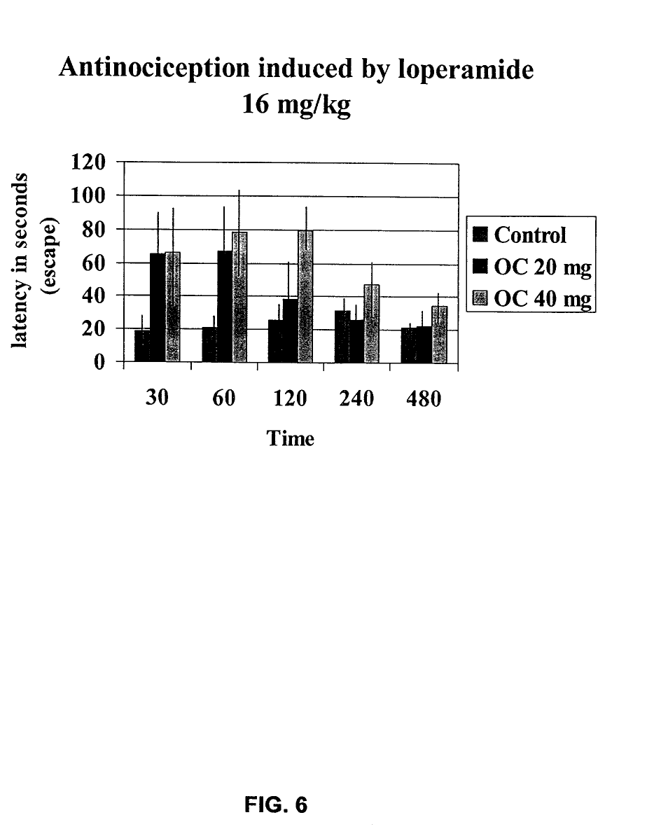

Data describing the effect of (2-[4-(3-ethoxy-1-propenyl)phenyl]-4,5-bis(4-(2-propylamino)phenyl)-1H-imidazole in mice are presented in FIG. 6, which shows the elapsed time (latency in seconds) until the mice escaped by jumping off the hot plate plotted versus elapsed time after administration of loperamide. In the absence of loperamide, the mean time to escape the hot-plate was 20 seconds either with or without (2-[4-(3-ethoxy-1-propenyl)phenyl]-4,5-bis(4-(2-propylamino)phenyl)-1H-imidazole. Loperamide alone had no effect on the mouse hot-plate escape time, however co-administration with either 20 mg or 40 mg of (2-[4-(3-ethoxy-1-propenyl)phenyl]-4,5-bis(4-(2-propylamino)phenyl)-1H-imidazole caused significant increases in the hot-plate escape time. These increases were dose dependent with respect to loperamide.

### Pharmacokinetics and Bioavailability of Saquinavir Following a Single Oral Dose of (2-[4-(3-ethoxy-1-propenyl)phenyl]-4,5-bis(4-(2-propylamino)phenyl)-1H-imidazole in Male Sprague Dawley Rats

The objective of this example was to obtain preliminary data on the effects of (2-[4-(3-ethoxy-1-propenyl)phenyl]-4,5-bis(4-(2-propylamino)phenyl)-1H-imidazole (referred to in this Example as ONT-093) on the bioavailability and pharmacokinetics of the protease inhibitor saquinavir following a single oral administration in male Sprague Dawley rats. Sixteen male Sprague Dawley rats exhibiting good general health were selected for this study. Each rat received either a single oral dose of ONT-093 (20 mg/kg) or a single oral dose of the vehicle followed 30 min later by a single oral dose of saquinavir (20 mg/kg). Blood samples (ca 0.3 mL) were collected from 8 selected rats by jugular venipuncture during slight anesthesia pre-dose and at 0.25, 0.5, 1, 2, 4 and 8 post-dose (saquinavir dose). Cerebrospinal fluid (CSF) and brain tissue were collected terminally from the remaining 8 rats at ca 30 min following administration of the dose of saquinavir. Blood samples were collected into tubes containing EDTA and placed on wet ice pending centrifugation. Plasma, CSF and brain tissue samples were stored at ca −80° C. until analysis of saquinavir by LC/MS. The vehicle used was polyethylene glycol (PEG-400)/Tween 20 (9: 1; v:v).

Oral Formulation of ONT-093

The formulation of ONT-093 for oral administration was prepared by dissolution of the appropriate masses of ONT-093 in a mixture of PEG-400/Tween 20 (9:1; v:v) to achieve a nominal concentration of 10 mg/mL. The formulation was slightly warmed up (to ca 40° C) in a water bath, vortexed and sonicated to ensure the complete dissolution of ONT-093. The formulation was stored at room temperature pending dose administration.

Oral Formulation of Saquinavir

The formulation of saquinavir was prepared by dissolution of the appropriate masses of saquinavir in a mixture of PEG-400/Tween 20 (9:1; v:v) to achieve a nominal concentration of 20 mg/mL. The formulation was slightly warmed up (to ca 40° C.) in a water bath, vortexed and sonicated to help and ensure the complete dissolution of saquinavir. The formulation was stored at room temperature pending dose administration. As to ONT-093, the concentration was 10 mg/mL; and the dose volume was 2 mL/kg. Regarding saquinavir, the concentration was 20 mg/mL; and the dose volume was 1 mL/kg.

METHODS. A total of 16 male Sprague Dawley rats were received from Charles River Canada (Saint-Constant, Quebec). Animals were approximately 9 weeks of age at arrival and weighed ca 312-340 g. Four males were randomly assigned to each of Groups 1, 2, 3 and 4. On the day of dosing and following an overnight fast, animals were weighed (ca 305-323 g) and were administered their respective combination of doses. Animals of Groups 1 and 3 received a single oral dose of ONT-093 (20 mg/kg) by gavage at a dose volume of 2 mL/kg. Animals of Groups 2 and 4 received a single oral dose of the vehicle by gavage at a dose volume of 2 ml/kg. Approximately 30 min following administration of either ONT-093 or the vehicle, all animals received a single oral dose of saquinavir (20 mg/kg) by gavage at a dose volume of 1 mL/kg.

Following administration of saquinavir, blood samples (ca 0.3 mL in EDTA) were obtained from all rats of Groups 1 and 2 by jugular venipuncture during anesthesia with isoflurane (Ohmeda Pharmaceutical Products) at each of the following time-points: pre-dose (prior to administration of ONT-093 or vehicle) and 0.25, 0.5, 1, 2, 4 and 8 hr post-dose. Following administration of saquinavir, CSF was obtained from all animals of Groups 3 and 4 at 30 min following dose administration. CSF was collected (in tubes containing no anticoagulant) from the cistema magna during anesthesia with isoflurane. Following CSF collection, the animals were sacrificed by exsanguination via puncture of the vena cava and the brain was harvested from all these rats. Immediately following collection, all samples (blood, CSF and brain) were placed on ice until further processing or storage. Blood samples were centrifuged (ca 3080 g at ca 4° C. for 10 min) and the resulting plasma samples were stored at ca −80° C. pending analysis. CSF samples and brains were stored at ca −80° C. pending analysis.

Plasma samples, CSF samples and brains were analyzed for unchanged saquinavir using an LC/MS assay. Pharmacokinetic analysis was performed on plasma concentrations using the PhAST Software Program, Version 2.2-00 (Phoenix International Life Sciences Inc.). The highest observable concentration was assumed to be the peak concentration (Cmax); Time to Cmax was denoted Tmax. The area under the plasma concentration versus time-curve from time zero to the last measurable concentration [AUC(tf] was calculated by the linear trapezoidal method. Where applicable, numerical values were subjected to calculation of group means and % CV. Student-T-test with Satterthwaite approximation was performed on AUC and Cmax values to compare ONT-093 treated animals versus vehicle treated animals using SAS software program, Version 6.12 (SAS Institute Inc., Cary, N.C.). Where values were below limit of quantification, zero was used for pharmacokinetic analysis. The bioavailability (F) of saquinavir when administered with ONT-093 was calculated relative to the AUC values obtained from the animals receiving saquinavir following administration of the vehicle. The relative bioavailability was calculated as follows:

F=(AUC_{ONT-093/saquinavir}/AUC_{vehicle/saquinavir})×100

RESULTS. Most rats (3 of 4 in Group 1, 2 of 4 in Group 2, 3 of 4 in Group 3 and 4 of 4 in Group 4) exhibited slight salivation immediately following administration of the saquinavir dose. This clinical sign was no longer observable approximately 30 to 60 min after it was first observed.

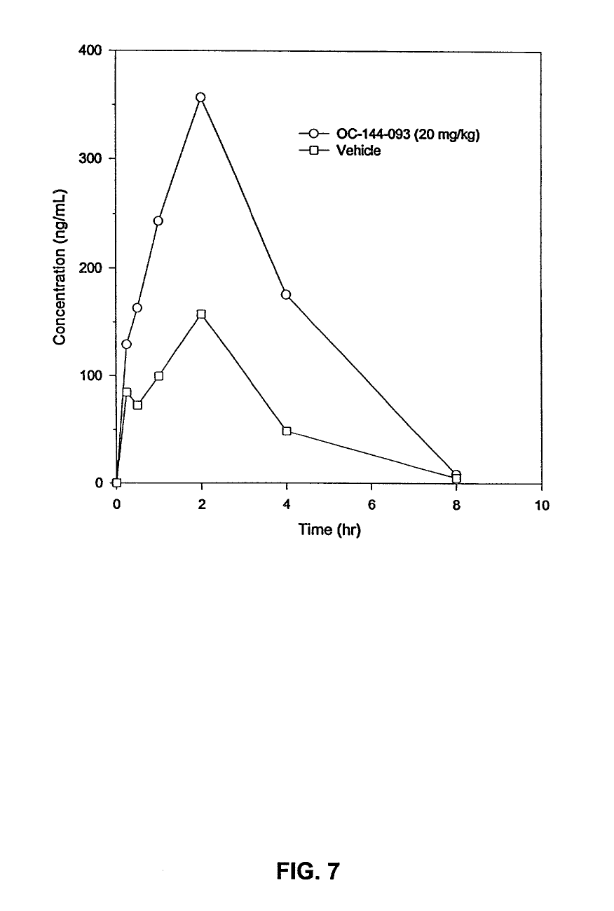

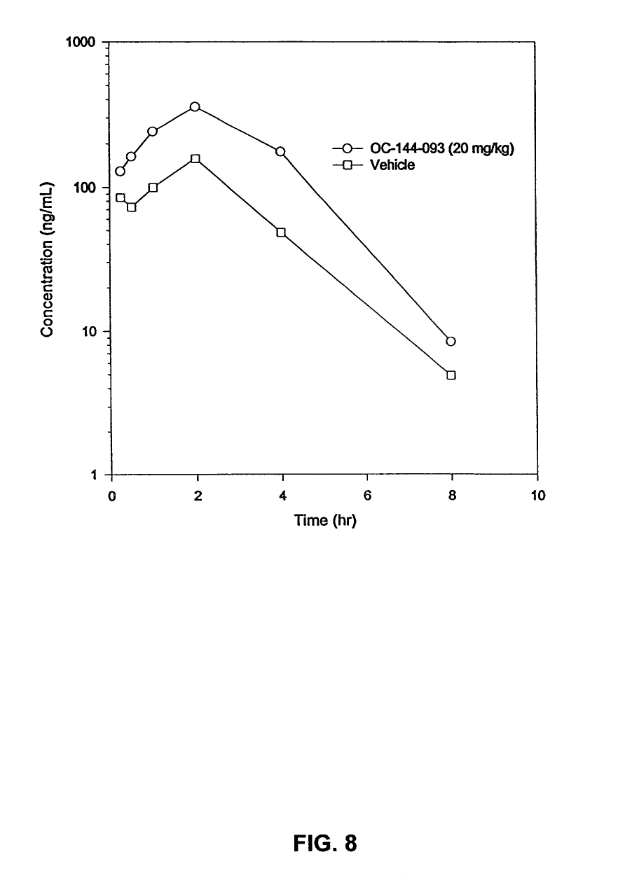

Pharmacokinetic parameters of saquinavir in plasma are presented in Table 5. Concentrations of saquinavir in plasma are presented in Table 6. Concentration of saquinavir in both CSF and brain are presented in Table 7. Linear and semi-log graphical representations of the concentration of saquinavir in plasma vs. time are presented in FIGS. 7 and 8, respectively.

Mean terminal phase half-life (t½) values for saquinavir were calculated to be 1.0 hr and 1.3 hr for Groups 1 and 2, respectively, following oral administration, suggesting a rapid elimination of the parent drug.

The mean Cmax value for saquinavir in plasma was significantly higher (p<0.0146) in animals receiving ONT-093 prior to the administration of saquinavir (Group 1: 374 ng/mL obtained at 2.3 hr post-dose) as compared with the mean Cmax value from animals receiving the vehicle only prior to saquinavir (Group 2: 181 ng/mL obtained at 1.6 hr). Similarly, the mean AUCinf value for saquinavir was significantly higher (ca 2.6 fold at p<0.0005) in animals that received the combination ONT-093/saquinavir (Group 1: 1365 ng-h/mL) as compared with those receiving the combination vehicle/saquinavir (Group 2: 524 ng-h/mL). This suggests that the administration of an oral 20 mg/kg dose of ONT-093, 30 min prior to dosing orally with saquinavir, significantly increases the bioavailability of saquinavir.

Concentrations of saquinavir in the cerebrospinal fluid were below the limit of detection in all rats with the exception of animal No. 3004 (pre-treated with ONT-093) which showed a concentration of 21.32 ng saquinavir/mL in the CSF. It is suspected that the presence of saquinavir in the cerebrospinal fluid of this animal resulted from a contamination with peripheral blood during collection. Detectable levels of saquinavir were measured in the brain tissues of all ONT-093 treated animals. However, the concentrations measured were low (0.30 to 1.16 ng/ml). Detectable levels of saquinavir were measured in one animal treated with the vehicle (1.40 ng/mL).

These results suggest that ONT-093 increases the bioavailability of saquinavir when administered orally 30 min prior to saquinavir but does not promote the passage of saquinavir across the blood-CSF barrier. The difference between brain tissue concentration of saquinavir in the ONT-093 treated animals versus vehicle treated animals suggests a possible increase in the blood/brain barrier permeability to saquinavir following administration of ONT-093.

TABLE 5
Pharmacokinetic Parameters of Saquinavir in Rat Plasma
When Administered Orally Following an Oral Dose of
ONT-093 (20 mg/kg) or Vehicle.

| | | AUC _{inf} | AUC ₀₋ₜ | Cmax | Tmax | | Thalf |
|---|---|---|---|---|---|---|---|
| Group | Treatment | (ng · hr/mL) | (ng · hr/mL) | (ng/mL) | (hr) | CL/F mL/kg · hr | (hr) |
| 1 | ONT-093 | 1365 | 1354^{a} | 374^{b} | 2.3 | 15070 | 1.0 |
| | | (239.2) | (240.8) | (138) | (1.26) | (3192.8) | (0.2) |
| 2 | Vehicle | 524 | 514 | 181 | 1.6 | 39227 | 1.3 |
| | | (97.7) | (100.3) | (35.6) | (0.88) | (7503.3) | (0.35) |

Calculations based on untruncated values
n = 4 for Groups 1 and 2
Values in parentheses are Standard Deviation (S.D.)
^{a}Significantly higher than Group 2 (P < 0.0005)
^{b}Significantly higher than Group 2 (P < 0.0146)

TABLE 6
Concentrations of Saquinavir (ng/mL) in Rat Plasma When Administered Orally Following
an Oral Dose of OC-144-093 (20 mg/kg) or Vehicle.

| | | 0.25 hr | 0.5 hr | 1 hr | 2 hr | 4 hr | 8 hr |
|---|---|---|---|---|---|---|---|
| Animal | Pre-Dose^{a} | Post-Dose^{b} | Post-Dose^{b} | Post-Dose^{b} | Post-Dose^{b} | Post-Dose^{b} | Post-Dose^{b} |
| 1001 | BLQ | 139.44 | 106.28 | 314.02 | 267.53 | 88.33 | 8.3 |
| 1002 | BLQ | 129.03 | 239.49 | 208.72 | 298.61 | 245.08 | 12.27 |
| 1003 | BLQ | 209.53 | 238.32 | 349.90 | 580.83 | 65.75 | 5.53 |
| 1004 | BLQ | 37.90 | 67.48 | 98.89 | 279.07 | 302.77 | 7.59 |
| Mean | NC | 128.95 | 162.89 | 242.88 | 356.51 | 175.48 | 8.42 |
| S.D. | NC | 70.45 | 89.19 | 113.16 | 150.09 | 116.45 | 2.82 |
| 2001 | BLQ | 22.96 | 12.96 | 44.65 | 204.81 | 87.04 | 5.13 |
| 2002 | BLQ | 92.17 | 30.68 | 46.84 | 144.91 | 37.3 | 3.77 |
| 2003 | BLQ | 4.55 | 32.84 | 99.75 | 158.29 | 37.83 | 7.98 |
| 2004 | BLQ | 218.79 | 213.44 | 206.82 | 120.91 | 31.75 | 2.78 |
| Mean | NC | 84.61 | 72.48 | 99.52 | 157.23 | 48.48 | 4.92 |
| S.D. | NC | 97.08 | 94.39 | 75.93 | 35.29 | 25.85 | 2.25 |

Calculations based on untruncated values
BLQ Below limit of quantitation
NC Not calculated
^{a}Pre-dose = Prior to administration of OC-144-093 or vehicle
^{b}Post-dose = Following administration of saquinavir

TABLE 7
Concentrations of Saquinavir (ng/mL) in Rat Cerebrospinal.
Fluid and Brain When Administered Orally Following an Oral
Dose of OC-144-093 (20 mg/kg) or Vehicle.

| | | Concentration in CSF* | Concentration in Brain |
|---|---|---|---|
| Group | Animal ID | (ng/mL) | (ng/mL) |
| 3 | 3001 | BLQ | 0.30 |
| | 3002 | BLQ | 1.14 |
| | 3003 | BLQ | 1.16 |
| | 3004 | 21.32^{a} | 0.90 |
| 4 | 4001 | BLQ | BLQ |
| | 4002 | BLQ | 1.40 |
| | 4003 | BLQ | BLQ |
| | 4004 | BLQ | BLQ |

Concentrations at 30 min following administration of the saquinavir dose
BLQ Below limit of quantification
^{a}CSF suspected to have been contaminated with peripheral blood during collection

Cell Lines, Animals and Reagents. CCRF-CEM and CEM/VLB1000 human lymphoma, SKOV3 and SK/VLB 1000 human ovarian carcinoma cells from V. Ling (Vancouver, BC) were grown in Alpha MEM with 2.0 mM glutamine and 10% FBS (Gemini BioProducts, Calabasas, Calif.), plus 1.0 μg/ml vinblastine sulfate for maintenance of drug resistance. MCF-7 and MCF-7/ADR (NCI, DCT Tumor Repository) and MCF-7/VP human breast carcinoma cells (E. Schneider, Albany, N.Y.) were grown in RPMI 1640 with 10% FBS. MES-SA and MES-SA/DX5 human uterine carcinoma cells (ATCC) were grown in McCoy's 5A with 10% FBS, plus 500 ng/ml doxorubicin for maintenance of drug resistance. MDA/LCC6 and mdr1 transduced MDA/LCC6^{MDR1} human breast carcinoma cells from R. Clarke (Georgetown University, Washington, DC) were grown in IMEM with 5% FBS. HCT-1 5 human colon carcinoma cells (ATCC) were grown in RPMI 1640 with 10% FBS. P-gp expression or lack thereof in cell lines was confirmed by FACS analysis using the monoclonal antibody MRK16 (Kamiya Biomedicals, Berkeley, Calif.). Nontransformed HISM human smooth muscle and primary CCD-986SK human skin cells (ATCC) were grown in DME with 10% FBS and Iscove's modified Dulbecco's medium with 10% FBS respectively.

Six to eight week old female BDF1 and SCID/RAG2 mice were obtained from Charles River Laboratories of Canada and the Joint Animal Care Facility at the BC Cancer Research Centre, respectively. MDA/LCC6 and MDA/LCC6^{MDR1} cells (10⁷ in 0.5 ml) were similarly propagated every 2-3 weeks in SCID/RAG2 mice. Cells were used between the 3^{rd} and 20^{th} passage. Animal studies were carried out with protocols approved by the BCCA/University of British Columbia Institutional Animal Care Committee and were performed in accordance with the Canadian Council on Animal Care Guidelines. The in vivo HCT-15 study was conducted by Serquest, a division of Southern Research Institute (Birmingham, Ala.), with young, adult female athymic NCr-nu mice.

Vinblastine, doxorubicin, daunomycin, and verapamil were purchased from Fluka (Ronkonkoma, N.Y.). Paclitaxel and Cyclosporin A were from Sigma (St. Louis, Mo.). Alamar Blue was from BioSource International (Camarillo, Calif.) and was used according to the manufacturer's instructions. The compound of Formula 2-free base for in vivo studies was synthesized by IRIX Pharmaceuticals, Inc. (Florence, S.C.). The material was 98% pure as judged by HPLC.

While the invention has been described in detail with reference to certain preferred embodiments thereof, it will be understood that modifications and variations are within the spirit and scope of that which is described and claimed.

What is claimed is:

1). A method of therapeutic and/or preventative treatment of a mammal that is afflicted or may become afflicted with a disease, comprising administration of an effective amount of a compound of Formula 1 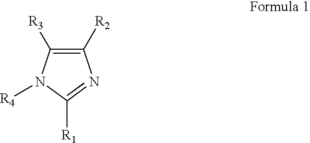
wherein the substituents R₁, R₂, R₃, and R₄ are defined as described in A and B below:
A. when R₁ is selected from the group consisting of:
(i) substituted C₁₋₁₁alkyl or substituted C₂₋₁₁alkenyl, wherein the substituents are selected from the group consisting of hydroxy, C₁₋₆alkyloxy; or
(ii) mono-, di-,and tri-substituted aryl-C₀₋₁₁alkyl wherein aryl is selected from the group consisting of phenyl, furyl, thienyl wherein the substituents are selected from the group consisting of:
(a) phenyl, trans-2-phenylethenyl, 2-phenylethynyl, 2-phenylethyl, or in which the said phenyl group is mono- or disubstituted with a member selected from the group consisting of hydroxy, halo, C₁₋₄ alkyl and C₁₋₄alkyloxy,
(b) substituted C₁₋₆alkyl, substituted C₂₋₆alkyloxy, substituted C₂₋₆alkylthio, substituted C₂₋₆alkoxycarbonyl, wherein the substituents are selected from the group consisting of C₁₋₆alkoxy, C₁₋₆alkylthio, or
(c) C₁₋₁₁CO₂R₅, C₁₋₁₁CONHR₅, trans-CH═CHCO₂R₅, or trans-CH═CHCONHR₅ wherein R₅ is C₁₋₁₁alkyl, or phenyl C₁₋₁₁alkyl, C₁₋₆alkoxycarbonylmethyleneoxy;
then R₂ and R₃ are each independently selected from the group consisting of mono-, di, and tri-substituted phenyl wherein the substituents are independently selected from:
(i) substituted C₁₋₆alkyl,
(ii) substituted C₁₋₆alkyloxy, C₃₋₆alkenyloxy, substituted C₃₋₆alkenyloxy,
(iii) substituted C₁₋₆alkyl-amino, di(substituted C₁₋₆alkyl)amino,
(iv) C₃₋₆alkenyl-amino, di(C₃₋₆alkenyl)amino, substituted C₃₋₆alkenyl-amino, di(substituted C₃₋₆alkenyl)amino,
(vi) pyrrolidino, piperidino, morpholino, imidazolyl, substituted imidazolyl, piperazino, N—C₁₋₆alkylpiperazino, N—C₃₋₆alkenylpiperazino, N—(C₁₋₆alkoxy C₁₋₆alkyl)piperazino, N—(C₁₋₆alkoxy C₃₋₆alkenyl)piperazino, N—(C₁₋₆alkylamino C₁₋₆alkyl)piperazino, N—(C₁₋₆alkylamino C₃₋₆alkenyl)piperazino,
wherein the substituents are selected from the group consisting of:
(a) hydroxy, C₁₋₆alkylalkoxy, C₁₋₆alkylamino,
(b) C₃₋₆alkenyloxy, C₃₋₆alkenylamino, or
(c) pyrrolidino, piperidino, morpholino, imidazolyl, substituted imidazolyl, piperazino, N—C₁₋₆alkylpiperazino, N—C₃₋₆alkenylpiperazino, N—(C₁₋₆alkoxy C₁₋₆alkyl)piperazino, N—(C₁₋₆alkoxy C₃₋₆alkenyl)piperazino, N—(C₁₋₆alkylamino C₁₋₆alkyl)piperazino, N—(C₁₋₆alkylamino C₃₋₆alkenyl)piperazino,
or R₂ and R₃ taken together forming an aryl group or substituted aryl, wherein the substituents are defined as above in (i)-(v);
and R₄ is selected from the group consisting of:
(i) hydrogen;
(ii) substituted C₁₋₁₁alkyl or C₂₋₁₁alkenyl wherein the substituents are independently selected from the group consisting of hydrogen, hydroxy, C₁₋₆alkyloxy, C₁₋₆alkylthio, C₁₋₆alkylamino, phenyl-C₁₋₆alkylamino, C₁₋₆alkoxycarbonyl; or
(iii) substituted aryl C₀₋₁₁alkyl wherein the aryl group is selected from phenyl, imidazolyl, furyl, thienyl in which the substituents are selected from A.(a-c); or
B. when R₁ is selected from the group consisting of:
Mono-,di-, and tri-substituted aryl-C₀₋₆alkyl wherein aryl is selected from the group consisting of phenyl, thienyl, and the substituents are selected from the group consisting of:
(a) trans-2-substituted benzimidazolylethenyl, trans-2-substituted benzoxazolylethenyl, trans-2-substituted benzthiazolylethenyl, in which the substituents are selected from the group consisting of hydrogen, hydroxy, halo, trihalomethyl, C₁₋₄alkyl and C₁₋₄alkyloxy, C₁₋₄alkyloxycarbonyl, C₁₋₄alkylamino, di(C₁₋₄alkyl)amino, C₃₋₆alkenylamino, di(C₃₋₆alkenyl)amino, C₁₋₄alkyloxy-C₁₋₄alkylamino, substituted C₁₋₄alkyl and C₁₋₄alkyloxy, substituted C₁₋₄alkyloxycarbonyl, substituted C₁₋₄alkylamino, di(substituted C₁₋₄alkyl)amino, substituted C₃₋₆alkenylamino, di(substituted C₃₋₆alkenyl)amino, wherein the substituents are as defined above,
(b) trans-2-cyano ethenyl, trans-2-alkylsulfonyl ethenyl, trans-2-alkenylsulfonyl ethenyl, trans-2-substituted alkylsulfonyl ethenyl, trans-2-substituted alkenylsulfonyl ethenyl, in which the substituents are defined above,
(c) C₁₋₆CO₂R₅, trans-CH═CHCO₂R₅, C₁₋₆CONHR₅, or trans-CH═CHCONHR₅, wherein R₅ is C₁₋₆alkoxy C₂₋₆alkyl, amino C₂₋₆alkyl, C₁₋₆alkylamino C₂₋₆alkyl, di(C₁₋₆alkyl)amino C₂₋₆alkyl, C₁₋₆alkylthio C₂₋₆alkyl, substituted C₁₋₆alkoxy C₂₋₆alkyl, substituted C₁₋₆alkylamino C₂₋₆alkyl, di(substituted C₁₋₆alkyl)amino C₂₋₆alkyl, substituted C₁₋₆alkylthio C₂₋₆alkyl, in which the substituents are selected from the group consisting of pyrrolidino, piperidino, morpholino, piperazino, N—C₁₋₆alkylpiperazino, N—C₃₋₆alkenylpiperazino, N—(C₁₋₆alkoxy C₁₋₆alkyl)piperazino, N—(C₁₋₆alkoxy C₃₋₆alkenyl)piperazino, N—(C₁₋₆alkylamino C₁₋₆alkyl)piperazino, N—(C₁₋₆alkylamino C₃₋₆alkenyl)piperazino, imidazolyl, oxazolyl, thiazolyl,
(d) C₁₋₆CONR₆R₇, or trans-CH═CHCONR₆R₇, wherein R₆ and R₇ are independently selected from the group consisting of C₁₋₆alkyl, phenyl C₁₋₆alkyl, C₁₋₆alkoxycarbonylmethyleneoxy, hydroxy C₂₋₆alkyl, C₁₋₆alkyloxy C₂₋₆alkyl, amino C₂₋₆alkyl, C₁₋₆alkylamino C₂₋₆ alkyl, di(C₁₋₆alkyl)amino C₂₋₆alkyl, C₁₋₆alkylthio C₂₋₆alkyl, substituted C₁₋₆alkoxy C₂₋₆alkyl, substituted C₁₋₆alkylamino C₂₋₆alkyl, di(substituted C₁₋₆alkyl)amino C₂₋₆alkyl, substituted C₁₋₆alkylthio C₂₋₆alkyl, wherein the substituents are selected from the group consisting of pyrrolidino, piperidino, morpholino, piperazino, N—C₁₋₆alkylpiperazino, N—C₃₋₆alkenylpiperazino, N—(C₁₋₆alkoxy C₁₋₆alkyl)piperazino, N—(C₁₋₆alkoxy C₃₋₆alkenyl)piperazino, N—(C₁₋₆alkylamino C₁₋₆alkyl)piperazino, N—(C₁₋₆alkylamino C₃₋₆alkenyl)piperazino, imidazolyl, oxazolyl, thiazolyl,
(e) R₇C(O) C₁₋₆alkyl, R₇C(O) carbonyl C₂₋₆alkenyl, in which R₇ is defined as above [2(d)],
(f) HO—C₁₋₆alkyl-C₂₋₆alkenyl, R₇—O—C₁₋₆alkyl-C₂₋₆alkenyl, R₇NH—C₁₋₆alkyl-C₂₋₆alkenyl, R₆R₇N—C₁₋₆alkyl-C₂₋₆alkenyl, R₇NH—C(O)—O—C₁₋₆alkyl-C₂₋₆alkenyl, R₆R₇N—C(O)—O—C₁₋₆alkyl-C₂₋₆alkenyl, R₇O—C(O)—O—C₁₋₆alkyl-C₂₋₆alkenyl, R₇—C(O)—O—C₁₋₆alkyl-C₂₋₆alkenyl, wherein R₆ and R₇ is defined as above [2(d)],
(g) R₇—O—C₀₋₃alkyl-C₃₋₆cycloalkan-1-yl, R₇NH—C₀₋₃alkyl-C₃₋₆cycloalkan-1-yl, R₆R₇N—C₀₋₃alkyl-C₃₋₆cycloalkan-1-yl, R₇NH—C(O)—O—C₀₋₃C₃₋₆cycloalkan-1-yl, R₆R₇N—C(O)—O—C₀₋₃alkyl-C₃₋₆cycloalkan-1-yl, R₇O—C(O)—O—C₀₋₃alkyl-C₃₋₆cycloalkan-1-yl, R₇—C(O)—O—C₀₋₃alkyl-C₃₋₆cycloalkan-1-yl, R₇O—C(O)—C₀₋₃alkyl-C₃₋₆cycloalkan-1-yl, wherein R₇ and is defined as above [2(d)];
then R₂ and R₃ are each independently selected from the group consisting of:
(1) hydrogen, halo, trihalomethyl, C₁₋₆alkyl, substituted C₁₋₆alkyl, C₁₋₆alkenyl, substituted C₁₋₆alkenyl, C₁₋₆alkyloxy, substituted C₁₋₆alkyloxy, C₃₋₆alkenyloxy, substituted C₃₋₆alkenyloxy, C₁₋₆alkylamino, substituted C₁₋₆alkylamino, C₃₋₆alkenylamino, substituted C₃₋₆alkenylamino,
(2) mono-, di-, and tri-substituted phenyl wherein the substituents are independently selected from:
(v) halo, trifluoromethyl, substituted C₁₋₆alkyl,
(vi) C₁₋₆alkyloxy, substituted C₁₋₆alkyloxy, C₃₋₆alkenyloxy, substituted C₃₋₆alkenyloxy,
(vii) C₁₋₆alkyl-amino, di(C₁₋₆alkyl)amino, substituted C₁₋₆alkyl-amino, di(substituted C₁₋₆alkyl)amino, C₃₋₆alkenyl-amino, di(C₃₋₆alkenyl)amino, substituted C₃₋₆alkenyl-amino, di(substituted C₃₋₆alkenyl)amino, or
(viii) pyrrolidino, piperidino, morpholino, imidazolyl, substituted imidazolyl, piperazino, N—C₁₋₆alkylpiperazino, N—C₃₋₆alkenylpiperazino, N—(C₁₋₆alkoxy C₁₋₆alkyl)piperazino, N—(C₁₋₆alkoxy C₃₋₆alkenyl)piperazino, N—(C₁₋₆alkylamino C₁₋₆alkyl)piperazino, N—(C₁₋₆alkylamino C₃₋₆alkenyl)piperazino,
wherein the substituents are selected from the group consisting of:
(a) hydrogen, hydroxy, halo, trifluoromethyl,
(b) C₁₋₆alkylalkoxy, C₁₋₆alkylamino, C₁₋₆alkylthio,
(c) C₃₋₆alkenyloxy, C₃₋₆alkenylamino, C₃₋₆alkenylthio, or
(d) pyrrolidino, piperidino, morpholino, imidazolyl, substituted imidazolyl, piperazino, N—C₁₋₆alkylpiperazino, N—C₃₋₆alkenylpiperazino, N—(C₁₋₆alkoxy C₁₋₆alkyl)piperazino, N—(C₁₋₆alkoxy C₃₋₆alkenyl)piperazino, N—(C₁₋₆alkylamino C₁₋₆alkyl)piperazino, N—(C₁₋₆alkylamino C₃₋₆alkenyl)piperazino;
with the proviso that at least one of R₂ and R₃ group be selected from
[B (2)] and the phenyl and the substituents be selected from (ii)-(v) above; or R₂ and R₃ taken together forming an aryl group or substituted aryl, wherein the substituents are defined as above in (i)-(iv);
and R₄ is selected from the group consisting of:
(a) hydrogen;
(b) substituted C₁₋₁₁alkyl or C₂₋₁₁alkenyl wherein the substituents are independently selected from the group consisting of hydrogen, hydroxy, C₁₋₆alkyloxy, C₁₋₆alkylthio, C₁₋₆alkylamino, phenyl-C₁₋₆alkylamino, C₁₋₆alkoxycarbonyl and the substituents are selected from (ii)-(iv); or
(c) aryl C₀₋₁₁alkyl wherein the aryl group is selected from phenyl, imidazolyl, furyl, thienyl
by steps comprising:
(a) choosing a pharmaceutically-active agent that is in the form of a free compound or its pharmaceutically-acceptable pro-drug, metabolite, analogue, derivative, solvate or salt, and that is selected from the group consisting of agents that (i) bind to or are substrates for P-gp, (ii) are taxane analogues, and/or (iii) are inhibitors of tubulin disassembly; and
(b) choosing a regimen of dosage frequency and amount of the pharmaceutically-active agent for such mammal that is therapeutically effective in the absence of the compound of Formula 1, taking into account the systemic toxicity of such pharmaceutically-active agent; and
(c) substantially increasing such dosage frequency or amount of the pharmaceutically-active agent to a toxicity-protected dosage, taking into account the protection against such systemic toxicity provided by such compound of Formula 1; and
(d) administering to such mammal (i) an effective amount of the compound of Formula 1 in the form of a free compound or its pharmaceutically-acceptable pro-drug, metabolite, analogue, derivative, solvate or salt; and (ii) such toxicity-protected dosage of such pharmaceutically-active agent.

2). The method of claim 1, in which such dosage frequency of the pharmaceutically-active agent is substantially increased for a given indication.

3). The method of claim 1, in which such dosage amount of the pharmaceutically-active agent is substantially increased for a given indication.

4). The method of claim 3, in which such toxicity-protected dosage amount is at least about 25% greater than such effective dosage for a given indication.

5). The method of claim 4, in which such toxicity-protected dosage amount is at least about 50% greater than such effective dosage for a given indication.

6). The method of claim 5, in which such toxicity-protected dosage amount is at least about 100% greater than such effective dosage for a given indication.

7). The method of claim 3, in which such toxicity-protected dosage amount is about 50% to about 100% greater than such effective dosage for a given indication.

8). The method of claim 1, in which such dosage frequency and amount of the pharmaceutically-active agent are substantially increased for a given indication.

9). The method of claim 1, in which such pharmaceutically-active agent is parenterally administered.

10). The method of claim 1, in which such pharmaceutically-active agent is orally administered.

11). The method of claim 1, in which the compound of Formula 1 is parenterally administered.

12). The method of claim 1, in which the compound of Formula 1 is orally administered.

13). The method of claim 1, in which the pharmaceutically-active agent and the compound of Formula 1 are topically administered.

14). The method of claim 1, in which the compound of Formula 1 is administered prior to administration of the pharmaceutically-active agent.

15). The method of claim 1, in which the pharmaceutically-active agent is administered prior to administration of the compound of Formula 1.

16). The method of claim 1, in which the compound of Formula 1 and the pharmaceutically-active agent substantially are simultaneously administered.

17). The method of claim 1, in which the compound of Formula 1 and the pharmaceutically-active agent are administered together in a combined dosage form.

18). The method of claim 1, in which the compound of Formula 1 and the pharmaceutically-active agent are independently administered in separate dosage forms.

19). The method of claim 1 in which the disease is characterized by the intrinsic presence of multi-drug resistance.

20). The method of claim 1 in which the disease is characterized by the potential to acquire multi-drug resistance.

21). The method of claim 1, in which such pharmaceutically-active agent comprises at least one agent in the form of a free compound or its pharmaceutically-acceptable pro-drug, metabolite, analogue, derivative, solvate or salt selected from the group consisting of: taxanes, epothilones, discodermolide, eleutherobin, sarcodictyins, laulimalides, vinca alkaloids, anthracyclines, camptothecins, epipodophyllotoxins, methotrexate, angiotensin converting enzyme (ACE) inhibitors, human immunodeficiency virus protease inhibitors, antibiotics, calcium channel antagonists, β-blockers, HMG-CoA reductase inhibitors, immunosuppressive agents, opiates, fluoroquinolones, macrolide antibiotics, aminoglycoside antibiotics, antihistamines, anti-epileptic agents, anti-malarial agents, and dopamine agonists.

22). The method of claim 1, in which such pharmaceutically-active agent comprises at least one agent in the form of a free compound or its pharmaceutically-acceptable pro-drug, metabolite, analogue, derivative, solvate or salt selected from the group consisting of: Abeta1-40 (β-amyloid); Abeta1-42 (β-amyloid); Acebutolol; Dactinomycin; Adefovir; Adrenaline; Epinephrine; Albuterol; Salbutamol; Aldosterone; Amikacin; Amitriptyline; Amprenavir; Astemizole; Atorvastatin; Aureobasidin A; Azasetron; Azathioprine; Azidopine; Azithromycin; Bilirubin; Bisantrene; Bunitrolol; Burroughs Wellcome (“BW”) 1019W91; BW 1288U89; BW 1351W91; BW 1379W91; Calcein-AM; Carbamazepine; Carvedilol; Celiprolol; Cerivastatin; Chloroquine; Chlorpromazine; Cimetidine; Clarithromycin; Colchicine; Corticosterone; Cyclosporine; Cyclosporine metabolite AM1; Cytosine arabinoside (cytarabine); Daunorubicin; Debrisoquine; 13-OH-4′-Deoxy-4′-iododoxorubicin; Dexamethasone; Digitoxin; Digoxin; αMethyl-Digoxin; β-acetyl Digoxin; Dihydroindolizino[7,6,5-kl]acridinium chloride; Diltiazem; desacetyl Diltiazem; Dipyridamole; Docetaxel; Domperidone; Doxorubicin; DPDE [D-penicillamine(2,5)]-enkephalin]; D-Penicillamine; Ebastine; Eletriptan; Emetine; Epirubicin; Erythromycin; Estradiol-17-β-D-glucuronide; Etoposide; Felodipine; Fentanyl; Fexofenadine; Flavopiridol; Fluconazole; Fluvastatin; Furosemide; Gemtuzumab ozogamicin; Glibenclamide; Glyburide; Gramicidin D; Grepafloxacin; Hoechst 33342; Hydrocortisone (cortisol); Bayer BAY59-8862 (Indena IDN-5109 paclitaxel analog); Imatinib (Gleevec); Interleukin-2; Interleukin-4; Indinavir; Interferon 2B; Interferon-γ-1B; Irinotecan (CPT-11); Isoniazid; Ivermectin; Labetalol; Dilevalol; L-Dopa (levodopa); Levofloxacin; Loperamide; Loratadine; Losartan; Lovastatin; Mefloquine; Melphalan; Methadone; Methamphetamine; Methotrexate; Methylprednisolone; Mibefradil; Miltefosine; Mitomycin C; Mitoxantrone; Monensin; Morphine; Morphine-6-glucuronide; Moxidectin; MPP+ (1-Methyl-4-phenylpyridium); Nadolol; Naringin; Nelfinavir; Neostigmine; Nicardipine; Nonylphenol ethoxylate; Nortriptyline; Octreotide; Omeprazole; Ondansetron; Paclitaxel; Phenytoin; 2-amino-1-methyl-6-phenylimidazo[4,5-b]pyridine (PhlP); Phosphatidylcholine; Phosphatidylethanolamine; Pirarubicin; Platelet Activating Factor; Plicamycin (Mithramycin); Prazosin; Pristinamycins; Propantheline; Propranolol; PSC833; Puromycin; Quinidine; Quinine; Ranitidine; Reserpine; Retinoic acid; Ritonavir; Saquinavir; Simvastatin; Sirolimus; Somatropin; Sparfloxacin; Tacrolimus; Talinol; Tc-Sestamibi; Terfenadine; Tetracycline; Thapsigargin; Timolol; Tobramycin; Topotecan; Trimethoprim; UK-224,671; Vecuronium; Verapamil; Verapamil metabolite (D-617); Verapamil metabolite (D-620); Vinblastine; Vincristine; Vindesine; and Vinorelbine.

23). The method of claim 21, in which such pharmaceutically-active agent comprises a taxane in the form of a free compound or its pharmaceutically-acceptable pro-drug, metabolite, analogue, derivative, solvate or salt.

24). The method of claim 23, in which such pharmaceutically-active agent comprises paclitaxel in the form of a free compound or its pharmaceutically-acceptable pro-drug, metabolite, analogue, derivative, solvate or salt.

25). The method of claim 24, in which such paclitaxel is parenterally administered during a treatment session and such toxicity-protected dosage is about 100 mg/m² to about 675 mg/m² per treatment session.

26). The method of claim 25, in which such toxicity-protected dosage is about 350 mg/m² to about 675 mg/m² per treatment session.

27). The method of claim 24, in which such paclitaxel is orally administered during a treatment session and such toxicity-protected dosage is about 125 mg to about 1200 mg per treatment session.

28). The method of claim 27, in which such toxicity-protected dosage is about 550 mg to about 1200 mg per treatment session.

29). The method of claim 23, in which such pharmaceutically-active agent comprises docetaxel in the form of a free compound or its pharmaceutically-acceptable pro-drug, metabolite, analogue, derivative, solvate or salt.

30). The method of claim 29, in which such docetaxel is parenterally administered during a treatment session and such toxicity-protected dosage is about 100 mg/m² to about 675 mg/m² per treatment session.

31). The method of claim 30, in which such toxicity-protected dosage is about 350 mg/m² to about 675 mg/m² per treatment session.

32). The method of claim 29, in which such docetaxel is orally administered during a treatment session and such toxicity-protected dosage is about 125 mg to about 1200 mg per treatment session.

33). The method of claim 32, in which such toxicity-protected dosage is about 550 mg to about 1200 mg per treatment session.

34). The method of claim 22, in which such pharmaceutically-active agent comprises saquinavir in the form of a free compound or its pharmaceutically-acceptable pro-drug, metabolite, analogue, derivative, solvate or salt.

35). The method of claim 34, in which such saquinavir is parenterally administered during a treatment session and such toxicity-protected dosage is about 13 mg/kg to about 39 mg/kg per treatment session.

36). The method of claim 35, in which such toxicity-protected dosage is about 19 mg/kg to about 39 mg/kg per treatment session.

37). The method of claim 34, in which such saquinavir is orally administered during a treatment session and such toxicity-protected dosage is about 600 mg to about 2400 mg per treatment session.

38). The method of claim 37, in which such toxicity-protected dosage is about 1200 mg to about 2400 mg per treatment session.

39). The method of claim 1 in which the disease is chronic and the pharmaceutically-active agent is administered to the mammal on a long-term basis.

40). The method of claim 1 in which the compound of Formula 1 is selected from the group consisting of: (2-[4-(3-ethoxy-1-propenyl)phenyl]-4,5-bis(4-(2-propylamino)phenyl)-1H-imidazole; 2-[4-(3-ethoxy-trans-1-propen-1-yl)phenyl]-4,5-bis (4-N,N-diethylaminophenyl) imidazole; 2-[4-(3-ethoxy-trans-1-propen-1-yl)phenyl]-4-(4-N,N-diethylaminophenyl)-5-(4-N-methylaminophenyl) imidazole; 2-[4-(3-methoxy-trans-1-propen-1-yl)phenyl]-4,5-bis (4-pyrrolidinophenyl) imidazole; 2-[4-(3-ethoxy-trans-1-propen-1-yl)phenyl]-4,5-bis (4-pyrrolidinophenyl) imidazole; 2-[4-(3-ethoxy-trans-1-propen-1-yl)phenyl]-4-(4-N-dimethylaminophenyl)-5-(4-pyrrolidinophenyl) imidazole; 2-[4-(3-ethoxy-trans-1-propen-1-yl)phenyl]-4-(4-N-methylaminophenyl)-5-(4-pyrrolidino-phenyl) imidazole; 2-[4-(3-ethoxy-trans-1-propen-1-yl)phenyl]-4,5-bis (4-N-morpholinophenyl) imidazole; 2-[4-(3-ethoxy-trans-1-propen-1-yl)phenyl]-4-(4-N-dimethylaminophenyl)-5-(4-N-morpholinophenyl) imidazole; 2-[4-(3ethoxy-trans-1-propen-1-yl)phenyl]-4-(4-N-methylaminophenyl)-5-(4-N-morpholinophenyl) imidazole; and 2-[4-(3ethoxy-trans-1-propen-1-yl)phenyl]-4-(4-N-methylaminophenyl)-5-(4-N-isopropylaminophenyl) imidazole.

41). The method of claim 40 in which the compound of Formula 1 has the following formula 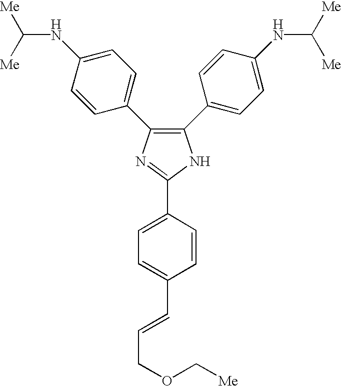
in the form of a free compound or as its pharmaceutically-acceptable pro-drug, metabolite, analogue, derivative or salt.

42). The method of claim 41 in which the compound of Formula 1 is in the form of a mesylate salt.

43). The method of claim 1 in which the disease comprises a disease of at least one of the following:
(a) an organ, including a: breast, lung, prostate, kidney, ovary, uterus, liver, pancreas, adrenal gland or
(b) the epithelial, gastric, intestinal, exocrine, endocrine, lymphatic, hematopoietic, genitourinary, colorectal, or central nervous system, or
(c) head, neck or skin tissue.

44). The method of claim 1 in which the disease is a disorder of the central nervous system.

45). The method of claim 44 in which the disease is epilepsy.

46). The method of claim 44 in which the disease is a cognitive disorder.

47). The method of claim 44 in which the disease is Alzheimer's disease.

48). The method of claim 44 in which the disease is Parkinson's disease.

49). The method of claim 1 in which the disease is a viral, bacterial, fungal, or parasitic infection.

50). The method of claim 49 in which the disease is human immunodeficiency virus.

51). The method of claim 1 in which the disease is psoriasis.

52). The method of claim 1 in which the disease is organ failure requiring an organ transplantation under conditions to prevent tissue rejection.

53). The method of claim 1 in which the mammal is a human.

54). In a method of therapeutic treatment of a mammal for a cell-proliferative disorder by administration of an effective amount of a compound of Formula 1 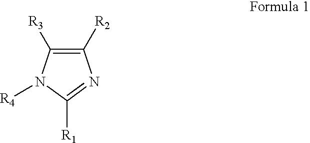
wherein the substituents R₁, R₂, R₃, and R₄ are defined as described in A and B below:
A. when R₁ is selected from the group consisting of:
(i) substituted C₁₋₁₁alkyl or substituted C₂₋₁₁alkenyl, wherein the substituents are selected from the group consisting of hydroxy, C₁₋₆alkyloxy; or
(ii) mono-, di-,and tri-substituted aryl-C₀₋₁₁alkyl wherein aryl is selected from the group consisting of phenyl, furyl, thienyl wherein the substituents are selected from the group consisting of:
(a) phenyl, trans-2-phenylethenyl, 2-phenylethynyl, 2-phenylethyl, or in which the said phenyl group is mono- or disubstituted with a member selected from the group consisting of hydroxy, halo, C₁₋₄alkyl and C₁₋₄alkyloxy,
(b) substituted C₁₋₆alkyl, substituted C₂₋₆alkyloxy, substituted C₂₋₆alkylthio, substituted C₂₋₆alkoxycarbonyl, wherein the substituents are selected from the group consisting of C₁₋₆alkoxy, C₁₋₆alkylthio, or
(c) C₁₋₁₁CO₂R₅, C₁₋₁₁CONHR₅, trans-CH═CHCO₂R₅, or trans-CH═CHCONHR₅ wherein R₅is C₁₋₁₁alkyl, or phenyl C₁₋₁₁alkyl, C₁₋₆alkoxycarbonylmethyleneoxy;
then R₂ and R₃ are each independently selected from the group consisting of mono-, di, and tri-substituted phenyl wherein the substituents are independently selected from:
(i) substituted C₁₋₆alkyl,
(ii) substituted C₁₋₆alkyloxy, C₃₋₆alkenyloxy, substituted C₃₋₆alkenyloxy,
(iii) substituted C₁₋₆alkyl-amino, di(substituted C₁₋₆alkyl)amino,
(iv) C₃₋₆alkenyl-amino, di(C₃₋₆alkenyl)amino, substituted C₃₋₆alkenyl-amino, di(substituted C₃₋₆alkenyl)amino,
(v) pyrrolidino, piperidino, morpholino, imidazolyl, substituted imidazolyl, piperazino, N—C₁₋₆alkylpiperazino, N—C₃₋₆alkenylpiperazino, N—(C₁₋₆alkoxy C₁₋₆alkyl)piperazino, N—(C₁₋₆alkoxy C₃₋₆alkenyl)piperazino, N—(C₁₋₆alkylamino C₁₋₆alkyl)piperazino, N—(C₁₋₆alkylamino C₃₋₆alkenyl)piperazino,
wherein the substituents are selected from the group consisting of:
(a) hydroxy, C₁₋₆alkylalkoxy, C₁₋₆alkylamino,
(b) C₃₋₆alkenyloxy, C₃₋₆alkenylamino, or
(c) pyrrolidino, piperidino, morpholino, imidazolyl, substituted imidazolyl, piperazino, N—C₁₋₆alkylpiperazino, N—C₃₋₆alkenylpiperazino, N—(C₁₋₆alkoxy C₁₋₆alkyl)piperazino, N—(C₁₋₆alkoxy C₃₋₆alkenyl)piperazino, N—(C₁₋₆alkylamino C₁₋₆alkyl)piperazino, N—(C₁₋₆alkylamino C₃₋₆alkenyl)piperazino,
or R₂ and R₃ taken together forming an aryl group or substituted aryl, wherein the substituents are defined as above in (i)-(v);
and R₄ is selected from the group consisting of:
(i) hydrogen;
(ii) substituted C₁₋₁₁alkyl or C₂₋₁₁alkenyl wherein the substituents are independently selected from the group consisting of hydrogen, hydroxy, C₁₋₆alkyloxy, C₁₋₆alkylthio, C₁₋₆alkylamino, phenyl-C₁₋₆alkylamino, C₁₋₆alkoxycarbonyl; or
(iii) substituted aryl C₀₋₁₁alkyl wherein the aryl group is selected from phenyl, imidazolyl, furyl, thienyl in which the substituents are selected from A.(a-c); or
B. when R₁ is selected from the group consisting of:
Mono-,di-, and tri-substituted aryl-C₀₋₆alkyl wherein aryl is selected from the group consisting of phenyl, thienyl, and the substituents are selected from the group consisting of:
(a) trans-2-substituted benzimidazolylethenyl, trans-2-substituted benzoxazolylethenyl, trans-2-substituted benzthiazolylethenyl, in which the substituents are selected from the group consisting of hydrogen, hydroxy, halo, trihalomethyl, C₁₋₄alkyl and C₁₋₄alkyloxy, C₁₋₄alkyloxycarbonyl, C₁₋₄alkylamino, di(C₁₋₄alkyl)amino, C₃₋₆alkenylamino, di(C₃₋₆alkenyl)amino, C₁₋₄alkyloxy-C₁₋₄alkylamino, substituted C₁₋₄alkyl and C₁₋₄alkyloxy, substituted C₁₋₄alkyloxycarbonyl, substituted C₁₋₄alkylamino, di(substituted C₁₋₄alkyl)amino, substituted C₃₋₆alkenylamino, di(substituted C₃₋₆alkenyl)amino, wherein the substituents are as defined above,
(b) trans-2-cyano ethenyl, trans-2-alkylsulfonyl ethenyl, trans-2-alkenylsulfonyl ethenyl, trans-2-substituted alkylsulfonyl ethenyl, trans-2-substituted alkenylsulfonyl ethenyl, in which the substituents are defined above,
(c) C₁₋₆CO₂R₅, trans-CH═CHCO₂R₅, C₁₋₆CONHR₅, or trans-CH═CHCONHR₅, wherein R₅ is C₁₋₆alkoxy C₂₋₆alkyl, amino C₂₋₆alkyl, C₁₋₆alkylamino C₂₋₆alkyl, di(C₁₋₆alkyl)amino C₂₋₆alkyl, C₁₋₆alkylthio C₂₋₆alkyl, substituted C₁₋₆alkoxy C₂₋₆alkyl, substituted C₁₋₆alkylamino C₂₋₆alkyl, di(substituted C₁₋₆alkyl)amino C₂₋₆alkyl, substituted C₁₋₆alkylthio C₂₋₆alkyl, in which the substituents are selected from the group consisting of pyrrolidino, piperidino, morpholino, piperazino, N—C₁₋₆alkylpiperazino, N—C₃₋₆alkenylpiperazino, N—(C₁₋₆alkoxy C₁₋₆alkyl)piperazino, N—(C₁₋₆alkoxy C₃₋₆alkenyl)piperazino, N—(C₁₋₆alkylamino C₁₋₆alkyl)piperazino, N—(C₁₋₆alkylamino C₃₋₆alkenyl)piperazino, imidazolyl, oxazolyl, thiazolyl,
(d) C₁₋₆CONR₆R₇, or trans-CH═CHCONR₆R₇, wherein R₆ and R₇ are independently selected from the group consisting of C₁₋₆alkyl, phenyl C₁₋₆alkyl, C₁₋₆alkoxycarbonylmethyleneoxy, hydroxy C₂₋₆alkyl, C₁₋₆alkyloxy C₂₋₆alkyl, amino C₂₋₆alkyl, C₁₋₆alkylamino C₂₋₆alkyl, di(C₁₋₆alkyl)amino C₂₋₆alkyl, C₁₋₆alkylthio C₂₋₆alkyl, substituted C₁₋₆alkoxy C₂₋₆alkyl, substituted C₁₋₆alkylamino C₂₋₆alkyl, di(substituted C₁₋₆alkyl)amino C₂₋₆alkyl, substituted C₁₋₆alkylthio C₂₋₆alkyl, wherein the substituents are selected from the group consisting of pyrrolidino, piperidino, morpholino, piperazino, N—C₁₋₆alkylpiperazino, N—C₃₋₆alkenylpiperazino, N—(C₁₋₆alkoxy C₁₋₆alkyl)piperazino, N—(C₁₋₆alkoxy C₃₋₆alkenyl)piperazino, N—(C₁₋₆alkylamino C₁₋₆alkyl)piperazino, N—(C₁₋₆alkylamino C₃₋₆alkenyl)piperazino, imidazolyl, oxazolyl, thiazolyl,
(e) R₇ C(O) C₁₋₆alkyl, R₇C(O) carbonyl C₂₋₆alkenyl, in which R₇ is defined as above [2(d)],
(f) HO—C₁₋₆alkyl-C₂₋₆alkenyl, R₇—O—C₁₋₆alkyl-C₂₋₆alkenyl, R₇NH—C₁₋₆alkyl-C₂₋₆alkenyl, R₆R₇N—C₁₋₆alkyl-C₂₋₆alkenyl, R₇NH—C(O)—O—C₁₋₆alkyl-C₂₋₆alkenyl, R₆R₇N—C(O)—O—C₁₋₆alkyl-C₂₋₆alkenyl, R₇O—C(O)—O—C₁₋₆alkyl-C₂₋₆alkenyl, R₇—C(O)—O—C₁₋₆alkyl-C₂₋₆alkenyl, wherein R₆ and R₇ is defined as above [2(d)],
(g) R₇—O—C₀₋₃alkyl-C₃₋₆cycloalkan-1-yl, R₇NH—C₀₋₃alkyl-C₃₋₆cycloalkan-1-yl, R₆R₇N—C₀₋₃alkyl-C₃₋₆cycloalkan-1-yl, R₇NH—C(O)—O—C₀₋₃C₃₋₆cycloalkan-1-yl, R₆R₇N—C(O)—O—C₀₋₃alkyl-C₃₋₆cycloalkan-1-yl, R₇O—C(O)—O—C₀₋₃alkyl-C₃₋₆cycloalkan-1-yl, R₇—C(O)—O—C₀₋₃alkyl-C₃₋₆cycloalkan-1-yl, R₇O—C(O)—C₀₋₃alkyl-C₃₋₆cycloalkan-1-yl, wherein R₇ and is defined as above [2(d)];
then R₂ and R₃ are each independently selected from the group consisting of:
(1) hydrogen, halo, trihalomethyl, C₁₋₆alkyl, substituted C₁₋₆alkyl, C₁₋₆alkenyl, substituted C₁₋₆alkenyl, C₁₋₆alkyloxy, substituted C₁₋₆alkyloxy, C₃₋₆alkenyloxy, substituted C₃₋₆alkenyloxy, C₁₋₆alkylamino, substituted C₁₋₆alkylamino, C₃₋₆alkenylamino, substituted C₃₋₆alkenylamino,
(2) mono-, di-, and tri-substituted phenyl wherein the substituents are independently selected from:
(i) halo, trifluoromethyl, substituted C₁₋₆alkyl,
(ii) C₁₋₆alkyloxy, substituted C₁₋₆alkyloxy, C₃₋₆alkenyloxy, substituted C₃₋₆alkenyloxy,
(iii) C₁₋₆alkyl-amino, di(C₁₋₆alkyl)amino, substituted C₁₋₆alkyl-amino, di(substituted C₁₋₆alkyl)amino, C₃₋₆alkenyl-amino, di(C₃₋₆alkenyl)amino, substituted C₃₋₆alkenyl-amino, di(substituted C₃₋₆alkenyl)amino, or
(iv) pyrrolidino, piperidino, morpholino, imidazolyl, substituted imidazolyl, piperazino, N—C₁₋₆alkylpiperazino, N—C₃₋₆alkenylpiperazino, N—(C₁₋₆alkoxy C₁₋₆alkyl)piperazino, N—(C₁₋₆alkoxy C₃₋₆alkenyl)piperazino, N—(C₁₋₆alkylamino C₁₋₆alkyl)piperazino, N—(C₁₋₆alkylamino C₃₋₆alkenyl)piperazino,
wherein the substituents are selected from the group consisting of
(a) hydrogen, hydroxy, halo, trifluoromethyl,
(b) C₁₋₆alkylalkoxy, C₁₋₆alkylamino, C₁₋₆alkylthio,
(c) C₃₋₆alkenyloxy, C₃₋₆alkenylamino, C₃₋₆alkenylthio, or
(d) pyrrolidino, piperidino, morpholino, imidazolyl, substituted imidazolyl, piperazino, N—C₁₋₆alkylpiperazino, N—C₃₋₆alkenylpiperazino, N—(C₁₋₆alkoxy C₁₋₆alkyl)piperazino, N—(C₁₋₆alkoxy C₃₋₆alkenyl)piperazino, N—(C₁₋₆alkylamino C₁₋₆alkyl)piperazino, N—(C₁₋₆alkylamino C₃₋₆alkenyl)piperazino;
with the proviso that at least one of R₂ and R₃ group be selected from
[B (2)] and the phenyl and the substituents be selected from (ii)-(v) above; or R₂ and R₃ taken together forming an aryl group or substituted aryl, wherein the substituents are defined as above in (i)-(iv);
and R₄ is selected from the group consisting of:
(a) hydrogen;
(b) substituted C₁₋₁₁alkyl or C₂₋₁₁alkenyl wherein the substituents are independently selected from the group consisting of hydrogen, hydroxy, C₁₋₆alkyloxy, C₁₋₆alkylthio, C₁₋₆alkylamino, phenyl-C₁₋₆alkylamino, C₁₋₆alkoxycarbonyl and the substituents are selected from (ii)-(iv); or
(c) aryl C₀₋₁₁alkyl wherein the aryl group is selected from phenyl, imidazolyl, furyl, thienyl
by steps comprising:
(a) choosing an anti-cell-proliferative therapeutic agent that is in the form of a free compound or its pharmaceutically-acceptable pro-drug, metabolite, analogue, derivative, solvate or salt, and that is selected from the group consisting of agents that (i) bind to or are substrates for P-gp, (ii) are taxane analogues, and/or (iii) are inhibitors of tubulin disassembly; and
(b) choosing a regimen of dosage frequency and amount of the anti-cell-proliferative therapeutic agent for such mammal that is therapeutically effective in the absence of the compound of Formula 1, taking into account the systemic toxicity of such anti-cell-proliferative therapeutic agent; and
(c) substantially increasing such dosage frequency or amount of the anti-cell-proliferative therapeutic agent to a toxicity-protected dosage, taking into account the protection against such systemic toxicity provided by such compound of Formula 1; and
(d) administering to such mammal (i) an effective amount of the compound of Formula 1 in the form of a free compound or its pharmaceutically-acceptable pro-drug, metabolite, analogue, derivative, solvate or salt; and (ii) such toxicity-protected dosage of such anti-cell-proliferative therapeutic agent.

55). The method of claim 54, in which such dosage frequency of the anti-cell-proliferative therapeutic agent is substantially increased for a given indication.

56). The method of claim 55, in which such effective regimen includes administration of the anti-cell-proliferative therapeutic agent at a frequency of about once every three weeks during a course of treatment, and such frequency is increased to at least about once every two weeks during such course of treatment.

57). The method of claim 55, in which such effective regimen includes administration of the anti-cell-proliferative therapeutic agent at a frequency of about once every week during a course of treatment, and such frequency is increased to at least about once every three days during such course of treatment.

58). The method of claim 54, in which such dosage amount of the anti-cell-proliferative therapeutic agent for a given indication is substantially increased for a given indication.

59). The method of claim 58, in which such toxicity-protected dosage amount is at least about 25% greater than such effective dosage for a given indication.

60). The method of claim 58, in which such toxicity-protected dosage amount is at least about 50% greater than such effective dosage for a given indication.

61). The method of claim 58, in which such toxicity-protected dosage amount is at least about 100% greater than such effective dosage for a given indication.

62). The method of claim 58, in which such toxicity-protected dosage amount is about 50% to about 100% greater than such effective dosage for a given indication.

63). The method of claim 58, in which such dosage frequency and amount of the anti-cell-proliferative therapeutic agent are substantially increased for a given indication.

64). The method of claim 54, in which such anti-cell-proliferative therapeutic agent is parenterally administered.

65). The method of claim 54, in which such anti-cell-proliferative therapeutic agent is orally administered.

66). The method of claim 54, in which the compound of Formula 1 is parenterally administered.

67). The method of claim 54, in which the compound of Formula 1 is orally administered.

68). The method of claim 54, in which such anti-cell-proliferative therapeutic agent and the compound of Formula 1 are topically administered.

69). The method of claim 54, in which the compound of Formula 1 is administered prior to administration of the anti-cell-proliferative therapeutic agent.

70). The method of claim 54, in which the anti-cell-proliferative therapeutic agent is administered prior to administration of the compound of Formula 1.

71). The method of claim 54, in which the compound of Formula 1 and the anti-cell-proliferative therapeutic agent substantially are simultaneously administered.

72). The method of claim 54, in which the compound of Formula 1 and the anti-cell-proliferative therapeutic agent are administered together in a combined dosage form.

73). The method of claim 54, in which the compound of Formula 1 and the anti-cell-proliferative therapeutic agent are independently administered in separate dosage forms.

74). The method of claim 54 in which such cells either do not express P-gp, do not express P-gp in all cells, or do not express P-gp at levels sufficient to manifest complete multi-drug resistance.

75). The method of claim 54 in which such cells have not previously been exposed to an anti-cell-proliferative therapeutic agent.

76). The method of claim 54 in which such cells express P-gp and manifest multi-drug resistance.

77). The method of claim 54 in which the effective dosage is determined based upon the chemotherapeutic index of such anti-cell-proliferative therapeutic agent, and treatment with such compound of Formula 1 increases such chemotherapeutic index.

78). The method of claim 54, in which such anti-cell-proliferative therapeutic agent comprises at least one agent in the form of a free compound or its pharmaceutically-acceptable pro-drug, metabolite, analogue, derivative, solvate or salt selected from the group consisting of: taxanes, epothilones, discodermolide, eleutherobin, sarcodictyins, laulimalides, vinca alkaloids, anthracyclines, camptothecins, and epipodophyllotoxins.

79). The method of claim 54, in which such anti-cell-proliferative therapeutic agent comprises at least one agent in the form of a free compound or its pharmaceutically-acceptable pro-drug, metabolite, analogue, derivative, solvate or salt selected from the group consisting of: paclitaxel, docetaxel, vinblastine, vincristine, vinorelbine, doxorubicin, daunorubicin, etoposide, topotecan, dactinomycin, plicamycin (mithramycin), mitomycin, verapamil, cytosine arabinoside (cytarabine), methotrexate, and irinotecan (CPT-11).

80). The method of claim 78, in which such anti-cell-proliferative therapeutic agent comprises a taxane in the form of a free compound or its pharmaceutically-acceptable pro-drug, metabolite, analogue, derivative, solvate or salt.

81). The method of claim 80, in which such anti-cell-proliferative therapeutic agent comprises paclitaxel in the form of a free compound or its pharmaceutically-acceptable pro-drug, metabolite, analogue, derivative, solvate or salt.

82). The method of claim 81, in which such paclitaxel is parenterally administered during a treatment session and such toxicity-protected dosage is about 100 mg/m² to about 675 mg/m² per treatment session.

83). The method of claim 82, in which such toxicity-protected dosage is about 350 mg/m² to about 675 mg/m² per treatment session.

84). The method of claim 81, in which such paclitaxel is orally administered during a treatment session and such toxicity-protected dosage is about 125 mg to about 1200 mg per treatment session.

85). The method of claim 84, in which such toxicity-protected dosage is about 550 mg to about 1200 mg per treatment session.

86). The method of claim 82, in which such treatment session comprises administering: (a) about 35 mg to about 700 mg of the compound of Formula 1 at about 8 to about 16 hours before such paclitaxel administration; (b) about 35 mg to about 700 mg of the compound of Formula 1 at about 1 to about 3 hours before or with such paclitaxel administration; and (c) about 35 mg to about 700 mg of the compound of Formula 1 at about 6 to about 10 hours after such paclitaxel administration.

87). The method of claim 86, in which such treatment session comprises administering: (a) about 50 mg to about 500 mg of the compound of Formula 1 at about 8 to about 16 hours before such paclitaxel administration; (b) about 50 mg to about 500 mg of the compound of Formula 1 at about 1 to about 3 hours before or with such paclitaxel administration; and (c) about 50 mg to about 500 mg of the compound of Formula 1 at about 6 to about 10 hours after such paclitaxel administration.

88). The method of claim 84, in which such treatment session comprises administering: (a) about 100 mg to about 750 mg of the compound of Formula 1 at about 8 to about 16 hours before such paclitaxel administration; (b) about 100 mg to about 750 mg of the compound of Formula 1 at about 1 to about 3 hours before or with such paclitaxel administration; and (c) about 100 mg to about 750 mg of the compound of Formula 1 at about 6 to about 10 hours after such paclitaxel administration.

89). The method of claim 88, in which such treatment session comprises administering: (a) about 300 mg to about 500 mg of the compound of Formula 1 at about 8 to about 16 hours before such paclitaxel administration; (b) about 300 mg to about 500 mg of the compound of Formula 1 at about 1 to about 3 hours before or with such paclitaxel administration; and (c) about 300 mg to about 500 mg of the compound of Formula 1 at about 6 to about 10 hours after such paclitaxel administration.

90). The method of claim 80, in which such pharmaceutically-active agent comprises docetaxel in the form of a free compound or its pharmaceutically-acceptable pro-drug, metabolite, analogue, derivative, solvate or salt.

91). The method of claim 90, in which such docetaxel is parenterally administered during a treatment session and such toxicity-protected dosage is about 100 mg/m² to about 675 mg/m² per treatment session.

92). The method of claim 91, in which such toxicity-protected dosage is about 350 mg/m² to about 675 mg/m² per treatment session.

93). The method of claim 90, in which such docetaxel is orally administered during a treatment session and such toxicity-protected dosage is about 125 mg to about 1200 mg per treatment session.

94). The method of claim 93, in which such toxicity-protected dosage is about 550 mg to about 1200 mg per treatment session.

95). The method of claim 91, in which such treatment session comprises administering: (a) about 35 mg to about 700 mg of the compound of Formula 1 at about 8 to about 16 hours before such docetaxel administration; (b) about 35 mg to about 700 mg of the compound of Formula 1 at about 1 to about 3 hours before or with such docetaxel administration; and (c) about 35 mg to about 700 mg of the compound of Formula 1 at about 6 to about 10 hours after such docetaxel administration.

96). The method of claim 95, in which such treatment session comprises administering: (a) about 50 mg to about 500 mg of the compound of Formula 1 at about 8 to about 16 hours before such docetaxel administration; (b) about 50 mg to about 500 mg of the compound of Formula 1 at about 1 to about 3 hours before or with such docetaxel administration; and (c) about 50 mg to about 500 mg of the compound of Formula 1 at about 6 to about 10 hours after such docetaxel administration.

97). The method of claim 93, in which such treatment session comprises administering: (a) about 100 mg to about 750 mg of the compound of Formula 1 at about 8 to about 16 hours before such docetaxel administration; (b) about 100 mg to about 750 mg of the compound of Formula 1 at about 1 to about 3 hours before or with such docetaxel administration; and (c) about 100 mg to about 750 mg of the compound of Formula 1 at about 6 to about 10 hours after such docetaxel administration.

98). The method of claim 97, in which such treatment session comprises administering: (a) about 300 mg to about 500 mg of the compound of Formula 1 at about 8 to about 16 hours before such docetaxel administration; (b) about 300 mg to about 500 mg of the compound of Formula 1 at about 1 to about 3 hours before or with such docetaxel administration; and (c) about 300 mg to about 500 mg of the compound of Formula 1 at about 6 to about 10 hours after such docetaxel administration.

99). The method of claim 54 in which the disease is chronic and the pharmaceutically-active agent is administered to the mammal on a long-term basis.

100). The method of claim 54 in which the compound of Formula 1 is selected from the group consisting of: (2-[4-(3-ethoxy-1-propenyl)phenyl]-4,5-bis(4-(2-propylamino)phenyl)-1H-imidazole; 2-[4-(3-ethoxy-trans-1-propen-1-yl)phenyl]-4,5-bis (4-N,N-diethylaminophenyl) imidazole; 2-[4-(3-ethoxy-trans-1-propen-1-yl)phenyl]-4-(4-N,N-diethylaminophenyl)-5-(4-N-methylaminophenyl) imidazole; 2-[4-(3-methoxy-trans-1-propen-1-yl)phenyl]-4,5-bis (4-pyrrolidinophenyl) imidazole; 2-[4-(3-ethoxy-trans-1-propen-1-yl)phenyl]-4,5-bis (4-pyrrolidinophenyl) imidazole; 2-[4-(3-ethoxy-trans-1-propen-1-yl)phenyl]-4-(4-N-dimethylaminophenyl)-5-(4-pyrrolidinophenyl) imidazole; 2-[4-(3-ethoxy-trans-1-propen-1-yl)phenyl]-4-(4-N-methylaminophenyl)-5-(4-pyrrolidino-phenyl) imidazole; 2-[4-(3-ethoxy-trans-1-propen-1-yl)phenyl]-4,5-bis (4-N-morpholinophenyl) imidazole; 2-[4-(3-ethoxy-trans-1-propen-1-yl)phenyl]-4-(4-N-dimethylaminophenyl)-5-(4-N-morpholinophenyl) imidazole; 2-[4-(3-ethoxy-trans-1-propen-1-yl)phenyl]-4-(4-N-methylaminophenyl)-5-(4-N-morpholinophenyl) imidazole; and 2-[4-(3-ethoxy-trans-1-propen-1-yl)phenyl]-4-4-N-methylaminophenyl)-5-(4-N-isopropylaminophenyl) imidazole.

101). The method of claim 100 in which the compound of Formula 1 has the following formula 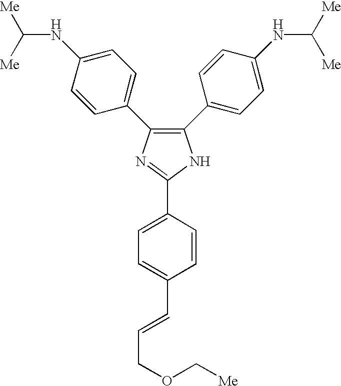
in the form of a free compound or as its pharmaceutically-acceptable pro-drug, metabolite, analogue, derivative, solvate or salt.

102). The method of claim 101 in which the compound of Formula 1 is in the form of a mesylate salt.

103). The method of claim 54 in which the cell proliferative disorder comprises a disease of at least one of the following:
(a) an organ, including a: breast, lung, prostate, kidney, ovary, uterus, liver, pancreas, adrenal gland or
(b) the epithelial, gastric, intestinal, exocrine, endocrine, lymphatic, hematopoietic, genitourinary, colorectal, or central nervous system, or
(c) head, neck or skin tissue.

104). The method of claim 54 in which the cell proliferative disorder is a neoplasm.

105). The method of claim 54 in which the cell proliferative disorder is a cancer.

106). The method of claim 105 in which the cancer is metastatic breast cancer.

107). The method of claim 54 in which the cell proliferative disorder is a tumor.

108). The method of claim 54 in which the cell proliferative disorder is a fibrotic disorder.

109). The method of claim 54 in which the cell proliferative disorder is acute myeloid leukemia.

110). The method of claim 1 in which the mammal is a human.

111). A pharmaceutical composition for oral administration of therapeutic treatment for a cell-proliferative disorder, comprising (a) a taxane in an amount exceeding about 550 milligrams, in the form of a free compound or its pharmaceutically-acceptable pro-drug, metabolite, analogue, derivative or salt, and (b) a toxicity-protecting amount of a compound of Formula 1 in the form of a free compound or its pharmaceutically-acceptable pro-drug, metabolite, analogue, derivative or salt.

112). The pharmaceutical composition of claim 111, comprising at least about 650 milligrams of a taxane in the form of a free compound or its pharmaceutically-acceptable pro-drug, metabolite, analogue, derivative or salt.

113). The pharmaceutical composition of claim 112, comprising at least about 775 milligrams of a taxane in the form of a free compound or its pharmaceutically-acceptable pro-drug, metabolite, analogue, derivative or salt.

114). The pharmaceutical composition of claim 111 in which the compound of Formula 1 is selected from the group consisting of: (2-[4-(3-ethoxy-1-propenyl)phenyl]-4,5-bis(4-(2-propylamino)phenyl)-1H-imidazole; 2-[4-(3-ethoxy-trans-1-propen-1-yl)phenyl]-4,5-bis (4-N,N-diethylaminophenyl) imidazole; 2-[4-(3-ethoxy-trans-1-propen-1-yl)phenyl]-4-(4-N,N-diethylaminophenyl)-5-(4-N-methylaminophenyl) imidazole; 2-[4-(3-methoxy-trans-1-propen-1-yl)phenyl]-4,5-bis (4-pyrrolidinophenyl) imidazole; 2-[4-(3-ethoxy-trans-1-propen-1-yl)phenyl]-4,5-bis (4-pyrrolidinophenyl) imidazole; 2-[4-(3-ethoxy-trans-1-propen-1-yl)phenyl]-4-(4-N-dimethylaminophenyl)-5-(4-pyrrolidinophenyl) imidazole; 2-[4-(3-ethoxy-trans-1-propen-1-yl)phenyl]-4-(4-N-methylaminophenyl)-5-(4-pyrrolidino-phenyl) imidazole; 2-[4-(3-ethoxy-trans-1-propen-1-yl)phenyl]-4,5-bis (4-N-morpholinophenyl) imidazole; 2-[4-(3-ethoxy-trans-1-propen-1-yl)phenyl]-4-(4-N-dimethylaminophenyl)-5-(4-N-morpholinophenyl) imidazole; 2-[4-(3-ethoxy-trans-1-propen-1-yl)phenyl]-4-(4-N-methylaminophenyl)-5-(4-N-morpholinophenyl) imidazole; and 2-[4-(3-ethoxy-trans-1-propen-1-yl)phenyl]-4-(4-N-methylaminophenyl)-5-(4-N-isopropylaminophenyl) imidazole.

115). The pharmaceutical composition of claim 111 in which the compound of Formula 1 has the following formula: 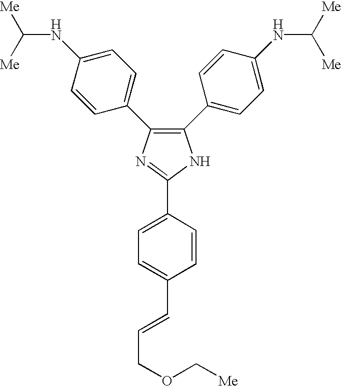
in the form of a free compound or as its pharmaceutically-acceptable pro-drug, metabolite, derivative or salt.

116). The composition of claim 111 in which the compound of Formula 1 is in the form of a mesylate salt.

117). A pharmaceutical composition for oral administration of therapeutic treatment for a cell-proliferative disorder, comprising (a) paclitaxel in an amount exceeding about 550 milligrams, in the form of a free compound or its pharmaceutically-acceptable pro-drug, metabolite, analogue, derivative or salt, and (b) a toxicity-protecting amount of a compound of Formula 1 in the form of a free compound or its pharmaceutically-acceptable pro-drug, metabolite, analogue, derivative or salt.

118). The pharmaceutical composition of claim 117, comprising at least about 650 milligrams of paclitaxel in the form of a free compound or its pharmaceutically-acceptable pro-drug, metabolite, analogue, derivative or salt.

119). The pharmaceutical composition of claim 118, comprising at least about 775 milligrams of paclitaxel in the form of a free compound or its pharmaceutically-acceptable pro-drug, metabolite, analogue, derivative or salt.

120). The pharmaceutical composition of claim 117 in which the compound of Formula 1 is selected from the group consisting of: (2-[4-(3-ethoxy-1-propenyl)phenyl]-4,5-bis(4-(2-propylamino)phenyl)-1H-imidazole; 2-[4-(3-ethoxy-trans-1-propen-1-yl)phenyl]-4,5-bis (4-N,N-diethylaminophenyl) imidazole; 2-[4-(3-ethoxy-trans-1-propen-1-yl)phenyl]-4-(4-N,N-diethylaminophenyl)-5-(4-N-methylaminophenyl) imidazole; 2-[4-(3-methoxy-trans-1-propen-1-yl)phenyl]-4,5-bis (4-pyrrolidinophenyl) imidazole; 2-[4-(3-ethoxy-trans-1-propen-1-yl)phenyl]-4,5-bis (4-pyrrolidinophenyl) imidazole; 2-[4-(3-ethoxy-trans-1-propen-1-yl)phenyl]-4-(4-N-dimethylaminophenyl)-5-(4-pyrrolidinophenyl) imidazole; 2-[4-(3-ethoxy-trans-1-propen-1-yl)phenyl]-4-(4-N-methylaminophenyl)-5-(4-pyrrolidino-phenyl) imidazole; 2-[4-(3-ethoxy-trans-1-propen-1-yl)phenyl]-4,5-bis (4-N-morpholinophenyl) imidazole; 2-[4-(3-ethoxy-trans-1-propen-1-yl)phenyl]-4-(4-N-dimethylaminophenyl)-5-(4-N-morpholinophenyl) imidazole; 2-[4-(3-ethoxy-trans-1-propen-1-yl)phenyl]-4-(4-N-methylaminophenyl)-5-(4-N-morpholinophenyl) imidazole; and 2-[4-(3-ethoxy-trans-1-propen-1-yl)phenyl]-4-(4-N-methylaminophenyl)-5-(4-N-isopropylaminophenyl) imidazole.

121). The pharmaceutical composition of claim 117 in which the compound of Formula 1 has the following formula 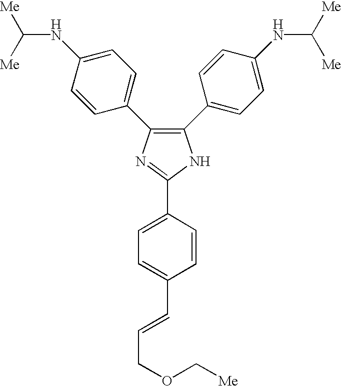
in the form of a free compound or as its pharmaceutically-acceptable pro-drug, metabolite, derivative or salt.

122). The composition of claim 117 in which the compound of Formula 1 is in the form of a mesylate salt.

123). A method of increasing the bioavailability of therapeutic and/or preventative treatment in a mammal, comprising administration to the mammal of an effective amount of a compound of Formula 1 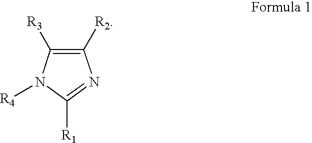
wherein the substituents R₁, R₂, R₃, and R₄ are defined as described in A and B below:
A. when R₁ is selected from the group consisting of:
(i) substituted C₁₋₁₁alkyl or substituted C₂₋₁₁alkenyl, wherein the substituents are selected from the group consisting of hydroxy, C₁₋₆alkyloxy; or
(ii) mono-, di-,and tri-substituted aryl-C₀₋₁₁alkyl wherein aryl is selected from the group consisting of phenyl, furyl, thienyl wherein the substituents are selected from the group consisting of:
(a) phenyl, trans-2-phenylethenyl, 2-phenylethynyl, 2-phenylethyl, or in which the said phenyl group is mono- or disubstituted with a member selected from the group consisting of hydroxy, halo, C₁₋₄alkyl and C₁₋₄alkyloxy,
(b) substituted C₁₋₆alkyl, substituted C₂₋₆alkyloxy, substituted C₂₋₆alkylthio, substituted C₂₋₆alkoxycarbonyl, wherein the substituents are selected from the group consisting of C₁₋₆alkoxy, C₁₋₆alkylthio, or
(c) C₁₋₁₁CO₂R₅, C₁₋₁₁CONHR₅, trans-CH═CHCO₂R₅, or trans-CH═CHCONHR₅ wherein R₅ is C₁₋₁₁, alkyl, or phenyl C₁₋₁₁alkyl, C₁₋₆alkoxycarbonylmethyleneoxy;
then R₂ and R₃ are each independently selected from the group consisting of mono-, di, and tri-substituted phenyl wherein the substituents are independently selected from:
(i) substituted C₁₋₆alkyl,
(ii) substituted C₁₋₆alkyloxy, C₃₋₆alkenyloxy, substituted C₃₋₆alkenyloxy,
(iii) substituted C₁₋₆alkyl-amino, di(substituted C₁₋₆alkyl)amino,
(iv) C₃₋₆alkenyl-amino, di(C₃₋₆alkenyl)amino, substituted C₃₋₆alkenyl-amino, di(substituted C₃₋₆alkenyl)amino,
(v) pyrrolidino, piperidino, morpholino, imidazolyl, substituted imidazolyl, piperazino, N—C₁₋₆alkylpiperazino, N—C₃₋₆alkenylpiperazino, N—(C₁₋₆alkoxy C₁₋₆alkyl)piperazino, N—(C₁₋₆alkoxy C₃₋₆alkenyl)piperazino, N—(C₁₋₆alkylamino C₁₋₆alkyl)piperazino, N—(C₁₋₆alkylamino C₃₋₆alkenyl)piperazino,
wherein the substituents are selected from the group consisting of:
(a) hydroxy, C₁₋₆alkylalkoxy, C₁₋₆alkylamino,
(b) C₃₋₆alkenyloxy, C₃₋₆alkenylamino, or
(c) pyrrolidino, piperidino, morpholino, imidazolyl, substituted imidazolyl, piperazino, N—C₁₋₆alkylpiperazino, N—C₃₋₆alkenylpiperazino, N—(C₁₋₆alkoxy C₁₋₆alkyl)piperazino, N—(C₁₋₆alkoxy C₃₋₆alkenyl)piperazino, N—(C₁₋₆alkylamino C₁₋₆alkyl)piperazino, N—(C₁₋₆alkylamino C₃₋₆alkenyl)piperazino,
or R₂ and R₃ taken together forming an aryl group or substituted aryl, wherein the substituents are defined as above in (i)-(v);
and R₄ is selected from the group consisting of:
(i) hydrogen;
(ii) substituted C₁₋₁₁alkyl or C₂₋₁₁alkenyl wherein the substituents are independently selected from the group consisting of hydrogen, hydroxy, C₁₋₆alkyloxy, C₁₋₆alkylthio, C₁₋₆alkylamino, phenyl-C₁₋₆alkylamino, C₁₋₆alkoxycarbonyl; or
(iii) substituted aryl C₀₋₁₁alkyl wherein the aryl group is selected from phenyl, imidazolyl, furyl, thienyl in which the substituents are selected from A.(a-c); or
B. when R₁ is selected from the group consisting of:
Mono-,di-, and tri-substituted aryl-C₀₋₆alkyl wherein aryl is selected from the group consisting of phenyl, thienyl, and the substituents are selected from the group consisting of:
(a) trans-2-substituted benzimidazolylethenyl, trans-2-substituted benzoxazolylethenyl, trans-2-substituted benzthiazolylethenyl, in which the substituents are selected from the group consisting of hydrogen, hydroxy, halo, trihalomethyl, C₁₋₄alkyl and C₁₋₄alkyloxy, C₁₋₄alkyloxycarbonyl, C₁₋₄alkylamino, di(C₁₋₄alkyl)amino, C₃₋₆alkenylamino, di(C₃₋₆alkenyl)amino, C₁₋₄alkyloxy-C₁₋₄alkylamino, substituted C₁₋₄alkyl and C₁₋₄alkyloxy, substituted C₁₋₄alkyloxycarbonyl, substituted C₁₋₄alkylamino, di(substituted C₁₋₄alkyl)amino, substituted C₃₋₆alkenylamino, di(substituted C₃₋₆alkenyl)amino, wherein the substituents are as defined above,
(b) trans-2-cyano ethenyl, trans-2-alkylsulfonyl ethenyl, trans-2-alkenylsulfonyl ethenyl, trans-2-substituted alkylsulfonyl ethenyl, trans-2-substituted alkenylsulfonyl ethenyl, in which the substituents are defined above,
(c) C₁₋₆CO₂R₅, trans-CH═CHCO₂R₅, C₁₋₆CONHR₅, or trans-CH═CHCONHR₅, wherein R₅ is C₁₋₆alkoxy C₂₋₆alkyl, amino C₂₋₆alkyl, C₁₋₆alkylamino C₂₋₆alkyl, di(C₁₋₆alkyl)amino C₂₋₆alkyl, C₁₋₆alkylthio C₂₋₆alkyl, substituted C₁₋₆alkoxy C₂₋₆alkyl, substituted C₁₋₆alkylamino C₂₋₆alkyl, di(substituted C₁₋₆alkyl)amino C₂₋₆alkyl, substituted C₁₋₆alkylthio C₂₋₆alkyl, in which the substituents are selected from the group consisting of pyrrolidino, piperidino, morpholino, piperazino, N—C₁₋₆alkylpiperazino, N—C₃₋₆alkenylpiperazino, N—(C₁₋₆alkoxy C₁₋₆alkyl)piperazino, N—(C₁₋₆alkoxy C₃₋₆alkenyl)piperazino, N—(C₁₋₆alkylamino C₁₋₆alkyl)piperazino, N—(C₁₋₆alkylamino C₃₋₆alkenyl)piperazino, imidazolyl, oxazolyl, thiazolyl,
(d) C₁₋₆CONR₆R₇, or trans-CH═CHCONR₆R₇, wherein R₆ and R₇ are independently selected from the group consisting of C₁₋₆alkyl, phenyl C₁₋₆alkyl, C₁₋₆alkoxycarbonylmethyleneoxy, hydroxy C₂₋₆alkyl, C₁₋₆alkyloxy C₂₋₆alkyl, amino C₂₋₆alkyl, C₁₋₆alkylamino C₂₋₆alkyl, di(C₁₋₆alkyl)amino C₂₋₆alkyl, C₁₋₆alkylthio C₂₋₆alkyl, substituted C₁₋₆alkoxy C₂₋₆alkyl, substituted C₁₋₆alkylamino C₂₋₆alkyl, di(substituted C₁₋₆alkyl)amino C₂₋₆alkyl, substituted C₁₋₆alkylthio C₂₋₆alkyl, wherein the substituents are selected from the group consisting of pyrrolidino, piperidino, morpholino, piperazino, N—C₁₋₆alkylpiperazino, N—C₃₋₆alkenylpiperazino, N—(C₁₋₆alkoxy C₁₋₆alkyl)piperazino, N—(C₁₋₆alkoxy C₃₋₆alkenyl)piperazino, N—(C₁₋₆alkylamino C₁₋₆alkyl)piperazino, N—(C₁₋₆alkylamino C₃₋₆alkenyl)piperazino, imidazolyl, oxazolyl, thiazolyl,
(e) R₇C(O)C₁₋₆alkyl, R₇C(O) carbonyl C₂₋₆alkenyl, in which R₇ is defined as above [2(d)],
(f) HO—C₁₋₆alkyl-C₂₋₆alkenyl, R₇—O—C₁₋₆alkyl-C₂₋₆alkenyl, R₇NH—C₁₋₆alkyl-C₂₋₆alkenyl, R₆R₇N—C₁₋₆alkyl-C₂₋₆alkenyl, R₇NH—C(O)—O—C₁₋₆alkyl-C₂₋₆alkenyl, R₆R₇N—C(O)—O—C₁₋₆alkyl-C₂₋₆alkenyl, R₇O—C(O)—O—C₁₋₆alkyl-C₂₋₆alkenyl, R₇—C(O)—O—C₁₋₆alkyl-C₂₋₆alkenyl, wherein R₆ and R₇ is defined as above [2(d)],
(g) R₇—O—C₀₋₃alkyl-C₃₋₆cycloalkan-1-yl, R₇NH—C₀₋₃alkyl-C₃₋₆cycloalkan-1-yl, R₆R₇N—C₀₋₃alkyl-C₃₋₆cycloalkan-1-yl, R₇NH—C(O)—O—C₀₋₃C₃₋₆cycloalkan-1-yl, R₆R₇N—C(O)—O—C₀₋₃alkyl-C₃₋₆cycloalkan-1-yl, R₇O—C(O)—O—C₀₋₃alkyl-C₃₋₆cycloalkan-1-yl, R₇—C(O)—O—C₀₋₃alkyl-C₃₋₆cycloalkan-1-yl, R₇O—C(O)—C₀₋₃alkyl-C₃₋₆cycloalkan-1-yl, wherein R₇ and is defined as above [2(d)];
then R₂ and R₃ are each independently selected from the group consisting of:
(1) hydrogen, halo, trihalomethyl, C₁₋₆alkyl, substituted C₁₋₆alkyl, C₁₋₆alkenyl, substituted C₁₋₆alkenyl, C₁₋₆alkyloxy, substituted C₁₋₆alkyloxy, C₃₋₆alkenyloxy, substituted C₃₋₆alkenyloxy, C₁₋₆alkylamino, substituted C₁₋₆alkylamino, C₃₋₆alkenylamino, substituted C₃₋₆alkenylamino,
(2) mono-, di-, and tri-substituted phenyl wherein the substituents are independently selected from:
(i) halo, trifluoromethyl, substituted C₁₋₆alkyl,
(ii) C₁₋₆alkyloxy, substituted C₁₋₆alkyloxy, C₃₋₆alkenyloxy, substituted C₃₋₆alkenyloxy,
(iii) C₁₋₆alkyl-amino, di(C₁₋₆alkyl)amino, substituted C₁₋₆alkyl-amino, di(substituted C₁₋₆alkyl)amino, C₃₋₆alkenyl-amino, di(C₃₋₆alkenyl)amino, substituted C₃₋₆alkenyl-amino, di(substituted C₃₋₆alkenyl)amino, or
(iv) pyrrolidino, piperidino, morpholino, imidazolyl, substituted imidazolyl, piperazino, N—C₁₋₆alkylpiperazino, N—C₃₋₆alkenylpiperazino, N—(C₁₋₆alkoxy C₁₋₆alkyl)piperazino, N—(C₁₋₆alkoxy C₃₋₆alkenyl)piperazino, N—(C₁₋₆alkylamino C₁₋₆alkyl)piperazino, N—(C₁₋₆alkylamino C₃₋₆alkenyl)piperazino,
wherein the substituents are selected from the group consisting of:
(a) hydrogen, hydroxy, halo, trifluoromethyl,
(b) C₁₋₆alkylalkoxy, C₁₋₆alkylamino, C₁₋₆alkylthio,
(c) C₃₋₆alkenyloxy, C₃₋₆alkenylamino, C₃₋₆alkenylthio, or
(d) pyrrolidino, piperidino, morpholino, imidazolyl, substituted imidazolyl, piperazino, N—C₁₋₆alkylpiperazino, N—C₃₋₆alkenylpiperazino, N—(C₁₋₆alkoxy C₁₋₆alkyl)piperazino, N—(C₁₋₆alkoxy C₃₋₆alkenyl)piperazino, N—(C₁₋₆alkylamino C₁₋₆alkyl)piperazino, N—(C₁₋₆alkylamino C₃₋₆alkenyl)piperazino;
with the proviso that at least one of R₂ and R₃ group be selected from
[B (2)] and the phenyl and the substituents be selected from (ii)-(v) above; or R₂ and R₃ taken together forming an aryl group or substituted aryl, wherein the substituents are defined as above in (i)-(iv);
and R₄ is selected from the group consisting of:
(a) hydrogen;
(b) substituted C₁₋₁₁alkyl or C₂₋₁₁alkenyl wherein the substituents are independently selected from the group consisting of hydrogen, hydroxy, C₁₋₆alkyloxy, C₁₋₆alkylthio, C₁₋₆alkylamino, phenyl-C₁₋₆alkylamino, C₁₋₆alkoxycarbonyl and the substituents are selected from (ii)-(iv); or
(c) aryl C₀₋₁₁alkyl wherein the aryl group is selected from phenyl, imidazolyl, furyl, thienyl
by steps comprising:
(a) choosing a pharmaceutically-active agent that is in the form of a free compound or its pharmaceutically-acceptable pro-drug, metabolite, analogue, derivative, solvate or salt, and that is selected from the group consisting of agents that (i) bind to or are substrates for P-gp, and/or (ii) are taxane analogues; and
(b) administering to such mammal an effective amount of the compound of Formula 1 in the form of a free compound or its pharmaceutically-acceptable pro-drug, metabolite, analogue, derivative, solvate or salt; and
(c) orally administering to such mammal an effective amount of such pharmaceutically-active agent.

124). The method of claim 123, in which the compound of Formula 1 is administered prior to administration of the pharmaceutically-active agent.

125). The method of claim 123, in which the pharmaceutically-active agent is administered prior to administration of the compound of Formula 1.

126). The method of claim 123, in which the compound of Formula 1 and the pharmaceutically-active agent substantially are simultaneously administered.

127). The method of claim 123, in which the compound of Formula 1 and the pharmaceutically-active agent are administered together in a combined dosage form.

128). The method of claim 123, in which the compound of Formula 1 and the pharmaceutically-active agent are independently administered in separate dosage forms.

129). The method of claim 123, comprising the following additional steps that precede steps (b) and (c):
(i) choosing a regimen of dosage frequency and amount of the pharmaceutically-active agent for such mammal that is therapeutically effective in the absence of the compound of Formula 1, taking into account the systemic toxicity of such pharmaceutically-active agent; and
(ii) substantially increasing such dosage frequency or amount of the pharmaceutically-active agent to a toxicity-protected dosage, taking into account the protection against such systemic toxicity provided by such compound of Formula 1.

130). The method of claim 129, in which such dosage frequency of the pharmaceutically-active agent is substantially increased for a given indication.

131). The method of claim 129, in which such dosage amount of the pharmaceutically-active agent is substantially increased for a given indication.

132). The method of claim 123 in which the disease is characterized by the intrinsic presence of multi-drug resistance.

133). The method of claim 123 in which the disease is characterized by the potential to acquire multi-drug resistance.

134). The method of claim 123, in which such pharmaceutically-active agent comprises at least one agent in the form of a free compound or its pharmaceutically-acceptable pro-drug, metabolite, analogue, derivative, solvate or salt selected from the group consisting of: taxanes, epothilones, discodermolide, eleutherobin, sarcodictyins, laulimalides, vinca alkaloids, anthracyclines, camptothecins, epipodophyllotoxins, methotrexate, angiotensin converting enzyme (ACE) inhibitors, human immunodeficiency virus protease inhibitors, antibiotics, calcium channel antagonists, β-blockers, HMG-CoA reductase inhibitors, immunosuppressive agents, opiates, fluoroquinolones, macrolide antibiotics, aminoglycoside antibiotics, antihistamines, anti-epileptic agents, anti-malarial agents, and dopamine agonists.

135). The method of claim 123, in which such pharmaceutically-active agent comprises at least one agent in the form of a free compound or its pharmaceutically-acceptable pro-drug, metabolite, analogue, derivative, solvate or salt selected from the group consisting of: Abeta1-40 (β-amyloid); Abeta1-42 (β-amyloid); Acebutolol; Dactinomycin; Adefovir; Adrenaline; Epinephrine; Albuterol; Salbutamol; Aldosterone; Amikacin; Amitriptyline; Amprenavir; Astemizole; Atorvastatin; Aureobasidin A; Azasetron; Azathioprine; Azidopine; Azithromycin; Bilirubin; Bisantrene; Bunitrolol; Burroughs Wellcome (“BW”) 1019W91; BW 1288U89; BW 1351W91; BW 1379W91; Calcein-AM; Carbamazepine; Carvedilol; Celiprolol; Cerivastatin; Chloroquine; Chlorpromazine; Cimetidine; Clarithromycin; Colchicine; Corticosterone; Cyclosporine; Cyclosporine metabolite AM1; Cytosine arabinoside (cytarabine); Daunorubicin; Debrisoquine; 13-OH-4-Deoxy-4′-iododoxorubicin; Dexamethasone; Digitoxin; Digoxin; αMethyl-Digoxin; β-acetyl Digoxin; Dihydroindolizino[7,6,5-kl]acridinium chloride; Diltiazem; desacetyl Diltiazem; Dipyridamole; Docetaxel; Domperidone; Doxorubicin; DPDE [D-penicillamine(2,5)]-enkephalin]; D-Penicillamine; Ebastine; Eletriptan; Emetine; Epirubicin; Erythromycin; Estradiol-17-β-D-glucuronide; Etoposide; Felodipine; Fentanyl; Fexofenadine; Flavopiridol; Fluconazole; Fluvastatin; Furosemide; Gemtuzumab ozogamicin; Glibenclamide; Glyburide; Gramicidin D; Grepafloxacin; Hoechst 33342; Hydrocortisone (cortisol); Bayer BAY59-8862 (Indena IDN-5109 paclitaxel analog); Imatinib (Gleevec); Interleukin-2; Interleukin-4; Indinavir; Interferon 2B; Interferon-γ-1B; Irinotecan (CPT-11); Isoniazid; Ivermectin; Labetalol; Dilevalol; L-Dopa (levodopa); Levofloxacin; Loperamide; Loratadine; Losartan; Lovastatin; Mefloquine; Melphalan; Methadone; Methamphetamine; Methotrexate; Methylprednisolone; Mibefradil; Miltefosine; Mitomycin C; Mitoxantrone; Monensin; Morphine; Morphine-6-glucuronide; Moxidectin; MPP+ (1-Methyl-4-phenylpyridium); Nadolol; Naringin; Nelfinavir; Neostigmine; Nicardipine; Nonylphenol ethoxylate; Nortriptyline; Octreotide; Omeprazole; Ondansetron; Paclitaxel; Phenytoin; 2-amino-1-methyl-6-phenylimidazo[4,5-b]pyridine (PhlP); Phosphatidylcholine; Phosphatidylethanolamine; Pirarubicin; Platelet Activating Factor; Plicamycin (Mithramycin); Prazosin; Pristinamycins; Propantheline; Propranolol; PSC833; Puromycin; Quinidine; Quinine; Ranitidine; Reserpine; Retinoic acid; Ritonavir; Saquinavir; Simvastatin; Sirolimus; Somatropin; Sparfloxacin; Tacrolimus; Talinol; Tc-Sestamibi; Terfenadine; Tetracycline; Thapsigargin; Timolol; Tobramycin; Topotecan; Trimethoprim; UK-224,671; Vecuronium; Verapamil; Verapamil metabolite (D-617); Verapamil metabolite (D-620); Vinblastine; Vincristine; Vindesine; and Vinorelbine.

136). The method of claim 134, in which such pharmaceutically-active agent comprises a taxane in the form of a free compound or its pharmaceutically-acceptable pro-drug, metabolite, analogue, derivative, solvate or salt.

137). The method of claim 136, in which such pharmaceutically-active agent comprises paclitaxel in the form of a free compound or its pharmaceutically-acceptable pro-drug, metabolite, analogue, derivative, solvate or salt.

138). The method of claim 137, in which the dosage administered of such paclitaxel during a treatment session is about 120 mg to about 1200 mg.

139). The method of claim 138, in which such dosage is about 550 mg to about 1200 mg per treatment session.

140). The method of claim 136, in which such pharmaceutically-active agent comprises docetaxel in the form of a free compound or its pharmaceutically-acceptable pro-drug, metabolite, analogue, derivative, solvate or salt.

141). The method of claim 139, in which the dosage administered of such docetaxel during a treatment session is about 120 mg to about 1200 mg.

142). The method of claim 141, in which such dosage is about 550 mg to about 1200 mg per treatment session.

143). The method of claim 135, in which such pharmaceutically-active agent comprises saquinavir in the form of a free compound or its pharmaceutically-acceptable pro-drug, metabolite, analogue, derivative, solvate or salt.

144). The method of claim 143, in which the dosage administered of such saquinavir during a treatment session is about 600 mg to about 2400 mg per treatment session.

145). The method of claim 144, in which such dosage is about 1200 mg to about 2400 mg per treatment session.

146). The method of claim 123 in which the disease is chronic and the pharmaceutically-active agent is administered to the mammal on a long-term basis.

147). The method of claim 123 in which the compound of Formula 1 is selected from the group consisting of: (2-[4-(3-ethoxy-1-propenyl)phenyl]-4,5-bis(4-(2-propylamino)phenyl)-1H-imidazole; 2-[4-(3-ethoxy-trans-1-propen-1-yl)phenyl]-4,5-bis (4-N,N-diethylaminophenyl) imidazole; 2-[4-(3-ethoxy-trans-1-propen-1-yl)phenyl]-4-(4-N,N-diethylaminophenyl)-5-(4-N-methylaminophenyl) imidazole; 2-[4-(3-methoxy-trans-1-propen-1-yl)phenyl]-4,5-bis (4-pyrrolidinophenyl) imidazole; 2-[4-(3-ethoxy-trans-1-propen-1-yl)phenyl]-4,5-bis (4-pyrrolidinophenyl) imidazole; 2-[4-(3-ethoxy-trans-1-propen-1-yl)phenyl]-4-(4-N-dimethylaminophenyl)-5-(4-pyrrolidinophenyl) imidazole; 2-[4-(3-ethoxy-trans-1-propen-1-yl)phenyl]-4-(4-N-methylaminophenyl)-5-(4-pyrrolidino-phenyl) imidazole; 2-[4-(3-ethoxy-trans-1-propen-1-yl)phenyl]-4,5-bis (4-N-morpholinophenyl) imidazole; 2-[4-(3-ethoxy-trans-1-propen-1-yl)phenyl]-4-(4-N-dimethylaminophenyl)-5-(4-N-morpholinophenyl) imidazole; 2-[4-(3-ethoxy-trans-1-propen-1-yl)phenyl]-4-(4-N-methylaminophenyl)-5-(4-N-morpholinophenyl) imidazole; and 2-[4-(3-ethoxy-trans-1-propen-1-yl)phenyl]-4-(4-N-methylaminophenyl)-5-(4-N-isopropylaminophenyl) imidazole.

148). The method of claim 147 in which the compound of Formula 1 has the following formula: 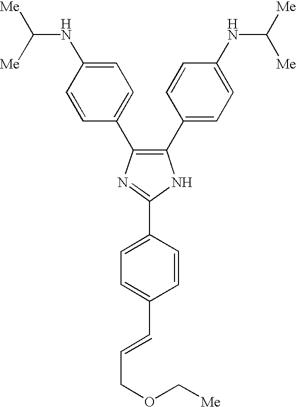
in the form of a free compound or as its pharmaceutically-acceptable pro-drug, metabolite, analogue, derivative or salt.

149). The method of claim 148 in which the compound of Formula 1 is in the form of a mesylate salt.

150). The method of claim 123 in which the disease comprises a disease of at least one of the following:
(a) an organ, including a: breast, lung, prostate, kidney, ovary, uterus, liver, pancreas, adrenal gland or
(b) the epithelial, gastric, intestinal, exocrine, endocrine, lymphatic, hematopoietic, genitourinary, colorectal, or central nervous system, or
(c) head, neck or skin tissue.

151). The method of claim 123 in which the disease is a disorder of the central nervous system.

152). The method of claim 151 in which the disease is epilepsy.

153). The method of claim 151 in which the disease is a cognitive disorder.

154). The method of claim 151 in which the disease is Alzheimer's disease.

155). The method of claim 151 in which the disease is Parkinson's disease.

156). The method of claim 123 in which the disease is a viral, bacterial, fungal, or parasitic infection.

157). The method of claim 156 in which the disease is human immunodeficiency virus.

158). The method of claim 123 in which the disease is organ failure requiring an organ transplantation under conditions to prevent tissue rejection.

159). The method of claim 123 in which the disease is a cell proliferative disorder and the pharmaceutically-active compound is an anti-cell-proliferative therapeutic agent.

160). The method of claim 159 in which the cell proliferative disorder is a neoplasm.

161). The method of claim 159 in which the cell proliferative disorder is a cancer.

162). The method of claim 159 in which the cell proliferative disorder is metastatic breast cancer.

163). The method of claim 159 in which the cell proliferative disorder is a tumor.

164). The method of claim 159 in which the cell proliferative disorder is a fibrotic disorder.

165). The method of claim 159 in which the cell proliferative disorder is acute myeloid leukemia.

166). The method of claim 159 in which such cells have not previously been exposed to an anti-cell-proliferative therapeutic agent.

167). The method of claim 159 in which such cells express P-gp and manifest multi-drug resistance.

168). The method of claim 159, in which such anti-cell-proliferative therapeutic agent comprises at least one agent in the form of a free compound or its pharmaceutically-acceptable pro-drug, metabolite, analogue, derivative, solvate or salt selected from the group consisting of: taxanes, epothilones, discodermolide, eleutherobin, sarcodictyins, laulimalides, vinca alkaloids, anthracyclines, camptothecins, and epipodophyllotoxins.

169). The method of claim 159, in which such anti-cell-proliferative therapeutic agent comprises at least one agent in the form of a free compound or its pharmaceutically-acceptable pro-drug, metabolite, analogue, derivative, solvate or salt selected from the group consisting of: paclitaxel, docetaxel, vinblastine, vincristine, vinorelbine, doxorubicin, daunorubicin, etoposide, topotecan, dactinomycin, plicamycin (mithramycin), mitomycin, verapamil, cytosine arabinoside (cytarabine), methotrexate, and irinotecan (CPT-11).

170). The method of claim 137, in which such treatment session comprises administering: (a) about 100 mg to about 750 mg of the compound of Formula 1 at about 8 to about 16 hours before such paclitaxel administration; (b) about 100 mg to about 750 mg of the compound of Formula 1 at about 1 to about 3 hours before or with such paclitaxel administration; and (c) about 100 mg to about 750 mg of the compound of Formula 1 at about 6 to about 10 hours after such paclitaxel administration.

171). The method of claim 170, in which such treatment session comprises administering: (a) about 300 mg to about 500 mg of the compound of Formula 1 at about 8 to about 16 hours before such paclitaxel administration; (b) about 300 mg to about 500 mg of the compound of Formula 1 at about 1 to about 3 hours before or with such paclitaxel administration; and (c) about 300 mg to about 500 mg of the compound of Formula 1 at about 6 to about 10 hours after such paclitaxel administration.

172). The method of claim 140, in which such treatment session comprises administering: (a) about 100 mg to about 750 mg of the compound of Formula 1 at about 8 to about 16 hours before such docetaxel administration; (b) about 100 mg to about 750 mg of the compound of Formula 1 at about 1 to about 3 hours before or with such docetaxel administration; and (c) about 100 mg to about 750 mg of the compound of Formula 1 at about 6 to about 10 hours after such docetaxel administration.

173). The method of claim 172, in which such treatment session comprises administering: (a) about 300 mg to about 500 mg of the compound of Formula 1 at about 8 to about 16 hours before such docetaxel administration; (b) about 300 mg to about 500 mg of the compound of Formula 1 at about 1 to about 3 hours before or with such docetaxel administration; and (c) about 300 mg to about 500 mg of the compound of Formula 1 at about 6 to about 10 hours after such docetaxel administration.

174). The method of claim 143, in which such treatment session comprises administering: (a) about 100 mg to about 750 mg of the compound of Formula 1 at about 8 to about 16 hours before such saquinavir administration; (b) about 100 mg to about 750 mg of the compound of Formula 1 at about 1 to about 3 hours before or with such saquinavir administration; and (c) about 100 mg to about 750 mg of the compound of Formula 1 at about 6 to about 10 hours after such saquinavir administration.

175). The method of claim 174, in which such treatment session comprises administering: (a) about 300 mg to about 500 mg of the compound of Formula 1 at about 8 to about 16 hours before such saquinavir administration; (b) about 300 mg to about 500 mg of the compound of Formula 1 at about 1 to about 3 hours before or with such saquinavir administration; and (c) about 300 mg to about 500 mg of the compound of Formula 1 at about 6 to about 10 hours after such saquinavir administration.

176). The method of claim 123 in which the mammal is a human.

177). A method of increasing the delivery of therapeutic and/or preventative treatment across the blood-brain barrier in a mammal, comprising administration to the mammal of an effective amount of a compound of Formula 1 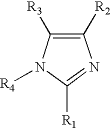
wherein the substituents R₁, R₂, R₃, and R₄ are defined as described in A and B below:
A. when R₁ is selected from the group consisting of:
(i) substituted C₁₋₁₁alkyl or substituted C₂₋₁₁alkenyl, wherein the substituents are selected from the group consisting of hydroxy, C₁₋₆alkyloxy; or
(ii) mono-, di-,and tri-substituted aryl-C₀₋₁₁alkyl wherein aryl is selected from the group consisting of phenyl, furyl, thienyl wherein the substituents are selected from the group consisting of:
(a) phenyl, trans-2-phenylethenyl, 2-phenylethynyl, 2-phenylethyl, or in which the said phenyl group is mono- or disubstituted with a member selected from the group consisting of hydroxy, halo, C₁₋₄alkyl and C₁₋₄alkyloxy,
(b) substituted C₁₋₄alkyl, substituted C₂₋₆alkyloxy, substituted C₂₋₆alkylthio, substituted C₂₋₆alkoxycarbonyl, wherein the substituents are selected from the group consisting of C₁₋₆alkoxy, C₁₋₆alkylthio, or
(c) C₁₋₁₁CO₂R₅, C₁₋₁₁CONHR₅, trans-CH═CHCO₂R₅, or trans-CH═CHCONHR₅ wherein R₅is C₁₋₁₁alkyl, or phenyl C₁₋₁₁alkyl, C₁₋₆alkoxycarbonylmethyleneoxy;
then R₂ and R₃ are each independently selected from the group consisting of mono-, di, and tri-substituted phenyl wherein the substituents are independently selected from:
(i) substituted C₁₋₆alkyl,
(ii) substituted C₁₋₆alkyloxy, C₃₋₆alkenyloxy, substituted C₃₋₆alkenyloxy,
(iii) substituted C₁₋₆alkyl-amino, di(substituted C₁₋₆alkyl)amino,
(iv) C₃₋₆alkenyl-amino, di(C₃₋₆alkenyl)amino, substituted C₃₋₆alkenyl-amino, di(substituted C₃₋₆alkenyl)amino,
(v) pyrrolidino, piperidino, morpholino, imidazolyl, substituted imidazolyl, piperazino, N—C₁₋₆alkylpiperazino, N—C₃₋₆alkenylpiperazino, N—(C₁₋₆alkoxy C₁₋₆alkyl)piperazino, N—(C₁₋₆alkoxy C₃₋₆alkenyl)piperazino, N—(C₁₋₆alkylamino C₁₋₆alkyl)piperazino, N—(C₁₋₆alkylamino C₃₋₆alkenyl)piperazino,
wherein the substituents are selected from the group consisting of
(a) hydroxy, C₁₋₆alkylalkoxy, C₁₋₆alkylamino,
(b) C₃₋₆alkenyloxy, C₃₋₆alkenylamino, or
(c) pyrrolidino, piperidino, morpholino, imidazolyl, substituted imidazolyl, piperazino, N—C₁₋₆alkylpiperazino, N—C₃₋₆alkenylpiperazino, N—(C₁₋₆alkoxy C₁₋₆alkyl)piperazino, N—(C₁₋₆alkoxy C₃₋₆alkenyl)piperazino, N—(C₁₋₆alkylamino C₁₋₆alkyl)piperazino, N—(C₁₋₆alkylamino C₃₋₆alkenyl)piperazino,
or R₂ and R₃ taken together forming an aryl group or substituted aryl, wherein the substituents are defined as above in (i)-(v);
and R₄ is selected from the group consisting of:
(i) hydrogen;
(ii) substituted C₁₋₁₁alkyl or C₂₋₁₁alkenyl wherein the substituents are independently selected from the group consisting of hydrogen, hydroxy, C₁₋₆alkyloxy, C₁₋₆alkylthio, C₁₋₆alkylamino, phenyl-C₁₋₆alkylamino, C₁₋₆alkoxycarbonyl; or
(iii) substituted aryl C₀₋₁₁alkyl wherein the aryl group is selected from phenyl, imidazolyl, furyl, thienyl in which the substituents are selected from A.(a-c); or
B. when R₁ is selected from the group consisting of:
Mono-,di-, and tri-substituted aryl-C₀₋₆alkyl wherein aryl is selected from the group consisting of phenyl, thienyl, and the substituents are selected from the group consisting of:
(a) trans-2-substituted benzimidazolylethenyl, trans-2-substituted benzoxazolylethenyl, trans-2-substituted benzthiazolylethenyl, in which the substituents are selected from the group consisting of hydrogen, hydroxy, halo, trihalomethyl, C₁₋₄alkyl and C₁₋₄alkyloxy, C₁₋₄alkyloxycarbonyl, C₁₋₄alkylamino, di(C₁₋₄alkyl)amino, C₃₋₆alkenylamino, di(C₃₋₆alkenyl)amino, C₁₋₄alkyloxy-C₁₋₄alkylamino, substituted C₁₋₄alkyl and C₁₋₄alkyloxy, substituted C₁₋₄alkyloxycarbonyl, substituted C₁₋₄alkylamino, di(substituted C₁₋₄alkyl)amino, substituted C₃₋₆alkenylamino, di(substituted C₃₋₆alkenyl)amino, wherein the substituents are as defined above,
(b) trans-2-cyano ethenyl, trans-2-alkylsulfonyl ethenyl, trans-2-alkenylsulfonyl ethenyl, trans-2-substituted alkylsulfonyl ethenyl, trans-2-substituted alkenylsulfonyl ethenyl, in which the substituents are defined above,
(c) C₁₋₆CO₂R₅, trans-CH═CHCO₂R₅, C₁₋₆CONHR₅, or trans-CH═CHCONHR₅, wherein R₅ is C₁₋₆alkoxy C₂₋₆alkyl, amino C₂₋₆alkyl, C₁₋₆alkylamino C₂₋₆alkyl, di(C₁₋₆alkyl)amino C₂₋₆alkyl, C₁₋₆alkylthio C₂₋₆alkyl, substituted C₁₋₆alkoxy C₂₋₆alkyl, substituted C₁₋₆alkylamino C₂₋₆alkyl, di(substituted C₁₋₆alkyl)amino C₂₋₆alkyl, substituted C₁₋₆alkylthio C₂₋₆alkyl, in which the substituents are selected from the group consisting of pyrrolidino, piperidino, morpholino, piperazino, N—C₁₋₆alkylpiperazino, N—C₃₋₆alkenylpiperazino, N—(C₁₋₆alkoxy C₁₋₆alkyl)piperazino, N—(C₁₋₆alkoxy C₃₋₆alkenyl)piperazino, N—(C₁₋₆alkylamino C₁₋₆alkyl)piperazino, N—(C₁₋₆alkylamino C₃₋₆alkenyl)piperazino, imidazolyl, oxazolyl, thiazolyl,
(d) C₁₋₆CONR₆R₇, or trans-CH═CHCONR₆R₇, wherein R₆ and R₇ are independently selected from the group consisting of C₁₋₆alkyl, phenyl C₁₋₆alkyl, C₁₋₆alkoxycarbonylmethyleneoxy, hydroxy C₂₋₆alkyl, C₁₋₆alkyloxy C₂₋₆alkyl, amino C₂₋₆alkyl, C₁₋₆alkylamino C₂₋₆-alkyl, di(C₁₋₆alkyl)amino C₂₋₆alkyl, C₁₋₆alkylthio C₂₋₆alkyl, substituted C₁₋₆alkoxy C₂₋₆alkyl, substituted C₁₋₆alkylamino C₂₋₆alkyl, di(substituted C₁₋₆alkyl)amino C₂₋₆alkyl, substituted C₁₋₆alkylthio C₂₋₆alkyl, wherein the substituents are selected from the group consisting of pyrrolidino, piperidino, morpholino, piperazino, N—C₁₋₆alkylpiperazino, N—C₃₋₆alkenylpiperazino, N—(C₁₋₆alkoxy C₁₋₆alkyl)piperazino, N—(C₁₋₆alkoxy C₃₋₆alkenyl)piperazino, N—(C₁₋₆alkylamino C₁₋₆alkyl)piperazino, N—(C₁₋₆alkylamino C₃₋₆alkenyl)piperazino, imidazolyl, oxazolyl, thiazolyl,
(e) R₇C(O)C₁₋₆alkyl, R₇C(O) carbonyl C₂₋₆alkenyl, in which R₇ is defined as above [2(d)],
(f) HO—C₁₋₆alkyl-C₂₋₆alkenyl, R₇—O—C₁₋₆alkyl-C₂₋₆alkenyl, R₇NH—C₁₋₆alkyl-C₂₋₆alkenyl, R₆R₇N—C₁₋₆alkyl-C₂₋₆alkenyl, R₇NH—C(O)—O—C₁₋₆alkyl-C₂₋₆alkenyl, R₆R₇N—C(O)—O—C₁₋₆alkyl-C₂₋₆alkenyl, R₇O—C(O)—O—C₁₋₆alkyl-C₂₋₆alkenyl, R₇—C(O)—O—C₁₋₆alkyl-C₂₋₆alkenyl, wherein R₆ and R₇ is defined as above [2(d)],
(g) R₇O—C₀₋₃alkyl-C₃₋₆cycloalkan-1-yl, R₇NH—C₀₋₃alkyl-C₃₋₆cycloalkan-1-yl, R₆R₇N—C₀₋₃alkyl-C₃₋₆cycloalkan-1-yl, R₇NH—C(O)—O—C₀₋₃C₃₋₆cycloalkan-1-yl, R₆R₇N—C(O)—O—C₀₋₃alkyl-C₃₋₆cycloalkan-1-yl, R₇O—C(O)—O—C₀₋₃alkyl-C₃₋₆cycloalkan-1-yl, R₇—C(O)—O—C₀₋₃alkyl-C₃₋₆cycloalkan-1-yl, R₇O—C(O)—C₀₋₃alkyl-C₃₋₆cycloalkan-1-yl, wherein R₇ and is defined as above [2(d)];
then R₂ and R₃ are each independently selected from the group consisting of:
(1) hydrogen, halo, trihalomethyl, C₁₋₆alkyl, substituted C₁₋₆alkyl, C₁₋₆alkenyl, substituted C₁₋₆alkenyl, C₁₋₆alkyloxy, substituted C₁₋₆alkyloxy, C₃₋₆alkenyloxy, substituted C₃₋₆alkenyloxy, C₁₋₆alkylamino, substituted C₁₋₆alkylamino, C₃₋₆alkenylamino, substituted C₃₋₆alkenylamino,
(2) mono-, di-, and tri-substituted phenyl wherein the substituents are independently selected from:
(i) halo, trifluoromethyl, substituted C₁₋₆alkyl,
(ii) C₁₋₆alkyloxy, substituted C₁₋₆alkyloxy, C₃₋₆alkenyloxy, substituted C₃₋₆alkenyloxy,
(iii) C₁₋₆alkyl-amino, di(C₁₋₆alkyl)amino, substituted C₁₋₆alkyl-amino, di(substituted C₁₋₆alkyl)amino, C₃₋₆alkenyl-amino, di(C₃₋₆alkenyl)amino, substituted C₃₋₆alkenyl-amino, di(substituted C₃₋₆alkenyl)amino, or
(iv) pyrrolidino, piperidino, morpholino, imidazolyl, substituted imidazolyl, piperazino, N—C₁₋₆alkylpiperazino, N—C₃₋₆alkenylpiperazino, N—(C₁₋₆alkoxy C₁₋₆alkyl)piperazino, N—(C₁₋₆alkoxy C₃₋₆alkenyl)piperazino, N—(C₁₋₆alkylamino C₁₋₆alkyl)piperazino, N—(C₁₋₆alkylamino C₃₋₆alkenyl)piperazino,
wherein the substituents are selected from the group consisting of:
(a) hydrogen, hydroxy, halo, trifluoromethyl,
(b) C₁₋₆alkylalkoxy, C₁₋₆alkylamino, C₁₋₆alkylthio,
(c) C₃₋₆alkenyloxy, C₃₋₆alkenylamino, C₃₋₆alkenylthio, or
(d) pyrrolidino, piperidino, morpholino, imidazolyl, substituted imidazolyl, piperazino, N—C₁₋₆alkylpiperazino, N—C₃₋₆alkenylpiperazino, N—(C₁₋₆alkoxy C₁₋₆alkyl)piperazino, N—(C₁₋₆alkoxy C₃₋₆alkenyl)piperazino, N—(C₁₋₆alkylamino C₁₋₆alkyl)piperazino, N—(C₁₋₆alkylamino C₃₋₆alkenyl)piperazino;
with the proviso that at least one of R₂ and R₃ group be selected from
[B (2)] and the phenyl and the substituents be selected from (ii)-(v) above; or R₂ and R₃ taken together forming an aryl group or substituted aryl, wherein the substituents are defined as above in (i)-(iv);
and R₄ is selected from the group consisting of:
(a) hydrogen;
(b) substituted C₁₋₁₁alkyl or C₂₋₁₁alkenyl wherein the substituents are independently selected from the group consisting of hydrogen, hydroxy, C₁₋₆alkyloxy, C₁₋₆alkylthio, C₁₋₆alkylamino, phenyl-C₁₋₆alkylamino, C₁₋₆alkoxycarbonyl and the substituents are selected from (ii)-(iv); or
(c) aryl C₀₋₁₁ alkyl wherein the aryl group is selected from phenyl, imidazolyl, furyl, thienyl
by steps comprising:
(a) choosing a pharmaceutically-active agent that is in the form of a free compound or its pharmaceutically-acceptable pro-drug, metabolite, analogue, derivative, solvate or salt, and that is selected from the group consisting of agents that (i) bind to or are substrates for P-gp, and/or (ii) are taxane analogues; and
(b) administering to such mammal (i) an effective amount of the compound of Formula 1 in the form of a free compound or its pharmaceutically-acceptable pro-drug, metabolite, analogue, derivative, solvate or salt; and (ii) an effective amount of such pharmaceutically-active agent.

178). The method of claim 177, in which such pharmaceutically-active agent is parenterally administered.

179). The method of claim 177, in which such pharmaceutically-active agent is orally administered.

180). The method of claim 177, in which the compound of Formula 1 is administered prior to administration of the pharmaceutically-active agent.

181). The method of claim 177, in which the pharmaceutically-active agent is administered prior to administration of the compound of Formula 1.

182). The method of claim 177, in which the compound of Formula 1 and the pharmaceutically-active agent substantially are simultaneously administered.

183). The method of claim 177, in which the compound of Formula 1 and the pharmaceutically-active agent are administered together in a combined dosage form.

184). The method of claim 177, in which the compound of Formula 1 and the pharmaceutically-active agent are independently administered in separate dosage forms.

185). The method of claim 177, comprising the following additional steps that precede steps (b) and (c):
(i) choosing a regimen of dosage frequency and amount of the pharmaceutically-active agent for such mammal that is therapeutically effective in the absence of the compound of Formula 1, taking into account the systemic toxicity of such pharmaceutically-active agent; and
ii) substantially increasing such dosage frequency or amount of the pharmaceutically-active agent to a toxicity-protected dosage, taking into account the protection against such systemic toxicity provided by such compound of Formula 1.

186). The method of claim 185, in which such dosage frequency of the pharmaceutically-active agent is substantially increased for a given indication.

187). The method of claim 185, in which such dosage amount of the pharmaceutically-active agent is substantially increased for a given indication.

188). The method of claim 177 in which the disease is characterized by the intrinsic presence of multi-drug resistance.

189). The method of claim 177 in which the disease is characterized by the potential to acquire multi-drug resistance.

190). The method of claim 177, in which such pharmaceutically-active agent comprises at least one agent in the form of a free compound or its pharmaceutically-acceptable pro-drug, metabolite, analogue, derivative, solvate or salt selected from the group consisting of: taxanes, epothilones, discodermolide, eleutherobin, sarcodictyins, laulimalides, vinca alkaloids, anthracyclines, camptothecins, epipodophyllotoxins, methotrexate, angiotensin converting enzyme (ACE) inhibitors, human immunodeficiency virus protease inhibitors, antibiotics, calcium channel antagonists, β-blockers, HMG-CoA reductase inhibitors, immunosuppressive agents, opiates, fluoroquinolones, macrolide antibiotics, aminoglycoside antibiotics, antihistamines, anti-epileptic agents, anti-malarial agents, and dopamine agonists.

191). The method of claim 177, in which such pharmaceutically-active agent comprises at least one agent in the form of a free compound or its pharmaceutically-acceptable pro-drug, metabolite, analogue, derivative, solvate or salt selected from the group consisting of: Abeta1-40 (β-amyloid); Abeta1-42 (β-amyloid); Acebutolol; Dactinomycin; Adefovir; Adrenaline; Epinephrine; Albuterol; Salbutamol; Aldosterone; Amikacin; Amitriptyline; Amprenavir; Astemizole; Atorvastatin; Aureobasidin A; Azasetron; Azathioprine; Azidopine; Azithromycin; Bilirubin; Bisantrene; Bunitrolol; Burroughs Wellcome (“BW”) 1019W91; BW 1288U89; BW 1351W91; BW 1379W91; Calcein-AM; Carbamazepine; Carvedilol; Celiprolol; Cerivastatin; Chloroquine; Chlorpromazine; Cimetidine; Clarithromycin; Colchicine; Corticosterone; Cyclosporine; Cyclosporine metabolite AM1; Cytosine arabinoside (cytarabine); Daunorubicin; Debrisoquine; 13-OH-4′-Deoxy-4′-iododoxorubicin; Dexamethasone; Digitoxin; Digoxin; αMethyl-Digoxin; β-acetyl Digoxin; Dihydroindolizino[7,6,5-kl]acridinium chloride; Diltiazem; desacetyl Diltiazem; Dipyridamole; Docetaxel; Domperidone; Doxorubicin; DPDE [D-penicillamine(2,5)]-enkephalin]; D-Penicillamine; Ebastine; Eletriptan; Emetine; Epirubicin; Erythromycin; Estradiol-17-β-D-glucuronide; Etoposide; Felodipine; Fentanyl; Fexofenadine; Flavopiridol; Fluconazole; Fluvastatin; Furosemide; Gemtuzumab ozogamicin; Glibenclamide; Glyburide; Gramicidin D; Grepafloxacin; Hoechst 33342; Hydrocortisone (cortisol); Bayer BAY59-8862 (Indena IDN-5109 paclitaxel analog); Imatinib (Gleevec); Interleukin-2; Interleukin-4; Indinavir; Interferon 2B; Interferon-γ-1B; Irinotecan (CPT-11); Isoniazid; Ivermectin; Labetalol; Dilevalol; L-Dopa (levodopa); Levofloxacin; Loperamide; Loratadine; Losartan; Lovastatin; Mefloquine; Melphalan; Methadone; Methamphetamine; Methotrexate; Methylprednisolone; Mibefradil; Miltefosine; Mitomycin C; Mitoxantrone; Monensin; Morphine; Morphine-6-glucuronide; Moxidectin; MPP+ (1-Methyl-4-phenylpyridium); Nadolol; Naringin; Nelfinavir; Neostigmine; Nicardipine; Nonylphenol ethoxylate; Nortriptyline; Octreotide; Omeprazole; Ondansetron; Paclitaxel; Phenytoin; 2-amino-1-methyl-6-phenylimidazo[4,5-b]pyridine (PhlP); Phosphatidylcholine; Phosphatidylethanolamine; Pirarubicin; Platelet Activating Factor; Plicamycin (Mithramycin); Prazosin; Pristinamycins; Propantheline; Propranolol; PSC833; Puromycin; Quinidine; Quinine; Ranitidine; Reserpine; Retinoic acid; Ritonavir; Saquinavir; Simvastatin; Sirolimus; Somatropin; Sparfloxacin; Tacrolimus; Talinol; Tc-Sestamibi; Terfenadine; Tetracycline; Thapsigargin; Timolol; Tobramycin; Topotecan; Trimethoprim; UK-224,671; Vecuronium; Verapamil; Verapamil metabolite (D-617); Verapamil metabolite (D-620); Vinblastine; Vincristine; Vindesine; and Vinorelbine.

192). The method of claim 190, in which such pharmaceutically-active agent comprises a taxane in the form of a free compound or its pharmaceutically-acceptable pro-drug, metabolite, analogue, derivative, solvate or salt.

193). The method of claim 192, in which such pharmaceutically-active agent comprises paclitaxel in the form of a free compound or its pharmaceutically-acceptable pro-drug, metabolite, analogue, derivative, solvate or salt.

194). The method of claim 193, in which the dosage administered of such paclitaxel during a treatment session is about 120 mg to about 1200 mg per treatment session.

195). The method of claim 194, in which such dosage is about 550 mg to about 1200 mg per treatment session.

196). The method of claim 192, in which such pharmaceutically-active agent comprises docetaxel in the form of a free compound or its pharmaceutically-acceptable pro-drug, metabolite, analogue, derivative, solvate or salt.

197). The method of claim 196, in which the dosage administered of such docetaxel during a treatment session is about 120 mg to about 1200 mg per treatment session.

198). The method of claim 197, in which such dosage is about 550 mg to about 1200 mg per treatment session.

199). The method of claim 191, in which such pharmaceutically-active agent comprises saquinavir in the form of a free compound or its pharmaceutically-acceptable pro-drug, metabolite, analogue, derivative, solvate or salt.

200). The method of claim 199, in which the dosage administered of such saquinavir during a treatment session is about 600 mg to about 2400 mg per treatment session.

201). The method of claim 200, in which such dosage is about 1200 mg to about 2400 mg per treatment session.

202). The method of claim 177 in which the disease is chronic and the pharmaceutically-active agent is administered to the mammal on a long-term basis.

203). The method of claim 177 in which the compound of Formula 1 is selected from the group consisting of: (2-[4-(3-ethoxy-1-propenyl)phenyl]-4,5-bis(4-(2-propylamino)phenyl)-1H-imidazole; 2-[4-(3-ethoxy-trans-1-propen-1-yl)phenyl]-4,5-bis (4-N,N-diethylaminophenyl) imidazole; 2-[4-(3-ethoxy-trans-1-propen-1-yl)phenyl]-4-(4-N,N-diethylaminophenyl)-5-(4-N-methylaminophenyl) imidazole; 2-[4-(3-methoxy-trans-1-propen-1-yl)phenyl]-4,5-bis (4-pyrrolidinophenyl) imidazole; 2-[4-(3-ethoxy-trans-1-propen-1-yl)phenyl]-4,5-bis (4-pyrrolidinophenyl) imidazole; 2-[4-(3-ethoxy-trans-1-propen-1-yl)phenyl]-4-(4-N-dimethylaminophenyl)-5-(4-pyrrolidinophenyl) imidazole; 2-[4-(3-ethoxy-trans-1-propen-1-yl)phenyl]-4-(4-N-methylaminophenyl)-5-(4-pyrrolidino-phenyl) imidazole; 2-[4-(3-ethoxy-trans-1-propen-1-yl)phenyl]-4,5-bis (4-N-morpholinophenyl) imidazole; 2-[4-(3-ethoxy-trans-1-propen-1-yl)phenyl]-4-(4-N-dimethylaminophenyl)-5-(4-N-morpholinophenyl) imidazole; 2-[4-(3-ethoxy-trans-1-propen-1-yl)phenyl]-4-(4-N-methylaminophenyl)-5-(4-N-morpholinophenyl) imidazole; and 2-[4-(3-ethoxy-trans-1-propen-1-yl)phenyl]-4-(4-N-methylaminophenyl)-5-(4-N-isopropylaminophenyl) imidazole.

204). The method of claim 203 in which the compound of Formula 1 has the following formula 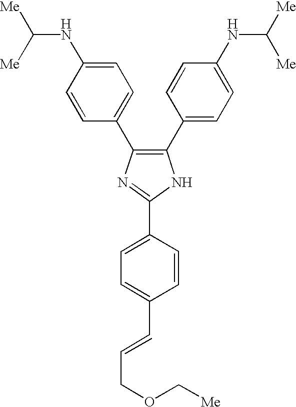
in the form of a free compound or as its pharmaceutically-acceptable pro-drug, metabolite, analogue, derivative or salt.

205). The method of claim 204 in which the compound of Formula 1 is in the form of a mesylate salt.

206). The method of claim 177 in which the disease is a disorder of the central nervous system.

207). The method of claim 206 in which the disease is epilepsy.

208). The method of claim 206 in which the disease is a cognitive disorder.

209). The method of claim 206 in which the disease is Alzheimer's disease.

210). The method of claim 206 in which the disease is Parkinson's disease.

211). The method of claim 177 in which the disease is a viral, bacterial, fungal, or parasitic infection.

212). The method of claim 211 in which the disease is human immunodeficiency virus.

213). The method of claim 177 in which the disease is a cell proliferative disorder and the pharmaceutically-active compound is an anti-cell-proliferative therapeutic agent.

214). The method of claim 213 in which the cell proliferative disorder is a neoplasm.

215). The method of claim 213 in which the cell proliferative disorder is a cancer.

216). The method of claim 213 in which the cell proliferative disorder is metastatic breast cancer.

217). The method of claim 213 in which the cell proliferative disorder is a tumor.

218). The method of claim 213 in which the cell proliferative disorder is a fibrotic disorder.

219). The method of claim 213 in which the cell proliferative disorder is acute myeloid leukemia.

220). The method of claim 213 in which such cells either do not express P-gp, do not express P-gp in all cells, or do not express P-gp at levels sufficient to manifest complete multi-drug resistance.

221). The method of claim 213 in which such cells have not previously been exposed to an anti-cell-proliferative therapeutic agent.

222). The method of claim 213 in which such cells express P-gp and manifest multi-drug resistance.

223). The method of claim 213, in which the anti-cell-proliferative therapeutic agent comprises at least one agent in the form of a free compound or its pharmaceutically-acceptable pro-drug, metabolite, analogue, derivative, solvate or salt selected from the group consisting of: taxanes, epothilones, discodermolide, eleutherobin, sarcodictyins, laulimalides, vinca alkaloids, anthracyclines, camptothecins, and epipodophyllotoxins.

224). The method of claim 213, in which the anti-cell-proliferative therapeutic agent comprises at least one agent in the form of a free compound or its pharmaceutically-acceptable pro-drug, metabolite, analogue, derivative, solvate or salt selected from the group consisting of: paclitaxel, docetaxel, vinblastine, vincristine, vinorelbine, doxorubicin, daunorubicin, etoposide, topotecan, dactinomycin, plicamycin (mithramycin), mitomycin, verapamil, cytosine arabinoside (cytarabine), methotrexate, and irinotecan (CPT-11).

225). The method of claim 193, in which such treatment session comprises administering: (a) about 100 mg to about 750 mg of the compound of Formula 1 at about 8 to about 16 hours before such paclitaxel administration; (b) about 100 mg to about 750 mg of the compound of Formula 1 at about 1 to about 3 hours before or with such paclitaxel administration; and (c) about 100 mg to about 750 mg of the compound of Formula 1 at about 6 to about 10 hours after such paclitaxel administration.

226). The method of claim 225, in which such treatment session comprises administering: (a) about 300 mg to about 500 mg of the compound of Formula 1 at about 8 to about 16 hours before such paclitaxel administration; (b) about 300 mg to about 500 mg of the compound of Formula 1 at about 1 to about 3 hours before or with such paclitaxel administration; and (c) about 300 mg to about 500 mg of the compound of Formula 1 at about 6 to about 10 hours after such paclitaxel administration.

227). The method of claim 196, in which such treatment session comprises administering: (a) about 100 mg to about 750 mg of the compound of Formula 1 at about 8 to about 16 hours before such docetaxel administration; (b) about 100 mg to about 750 mg of the compound of Formula 1 at about 1 to about 3 hours before or with such docetaxel administration; and (c) about 100 mg to about 750 mg of the compound of Formula 1 at about 6 to about 10 hours after such docetaxel administration.

228). The method of claim 227, in which such treatment session comprises administering: (a) about 300 mg to about 500 mg of the compound of Formula 1 at about 8 to about 16 hours before such docetaxel administration; (b) about 300 mg to about 500 mg of the compound of Formula 1 at about 1 to about 3 hours before or with such docetaxel administration; and (c) about 300 mg to about 500 mg of the compound of Formula 1 at about 6 to about 10 hours after such docetaxel administration.

229). The method of claim 199, in which such treatment session comprises administering: (a) about 100 mg to about 750 mg of the compound of Formula 1 at about 8 to about 16 hours before such saquinavir administration; (b) about 100 mg to about 750 mg of the compound of Formula 1 at about 1 to about 3 hours before or with such saquinavir administration; and (c) about 100 mg to about 750 mg of the compound of Formula 1 at about 6 to about 10 hours after such saquinavir administration.

230). The method of claim 229, in which such treatment session comprises administering: (a) about 300 mg to about 500 mg of the compound of Formula 1 at about 8 to about 16 hours before such saquinavir administration; (b) about 300 mg to about 500 mg of the compound of Formula 1 at about 1 to about 3 hours before or with such saquinavir administration; and (c) about 300 mg to about 500 mg of the compound of Formula 1 at about 6 to about 10 hours after such saquinavir administration.

231). The method of claim 177 in which the mammal is a human.
